(19) United States
(12) Patent Application Publication
(10) Pub. No.: US 2026/0337209 A1
(43) Pub. Date: Oct. 29, 2026

(54) BORON-CAPPED CLATHROCHELATE AND MACROCYCLIC LIGANDS AND METAL COMPLEXES THEREOF

(71) Applicants: UVic Industry Partnerships Inc., Victoria (CA); U-REIF INVESTMENTS LIMITED PARTNERSHIP, Vancouver (CA)

(72) Inventor: David Jay Berg, Victoria (CA)

(21) Appl. No.: 18/848,858

(22) PCT Filed: Mar. 21, 2023
(86) PCT No.: PCT/US2023/064777

§ 371 Date: Sep. 19, 2024

Related U.S. Application Data
(60) Provisional application No. 63/322,573, filed on Mar. 22, 2022.

(51) Int. Cl. C07F 5/02; A61K 47/68; A61K 51/10; C07F 1/00; C07F 1/08; C07F 3/00; C07F 3/02; C07F 3/06; C07F 5/00; C07F 5/06; C07F 7/00; C07F 7/24; C07F 15/04; C07F 15/06

(52) U.S. Cl. CPC C07F 5/022; A61K 47/6803; A61K 51/1096; C07F 1/005; C07F 1/08; C07F 3/003; C07F 3/02; C07F 3/06; C07F 5/003; C07F 5/069; C07F 7/003; C07F 7/24; C07F 15/045; C07F 15/065

(57) ABSTRACT

The present technology relates to boron-capped clathrochelate ligands and macrocyclic ligands, and metal complexes thereof, and also to the preparation of such ligands and complexes. In some instances, metal complexes of the ligands can be used as delivery mechanisms for metal ions in medicine, for example, in diagnostic imaging or in therapeutic use.

### FIELD OF THE TECHNOLOGY

The present invention relates to boron-capped clathrochelate and macrocyclic ligands and metal complexes thereof, synthesis of the ligands and complexes and their uses. The metal complexes can be used as delivery mechanisms for metal ions in medicine, for example, in diagnostic imaging and/or in therapeutic use.

### BACKGROUND

Clathrochelate ligands are ligands that can bind or encapsulate metal ions. Existing clathrochelating structures are based primarily on carbon skeletons that include pendant amine or carboxylic acid groups as the binding agents. Such ligands are generally quite challenging to prepare and are typically not amenable to functionalization.

Some examples of known borate cage ligands are borate-capped tris(oxaminates) as shown below:

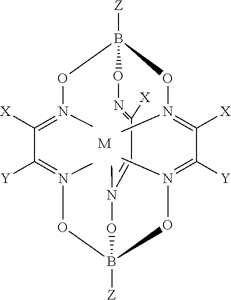

where X=alkyl, aryl, halide; Y=alkyl, aryl, halide; Z=F, alkyl, aryl, heterocycle; M=Fe²⁺, Co²⁺, Cu²⁺.

A range of variants based on this general clathrochelate ligand design are known—they contain ZB—O₃ capping groups with various different oxaminate arms. Because of the size of the cage, these ligands are limited in the type and number of the metal ions they can bind and, because in their synthesis the ligand is formed around the metal, they tend to bind the metal ion irreversibly.

An example of a clathrochelate ligand containing a dipyrazolyl-pyridine arm is the neutral amine-capped ligand shown below (where Bn is a benzyl group, CH₂Ph). Complexes of this ligand with Ln³⁺ ions are known.

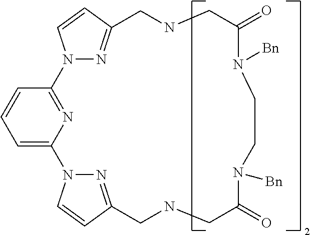

A significant commercial application for existing metal clathrochelate complexes is in MRI contrast agents. Fluorescence sensing agents, based on amine and carboxylate clathrochelate complexes, have been developed. Targeted radiotherapeutic/diagnostic agents using macrocyclic metal complexes have made rapid progress in the treatment of hematological tumors, although treatment of solid tumors is less well established.

### SUMMARY OF THE TECHNOLOGY

In some aspects, the present technology relates to multidentate ligands, in particular clathrochelate ligands and macrocyclic ligands, and to metal salts and metal complexes thereof, and to isomers and isotopes thereof. The clathrochelate ligands have a boron-capped cage structure, in which the boron in each of two caps is bonded to three nitrogen-heterocyclic rings via a B—N bond. The macrocyclic ligands have a boron-capped ring structure, in which the boron in each of two caps is bonded to two nitrogen-heterocyclic rings via a B—N bond.

In some embodiments, a multidentate ligand comprises a first boron atom and a second boron atom, the first and second boron atoms linked by at least two arms, each of the arms comprising a first nitrogen-heterocyclic ring linked by a linking moiety to a second nitrogen-heterocyclic ring, wherein the first nitrogen heterocyclic ring of each arm is bonded to the first boron atom via a B—N bond, and the second nitrogen heterocyclic ring of each arm is bonded to the second boron atom via a B—N bond.

In some embodiments, the multidentate ligand is a clathrochelate ligand in which the first and second boron atoms are linked by three arms, each of the arms comprising a first nitrogen-heterocyclic ring linked by a linking moiety to a second nitrogen-heterocyclic ring, wherein the first nitrogen heterocyclic ring of each of the three arms is bonded to the first boron atom via a B—N bond, and the second nitrogen heterocyclic ring of each of the three arms is bonded to the second boron atom via a B—N bond.

In some embodiments, the multidentate ligand is a macrocyclic ligand in which the first and second boron atoms are linked by two arms, each of the arms comprising a first nitrogen-heterocyclic ring linked a linking moiety to a second nitrogen-heterocyclic ring, wherein the first nitrogen heterocyclic ring of each of the two arms is bonded to the first boron atom via a B—N bond, and the second nitrogen heterocyclic ring of each of the two arms is bonded to the second boron atom via a B—N bond.

In some embodiments of the above-described ligands, the linking moiety is an aromatic or heteroaromatic link. In some embodiments the aromatic or heteroaromatic link is substantially flat or planar. In some embodiments, the link is a pyridine link. In some embodiments, the link is a substituted pyridine link. In some embodiments, the link is a bipyridine link. In some embodiments, the link is a substituted bipyridine link. In some embodiments, the link is a benzene link. In some embodiments, the link is a substituted benzene link.

Various other suitable linking moieties may be used that can accommodate the angular geometry within the arms of the ligand. The type of linking moiety can affect the size of the cage or ring and the metals that can be bound within the ligand.

In some embodiments of the above-described ligands, the linking moiety is aliphatic.

In some embodiments of the above-described ligands, the linking moiety connects the first nitrogen-heterocyclic ring to the second nitrogen-heterocyclic ring in the arms of the ligand via a single atom within the linking moiety. In some embodiments the single atom is a tetravalent atom, such as carbon, silicon or germanium. In some embodiments the atom is a carbon atom.

In some embodiments of the above-described ligands, each of the arms is the same. In some embodiments, one of the at least two arms is different from another of the at least two arms.

In some embodiments of the above-described ligands, the first and second nitrogen-heterocyclic rings are pyrazolyl rings. In some embodiments of the above-described ligands, the first and second nitrogen-heterocyclic rings are triazolyl rings. In some embodiments, a clathrochelate ligand is of Formula I.

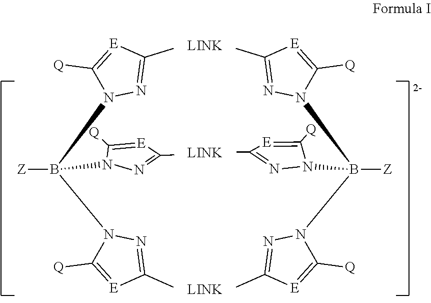

wherein:
E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
Q=H, alkyl, aryl, alkenyl, or alkynyl;
Z=H, alkyl, aryl, alkoxy, N-heterocycle or

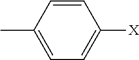

where X=H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK=

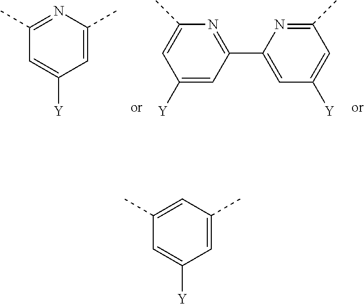

or LR′₂ where Y=H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6, O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C, Si or Ge and each R′ is individually selected from H, alkyl or alkoxy.

In some embodiments, a clathrochelate ligand is of Formula I wherein LINK=LR′₂.

In some embodiments, a clathrochelate ligand is of Formula I wherein L=C.

In some embodiments, a clathrochelate ligand is of Formula I wherein Z═H, and L=C.

In some embodiments, a clathrochelate ligand is of Formula I wherein Z═H, E=C-J, and L=C.

In some embodiments, a clathrochelate ligand is of Formula I wherein Z═H, E=CH, Q=H, and L=C

In some embodiments, a clathrochelate ligand is of Formula I wherein: LINK=

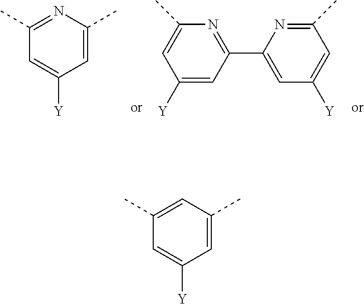

where Y=H, alkyl, aryl, alkenyl, alkynyl, CF₃, F, Cl, Br, I, O(CH₂)ₙR (n=0-4), S(CH₂)ₙR (n=0-4), NH(CH₂)ₙNR₂ (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6, O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

In some embodiments, a clathrochelate ligand is of Formula I wherein Z═H.

In some embodiments, a clathrochelate ligand is of Formula I wherein Z═H and E=C-J.

In some embodiments, a clathrochelate ligand is of Formula I wherein Z═H, E=CH, Q=H, and
LINK=

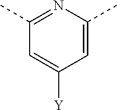

where Y=H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring (see Formula II below).

In some embodiments, a clathrochelate ligand is of Formula I wherein Z═H, E=CH, Q=H, and
LINK=

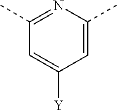

wherein Y═H, alkyl, aryl, alkenyl, CF₃, F, Cl, Br, I, O(CH₂)ₙR (n=0-4), S(CH₂)ₙR (n=0-4), NR(CH₂)ₙNR₂, NH(CH₂)ₙOH (n=0-4), NO₂, SO₃R, CN, C(O)R, C(O)NR₂, C(═NHNH₂)NH₂ (n=0-4), CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, or aryl.

In some embodiments, a clathrochelate ligand is of Formula I wherein Z═H, E=CH, Q=H, and
LINK=

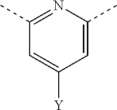

wherein Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl.

In some embodiments, a clathrochelate ligand is of Formula I wherein Z═H, E=CH, Q=H, and
LINK=

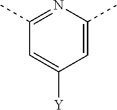

wherein Y═H

In some embodiments, a clathrochelate ligand is of Formula I wherein Z═H, E=C-J, and Q and/or J≠H, and LINK=2,6 pyridinyl (see Formula III below).

In some embodiments, a clathrochelate ligand is of Formula I wherein Z═H, E=C-J, J=H, alkyl, alkenyl, F, Cl, Br, I, SH, NH₂, or C(O)CF₃, Q=H, alkyl, or alkenyl, LINK=2,6 pyridinyl, and wherein Q and/or J≠H.

In some embodiments, a clathrochelate ligand is of Formula I wherein Z═H, E=C-J, and J=H or Br and Q=Me; or J=Br and Q=H; or J=C(═O)CF₃ and Q=H.

In some embodiments, a clathrochelate ligand is of Formula I wherein Z═H, E=CH, Q=H, and
LINK=

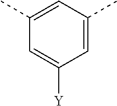

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring (see Formula IV below).

In some embodiments, a clathrochelate ligand is of Formula I wherein Z═H, E=CH, Q=H, and
LINK=

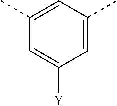

wherein Y═H, alkyl, alkenyl, CF₃, Br, I, NO₂, CN, C(O)Me, C(O)NH₂, (CH₂)OR, OR, (CH₂)SR, SR, NR₂, where each R is individually selected from H, alkyl, alkenyl, or aryl.

In some embodiments, a clathrochelate ligand is of Formula I wherein Z═H, E=N, Q=H, and
LINK=

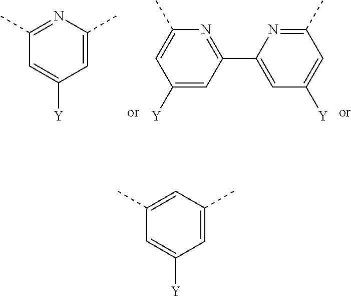

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring (see Formula V below).

In some embodiments, a clathrochelate ligand is of Formula I wherein Z═H, E=N, Q=H, and
LINK=

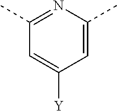

where Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl,

In some embodiments a macrocyclic ligand is of Formula VI:

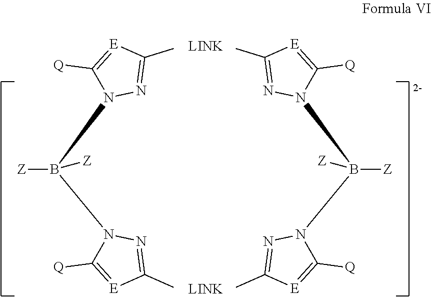

wherein:
   E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
   Q=H, alkyl, aryl, alkenyl, or alkynyl;
   Z═H, alkyl, aryl, alkoxy, N-heterocycle or

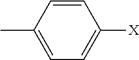

where X=H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK=

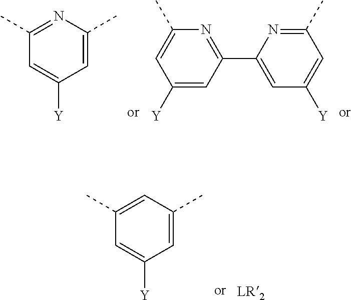

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C, Si or Ge and each R′ is individually selected from H, alkyl or alkoxy In some embodiments, a macrocyclic ligand is of Formula VI wherein LINK=LR′₂.

In some embodiments, a macrocyclic ligand is of Formula VI wherein L=C.

In some embodiments, a macrocyclic ligand is of Formula VI wherein Z═H, and L=C.

In some embodiments, a macrocyclic e ligand is of Formula VI wherein Z═H, E=C-J, and L=C.

In some embodiments, a macrocyclic ligand is of Formula VI wherein Z═H, E=CH, Q=H, and L=C

In some embodiments, a macrocyclic ligand is of Formula VI where at least one of Z is H, and wherein:
LINK=

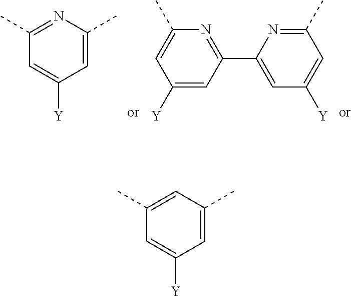

where Y═H, alkyl, aryl, alkenyl, alkynyl, CF₃, F, Cl, Br, I, O(CH₂)ₙR (n=0-4), S(CH₂)ₙR (n=0-4), NH(CH₂)ₙNR₂ (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6, O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

In some embodiments, a macrocyclic ligand is of Formula VI wherein Z═H.

In some embodiments, a macrocyclic ligand is of Formula VI wherein Z═H and E=C-J.

In some embodiments, a macrocyclic ligand is of Formula VI wherein Z═H, E=CH, Q=H, and
LINK=

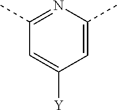

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring (see Formula VII below).

In some embodiments, a macrocyclic ligand is of Formula VI wherein Z═H, E=CH, Q=H, and
LINK=

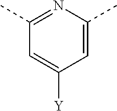

wherein Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl.

In some embodiments, a macrocyclic ligand is of Formula VI wherein Y═H, Cl, Br, I or CN.

In some embodiments, a metal salt of a ligand is a metal salt of any of the clathrochelate ligands and macrocyclic ligands described above.

In some embodiments, a metal complex of a ligand is a metal complex of any of the clathrochelate ligands and macrocyclic ligands described above.

In some embodiments, the clathrochelate ligands and macrocyclic ligands described above are functionalized to include a connector for binding the ligand to a biomolecule. In some embodiments, metal complexes of the clathrochelate ligands and macrocyclic ligands described above are functionalized to include a connector for binding the metal complex of the ligand to a biomolecule.

In some embodiments, a radiopharmaceutical composition comprises a solution of a metal complex of any of the clathrochelate ligands and macrocyclic ligands described above, wherein the metal is a radioactive metal. In some such embodiments, the solution is an aqueous solution.

In some embodiments, a radiopharmaceutical composition comprises a solution of a metal complex of any of the clathrochelate ligands and macrocyclic ligands described above, wherein the ligand comprises at least one radioactive atom. In some such embodiments, the solution is an aqueous solution.

In some embodiments, a radiopharmaceutical composition comprises a solution of a metal complex of any of the clathrochelate ligands and macrocyclic ligands described above bound to a biomolecule, wherein the metal is a radioactive metal. In some such embodiments, the solution is an aqueous solution. The biomolecule, in some embodiments, is a monoclonal antibody and the ligand is bound to the biomolecule via a connector.

In some embodiments, a radiopharmaceutical composition comprises a solution of a metal complex of any of the clathrochelate ligands and macrocyclic ligands described above bound to a biomolecule, wherein the ligand comprises at least one radioactive atom. In some such embodiments, the solution is an aqueous solution. The biomolecule, in some embodiments, is a monoclonal antibody and the ligand is bound to the biomolecule via a connector.

In some embodiments, a kit for preparing an antibody-drug conjugate comprises a radionuclide complex of any of the clathrochelate ligands and macrocyclic ligands described above, the ligand optionally modified to include a connector for binding the ligand to a biomolecule, the kit comprising:
(a) a first solution of a metal salt of the ligand, the ligand optionally modified to include a connector for binding the ligand to a biomolecule, wherein the metal is a displaceable metal; and
(b) a second a solution of a salt of a radionuclide;
wherein upon mixing the first and second, the displaceable metal is displaced by the radionuclide to form the radionuclide complex.

In some embodiments, of such a kit, the radionuclide is useful for radiotherapy or diagnostic imaging procedures.

In some embodiments of a method to treat a patient suffering from a disease condition, the method comprises injecting into the patient an effective amount of any of the radiopharmaceutical compositions described above. In some embodiments, the radiopharmaceutical composition is injected in saline.

In some embodiments of a method to diagnose a condition in a patient, the method comprising injecting into the patient an effective amount of any of the radiopharmaceutical compositions described above. In some embodiments, the radiopharmaceutical composition is injected in saline.

Thus, the present technology includes metal salts of clathrochelate ligands and macrocyclic ligands according to any of the formulae and embodiments described herein, and to isomers and isotopes thereof. Some embodiments of the present technology are potassium or lithium salts of clathrochelate ligands and macrocyclic ligands according to any of the formulae and embodiments described herein. In some embodiments the ligand itself comprises one or more radioactive isotopes. For example, the ligand could comprise one or more radioiodine or radiobromine substituents.

Some embodiments of the present technology relate to methods for preparation of a clathrochelate ligand according to the formulae herein, the methods as described herein. Some embodiments of the present technology relate to methods for preparation of a macrocyclic ligand according to the formulae herein, the methods as described herein.

Some embodiments of the present technology relate to methods for preparation of a metal complex comprising a clathrochelate ligand according to the formulae herein and at least one metal, the methods as described herein. Some embodiments of the present technology relate to methods for preparation of a metal complex comprising a macrocyclic ligand according to the formulae herein and at least one metal, the methods as described herein.

The present technology includes metal complexes of clathrochelate ligands and macrocyclic ligands according to any of the formulae and embodiments described herein, and to isomers and isotopes thereof. In some embodiments the metal in the metal complex is a transition metal. In some embodiments the metal in the metal complex is a lanthanide. In some embodiments the metal in the metal complex is an actinide. In some embodiments the metal is a main group metal or semi-metal. In some embodiments metal in the metal complex is a radioactive isotope. In some embodiments the metal is a radionuclide selected from the group consisting of Cu, Tc, Gd, Ga, In, Co, Re, Fe, Au, Ag, Rh, Pt, Bi, Cr, W, Ni, V, Ir, Zn, Cd, Mn, Ru, Pd, Hg, and Ti.

The present technology also includes pharmaceutical compositions including a complex comprising at least one clathrochelate ligand or macrocyclic according to any of the formulae and embodiments described herein and optionally at least one metal, and a pharmaceutically acceptable carrier, diluent or excipient.

The present technology also includes diagnostic and/or therapeutic agents comprising metal complexes of clathrochelate ligands and macrocyclic ligands described herein, bound to a biomolecule via a suitable connector or linker. In some embodiments the biomolecule is a monoclonal antibody. In some embodiments the metal complex is a radioactive metal complex of the ligand.

This summary does not necessarily describe the entire scope of all aspects of the technology. Other aspects, features, and advantages will be apparent to those of ordinary skill in the art upon review of the following description. Further aspects and example embodiments are illustrated in the accompanying drawings and/or described in the following description.

### BRIEF DESCRIPTION OF THE DRAWINGS

The accompanying drawings illustrate non-limiting example embodiments of the technology.

FIG. 1 shows the crystal structure of a dipotassium salt of an embodiment of a boron-capped clathrochelate ligand as described herein.

FIG. 2 shows the crystal structure of a dimeric barium (II) salt of an embodiment of a boron-capped clathrochelate ligand as described herein.

FIG. 3 shows the crystal structure of a barium (II) salt of another embodiment of a boron-capped clathrochelate ligand as described herein.

FIG. 4 shows the crystal structure of a dimeric dipotassium salt of an embodiment of a boron-capped clathrochelate ligand as described herein.

FIG. 5 shows the crystal structure of a cobalt (II) salt of an embodiment of a boron-capped clathrochelate ligand as described herein.

FIG. 6 shows the crystal structure of a lead (II) salt of an embodiment of a boron-capped clathrochelate ligand as described herein.

FIG. 7 shows the crystal structure of a yttrium(III) complex of an embodiment of a boron-capped clathrochelate ligand as described herein.

FIG. 8 shows the crystal structure of a cerium(III) complex of an embodiment of a boron-capped clathrochelate ligand as described herein.

FIG. 9 shows the crystal structure of a silver (I) salt of an embodiment of a boron-capped clathrochelate ligand as described herein.

FIG. 10 shows the crystal structure of a copper (II) salt of an embodiment of a boron-capped clathrochelate ligand as described herein.

FIG. 11 shows the crystal structure of a polymeric dipotassium salt of a boron-capped macrocyclic ligand as described herein.

### DETAILED DESCRIPTION OF ILLUSTRATIVE EMBODIMENT(S)

Throughout the following description, specific details are set forth in order to provide a more thorough understanding of the technology. However, the technology may be practiced without these particulars. In other instances, well known aspects or features have not been shown or described in detail to avoid unnecessarily obscuring the invention. Accordingly, the specification and drawings are to be regarded in an illustrative, rather than a restrictive sense.

### LIST OF ABBREVIATIONS

ADC—antibody-drug conjugate
amu—atomic mass units.
Bp.—boiling point
ca.—circa, meaning about or approximately.
COSY—correlation spectroscopy.
δ—NMR chemical shift in parts per million.
DCM—dichloromethane or methylene chloride
DMAc·DMA—dimethylacetamide dimethyl acetal, Me₂NCMe(OMe)₂.
DMF·DMA—dimethylformamide-dimethyl acetal, Me₂NCH(OMe)₂.
DMSO or dmso—dimethylsulfoxide.
Equiv.—equivalents.
ESI-MS—electrospray ionization mass spectrometry (positive or negative mode).
HMBC—heteronuclear multiple bond correlation.
HSQC—heteronuclear single bond correlation.
LAH—lithium aluminium hydride.
Mp—melting point.
MRI—magnetic resonance imaging.
m/z—mass/charge ratio (in amu).
NMR—nuclear magnetic resonance.
PET—positron-emission tomography.
py—pyridine or pyridyl
pz—pyrazole or pyrazolyl
SPECT—single photon emission computed tomography.
TGA—thermogravimetric analysis.
THF—tetrahydrofuran.

### Definitions

As used herein, “alkyl” means a straight chain or branched alkyl group. In some embodiments alkyl groups have from 1 to 12 carbon atoms, in some embodiments from 1 to 10 carbon atoms, in some embodiments from 1 to 8 carbon atoms, in some embodiments from 1 to 6 carbon atoms, in some embodiments from 1 to 4 carbon atoms, and in some embodiments from 1 to 3 carbon atoms. Examples of straight chain alkyl groups include, but are not limited to, methyl, ethyl, n-propyl, and n-butyl groups. Examples of branched alkyl groups include, but are not limited to, isopropyl, iso-butyl, sec-butyl, t-butyl, neopentyl, isopentyl, and 2,2-dimethylpropyl groups.

As used herein, “alkenyl” means a straight chain or branched alkenyl group. In some embodiments alkenyl groups have from 2 to 10 carbon atoms, in some embodiments from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated groups that contain at least one carbon-carbon double bond. Examples of alkenyl groups include, but are not limited to, vinyl, allyl, propenyl, and butenyl.

As used herein, “alkynyl” means a straight chain or branched alkynyl group. In some embodiments alkynyl groups have from 2 to 10 carbon atoms, in some embodiments from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated groups that contain at least one carbon-carbon triple bond. Examples of alkynyl groups include, but are not limited to, ethynyl, propynyl, and butynyl.

As referred to, alkyl, alkenyl and alkynyl groups can be hydrocarbon groups, but they are not limited to being hydrocarbon groups. For example, they can be partially fluorinated or perfluorinated, and/or can contain other elements or substituents besides hydrogen attached to the carbon chain.

“Halo” or “halogen” refers to fluorine, chlorine, bromine, and iodine.

“Hydroxy” refers to —OH.

“Cyano” refers to —CN.

As used herein, “alkoxy” refers to an alkyl as defined above joined by way of an oxygen atom (i.e., —O-(alkyl). Examples of alkoxy groups include, but are not limited to, methoxy, ethoxy, n-propoxy, n-butoxy, isopropoxy, sec-butoxy, tert-butoxy, and the like.

As used herein, “aryl” groups are cyclic aromatic groups that do not contain heteroatoms in the ring. Thus, aryl groups include, but are not limited to, phenyl, biphenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl. Some or all of the ring hydrogens in an aryl group may be replaced by halogens and/or other elements or substituents besides hydrogen.

As used herein, “heterocycle,” or “heterocyclic” refers to aromatic and non-aromatic ring moieties containing 3 or more ring members, of which one or more is a heteroatom such as, but not limited to, N, O, S, or P. In some embodiments, heterocycle include 3 to 20 ring members, whereas other such groups have 3 to 15 ring members. At least one ring contains a heteroatom, but every ring in a polycyclic system need not contain a heteroatom. Heterocyclic groups also include fused ring species including those having fused aromatic and non-aromatic groups. A heterocyclic group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom.

As used herein, “heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“Isomer” is used herein to refer to molecules with the same molecular formula (i.e. the same number of atoms of each element), but different structural or spatial arrangements of the atoms within the molecule, The term “isomer” encompasses structural isomers and stereoisomers including all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention.

“Isotope” refers to atoms with the same number of protons but a different number of neutrons, and an isotope of a compound of any of the formulae included herein includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes—protium (with one proton and no neutrons), and deuterium (with one proton and one neutron); as well, hydrogen has a radioactive isotope, tritium (with one proton and two neutrons). The term “isotope” encompasses radioactive isotopes, also known as radioisotopes or radionuclides. For example, ¹²⁵I and ¹³¹I are iodine radioisotopes, and ⁶⁴Cu and ⁹⁰Y are metal isotopes.

“Salt” as used herein refers to a chemical compound consisting of an ionic assembly of one or more cations and anions. Salts are composed of related numbers of cations (positively charged ions) and anions (negatively charged ions) so that the product is electrically neutral (without a net charge). In some embodiments a salt comprises an anion, wherein the anion is a clathrochelate or macrocyclic ligand as described herein, in combination with a counter ion such as, but not limited to a potassium or lithium ion.

“Metal complex” as used herein refers to a chemical compound formed by the assembly of one or more neutral metal atoms or charged metal ions with a ligand in which the ligand donates one or more electron pairs to the metal to form an electrostatic bond. The overall complex may be neutral, positively or negatively charged. In embodiments of the technology described herein, a clathrochelate cage serves as a multidentate ligand towards one, or up to three, metal ions to form a metal complex. In other embodiments of the technology described herein, a macrocyclic ligand serves as a multidentate ligand binding at least a single metal ion to form a metal complex.

### Boron-Capped Multidentate Ligands

The technology described herein relates to a family of clathrochelate (cage) ligands and macrocyclic (ring) ligands, and to metal salts and metal complexes thereof, and to isomers and isotopes thereof.

These multidentate ligands comprise two boron atoms. The two boron atoms are linked by three arms in the case of the clathrochelate ligands, or by two arms in the case of the macrocyclic ligands. In some embodiments, the arms in the ligands are the same as one another. In some embodiments, one of the arms is different from the other arm(s), for example, it may include an additional functional group. The arms in the ligands each comprise a pair of nitrogen-heterocyclic rings linked by a linking moiety. In some embodiments, the linking moiety is an aromatic or heteroaromatic link. In some embodiments the aromatic or heteroaromatic link is substantially flat or planar. In some embodiments the link is a substituted pyridine. In some embodiments, the linking moiety links the two nitrogen-heterocyclic rings of the arm via a single carbon atom. The nitrogen-heterocyclic ring at one end of each arm is bonded to the first boron atom, and the nitrogen-heterocyclic ring at the other end of each arm is bonded to the second boron atom. Thus, the clathrochelate ligands have a boron-capped cage structure, in which the boron in each of two caps is bonded to three nitrogen-heterocyclic rings via a B—N bond. The macrocyclic ligands have a boron-capped ring structure, in which the boron in each of two caps is bonded to two nitrogen-heterocyclic rings via a B—N bond.

This family of novel clathrochelate and macrocyclic ligands provides a highly versatile chemical “toolbox” that can be used to build compounds that are suitable for use in various diagnostic and therapeutic medicinal applications, as well as in wide range of other potential end-use applications. The ligands provide several chemical “handles” that can be adjusted in many different combinations, thus providing a plethora of possible compounds. For example, through the choice of ligand framework itself, and/or the choice of metal(s) bound to the ligand, and/or the choice of functional groups and substituents on the ligand, the ligand can be built and tailored to suit a specific end-use application and/or to have particular desired properties. For example, the functional groups on the ligands can be chosen to confer solubility in water or other solvents, stability in aqueous solution and/or in vivo, and in some cases to provide connectors or linkers to other moieties (e.g. monoclonal antibodies).

Functionalization of the ligands can be advantageous or even essential for some of the end-use applications. For example, introduction of synthetic handles or reactive moieties that can be used to connect solubilizing and/or biological targeting groups is likely to be particularly important for medical applications, because some unfunctionalized embodiments of the ligands have limited solubility in water. Similar strategies can be used to introduce other features, such as polymerizable or surface-binding pendant groups, if those features are desirable for certain end-use applications of the ligands and their complexes. The boron-capped clathrochelate and macrocyclic ligands can be functionalized at one or more different sites as described in further detail below. For the clathrochelate ligands, examples of various synthetic approaches to functionalization are described. In one approach, at least one component of the cage already has a functional group or is functionalized before cage assembly, and then the components are assembled to form the cage. In another approach the cage is formed, and then functional groups are introduced into the intact cage. In some cases a functional group can be introduced at a particular site via the first approach, and then a further functional group can be introduced into the intact cage at a different site, via a post-cage formation modification reaction. In some cases a functional group can be introduced at a particular site via the first approach, and then modified once the cage is formed, via a post-cage formation modification reaction. In some cases a functional group can be introduced at a particular site via the second approach, and then further modified by further reaction. Similar approaches can be adopted to functionalize the macrocyclic ligands described herein.

Embodiments of boron-capped clathrochelate ligands and macrocyclic ligands are described in further detail below.

### Boron-Capped Clathrochelate Ligands

Several novel types of boron-capped clathrochelate ligands have been developed, synthesized, and converted to metal complexes. As described above, these clathrochelate ligands comprise a boron-capped cage structure, where the boron in each of two caps is bonded to three nitrogen-heterocyclic rings via a B—N bond. Each nitrogen-heterocyclic ring on the first boron is indirectly connected to a heterocyclic ring on the second boron by a linking moiety, so that the six heterocyclic rings form part of three arms that link the two boron atoms, forming a cage structure. Each of the three arms thus comprises at least two nitrogen-heterocyclic rings. In some embodiments, the linking moiety is another nitrogen-heterocyclic ring, such as a pyridine ring. In other embodiments, the linking moiety is a benzene ring or another (preferably planar) aromatic or heteroaromatic moiety, such as a bipyridine or other suitable linkage. In some embodiments, the linking moiety links the two nitrogen-heterocyclic rings of the arm via a single atom in the linking moiety, such as a carbon atom. The arms of the cage and/or the boron caps can be functionalized to modify the properties of the clathrochelate ligand. The cage structures of these multidentate ligands can provide an interior of suitable size and environment to bind one or more metal ions.

For example, embodiments of the present technology include clathrochelate ligands of Formula I, metal salts and metal complexes of the same, and isomers and isotopes thereof:

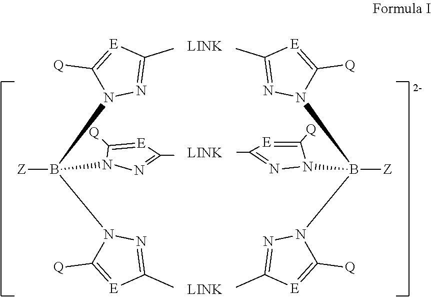

wherein:
E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
Q=H, alkyl, aryl, alkenyl, or alkynyl;
Z═H, alkyl, aryl, alkoxy, N-heterocycle or

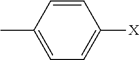

where X═H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK=

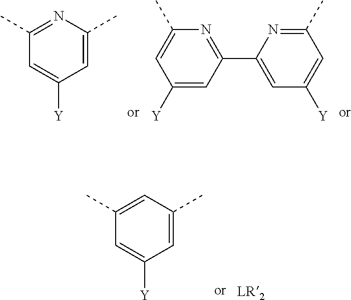

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl or a heterocyclic ring,
and where L=C, Si or Ge and each R′ is individually selected from H, alkyl or alkoxy.
   Clathrochelate Ligands with Pyridine Link and Pyrazole Rings—Optionally Functionalized at Pyridine

Some embodiments of the present technology include clathrochelate ligands of Formula I where there is a hydrogen on each boron (Z═H), a pyridine group in each LINK, and a pair of pyrazolyl rings in each of the three arms of the cage (E=CH and Q=H).

For example, embodiments of the present technology include clathrochelate ligands of Formula II (and metal salts and metal complexes of the same, and isomers and isotopes thereof):

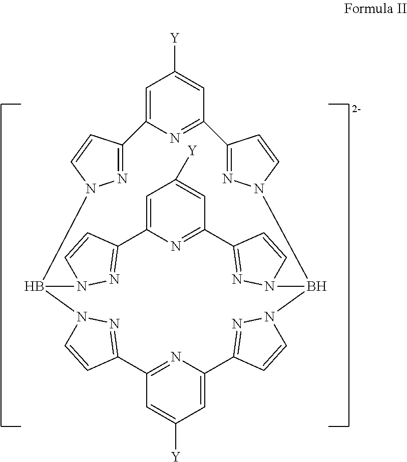

Clathrochelate ligand [4a] is an embodiment of Formula II, wherein Y═H.

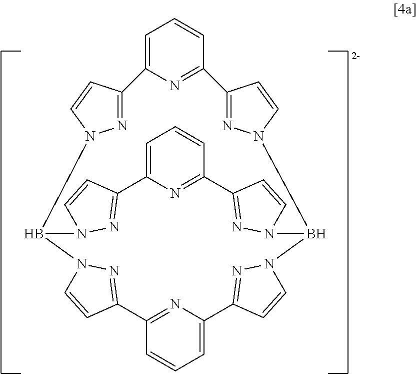

In clathrochelate ligands which have a pyridine link in each of the three arms, the para- or 4-position of each of the three pyridine rings is an attractive site for functionalization of cage ligand, for example, to give clathrochelate ligands of Formula II in which Y≠H.

Several examples of clathrochelate ligands of Formula II have been synthesized, generally as their potassium salt. These ligands [4a, 4aa and 4b—z] are listed in Table 1 below.

TABLE 1
Clathrochelate Ligands of Formula II

| Ligand | Y substituent | Synthetic Route | Example # |
|---|---|---|---|
| 4a | H | Scheme 2 or 3 | 9 & 12 |
| 4aa | t-butyl | Scheme 2 | 10 |
| 4b | Cl | Scheme 3 | 13 |
| 4c | Br | Scheme 3 | 14 |
| 4d | I | Scheme 3 | 15 |
| 4e | CN | Scheme 3 | 16 |
| 4f | CF₃ | Scheme 2 | 11 |
| 4g | C(═O)OLi | Scheme 4B | 17 |
| 4h | C(═O)CH₃ | Scheme 4A | 18 |
| 4i | C(═O)NH₂ | Scheme 4A | 19 |
| 4j | C(═NHNH₂)NH₂ | Scheme 4A | 20 |
| 4k | O(CH₂)₄CH═CH₂ | Scheme 4B | 21 |
| 4l | O(CH₂CH₂O)₂CH₃ | Scheme 4B | 22 |
| 4m | O(CH₂CH₂O)₄CH₃ | Scheme 4B | 23 |
| 4n | O(CH₂CH₂O)₆CH₃ | Scheme 4B | 24 |
| 4o | O(CH₂CH₂O)₂CH═CH₂ | Scheme 4B | 25 |
| 4p | O(CH₂)₃OH | Scheme 4B | 26 |
| 4q | OCH₂-Anthracene | Scheme 4B | 27 |
| 4r | S(CH₂)₃SH | Scheme 4B | 28 |
| 4s | SCH₂Ph | Scheme 4B | 29 |
| 4t | SCH₂-4-MeOC₆H₄ | Scheme 4B | 30 |
| 4u | SCH₂CH₂(1,3-dioxapentane) | Scheme 4B | 31 |
| 4v | 4-pyridone | Scheme 4B | 32 |
| 4w | O(CH₂)₄CH═CHC(═O)OCH₃ | From [4k] | 33a |
| 4x | NH(CH₂)₂NH₂ | From [4c] | 33b |
| 4y | NH(CH₂)₃NH₂ | From [4c] | 33c |
| 4z | NMe(CH₂)₂NHMe | From [4c] | 33d |

The synthesis can begin with formation of a corresponding arm (or a “precursor” arm) as illustrated in Scheme 1.

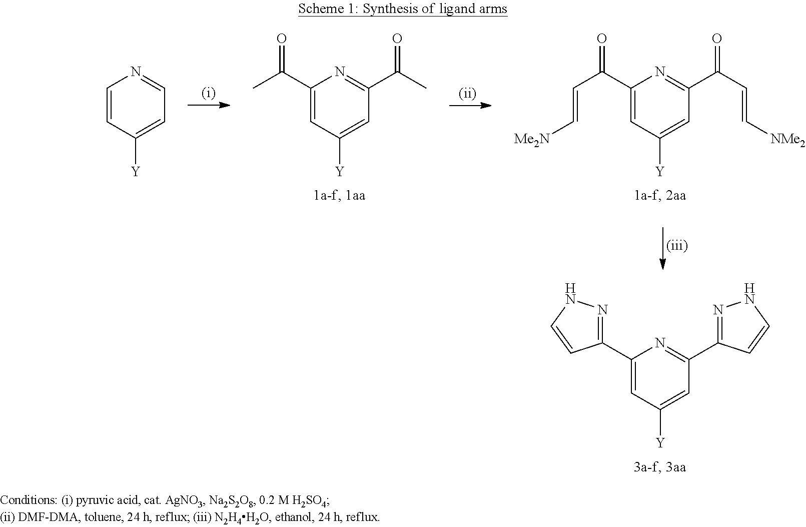

2,6-Diacetylpyridine [1a] is commercially available. However, some 2,6-diacetyl-4-substituted pyridines (e.g. 1aa, 1e and 1f) are either not presently commercially available or are extremely expensive. It is possible to diacetylate 4-substituted pyridines at the 2- and 6-positions using the Minisci reaction (see, for example, D. Gygi, S. J. Hwang and D. G. Nocera, J. Org. Chem. 2017, 82, 12933-12938) but the yields tend to be low (20-40%), particularly if the substituent in the 4-position is electron donating. Once the 2,6-diacetyl-4-substituted pyridine has been prepared, it can be converted to the corresponding arm as shown in Scheme 1.

The clathrochelate ligands in Table 1 have then been prepared using an appropriate arm via several different synthetic routes.

Some of the ligands in Table 1 have been prepared by a melt reaction of potassium borohydride (KBH₄) with the arm in 2:3 ratio, as shown in Scheme 2.

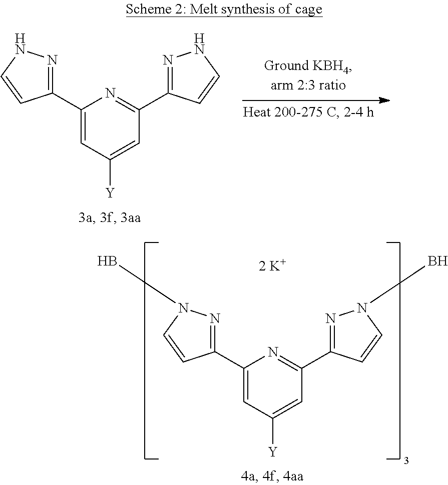

Some of the ligands in Table 1 have been prepared by a solution phase reaction of potassium borohydride (KBH₄) (2 equiv) with the dried arm (3 equiv) in diglyme (Bp. 162° C.) or triglyme (Bp. 216° C.), with 2 mol % Y(OTf)₃ as catalyst, as shown in Scheme 3. This solution reaction is useful for arms that do not melt below 300° C. or that decompose on prolonged heating.

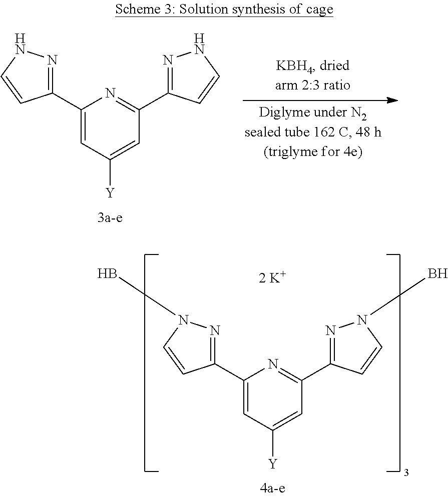

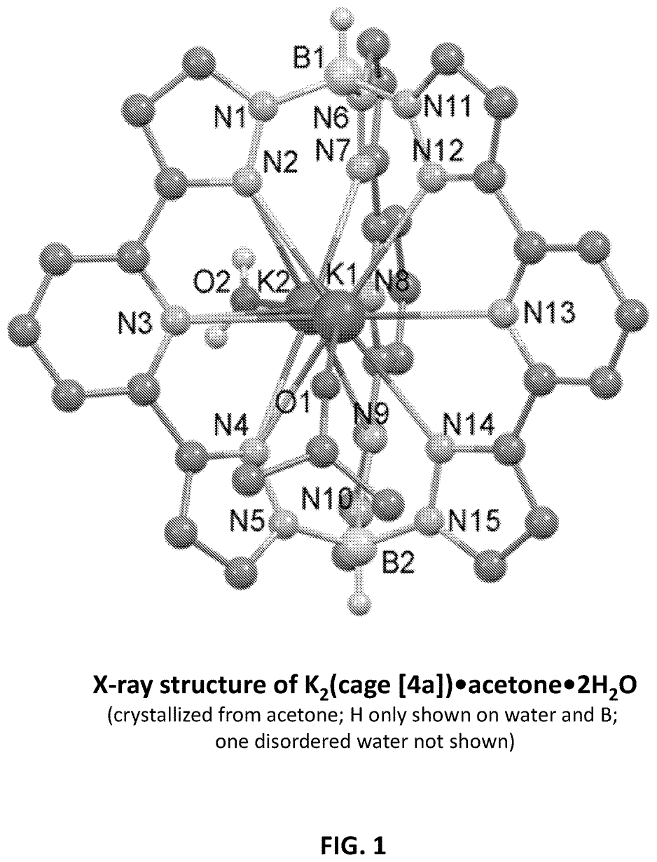

Clathrochelate ligand [4a] can be prepared as a dipotassium salt, K₂ cage [4a] on at least a multi-gram scale by preparing the arm [3a] via the reaction shown in Scheme 1 (Example 1), then using the melt reaction shown in Scheme 2 (see Example 9) or the solution phase reaction shown in Scheme 3 (see Example 12). The synthetic approach in Scheme 2 involves thermal elimination of 6 equivalents of hydrogen gas from the reaction of 2-(pyrazol-5′-yl)-6-(pyrazol-5′-yl)pyridine (2) with potassium borohydride (KBH₄) in a 3:2 molar ratio at ca. 250° C. in the melt at ambient pressure in air. The final product can be purified by extraction into hot isopropanol followed by immediate precipitation on cooling to room temperature, to yield the dipotassium salt of clathrochelate ligand, K₂ cage [4a] as a white powder. An X-ray crystal structure of the dipotassium salt of cage [4a] is shown in FIG. 1. The crystal grew in an acetone solution, and there is an acetone molecule in the crystal structure.

The dipotassium salt of the clathrochelate ligand, K₂ cage [4a], is very soluble in acetone and DMSO, moderately soluble in ethyl acetate, THF and acidic water, slightly soluble in alcohols (MeOH, EtOH, i-PrOH) and halocarbons (CH₂Cl₂ and CHCl₃). It has very limited solubility in pure water.

In cases where the arm melts and the melt reaction can be used, the yields have been found to be generally comparable for the melt and solution reactions. The use of the melt reaction avoids the need to remove the diglyme solvent at high temperature under vacuum.

Some of the ligands in Table 1 have been prepared by functionalization of a precursor clathrochelate ligand as shown in Scheme 4A and Scheme 4B.

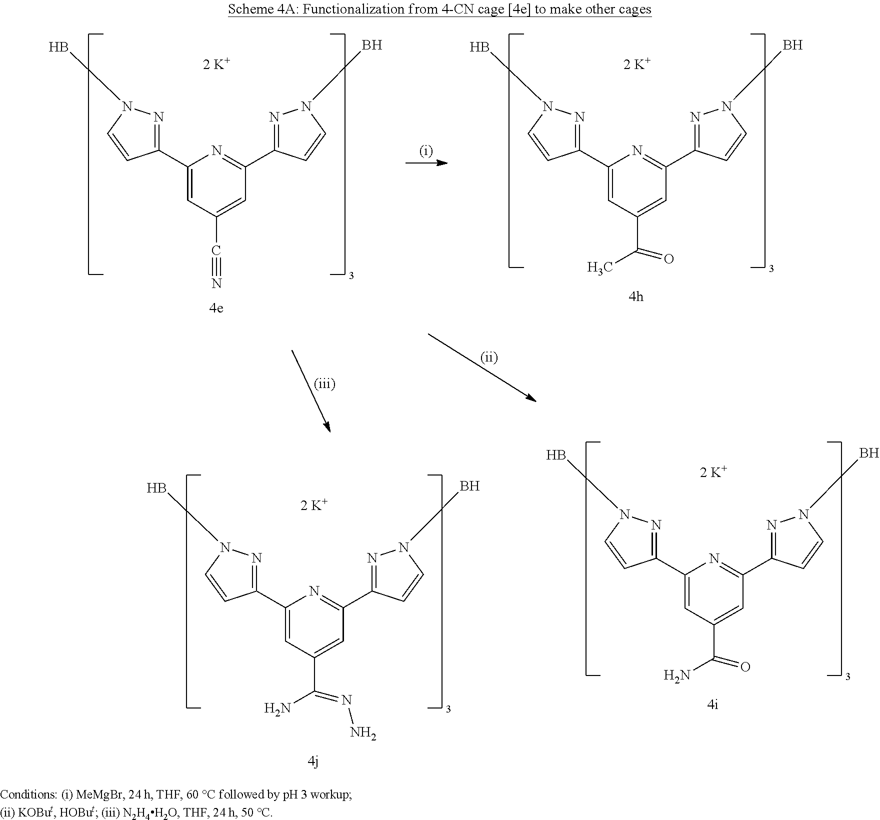

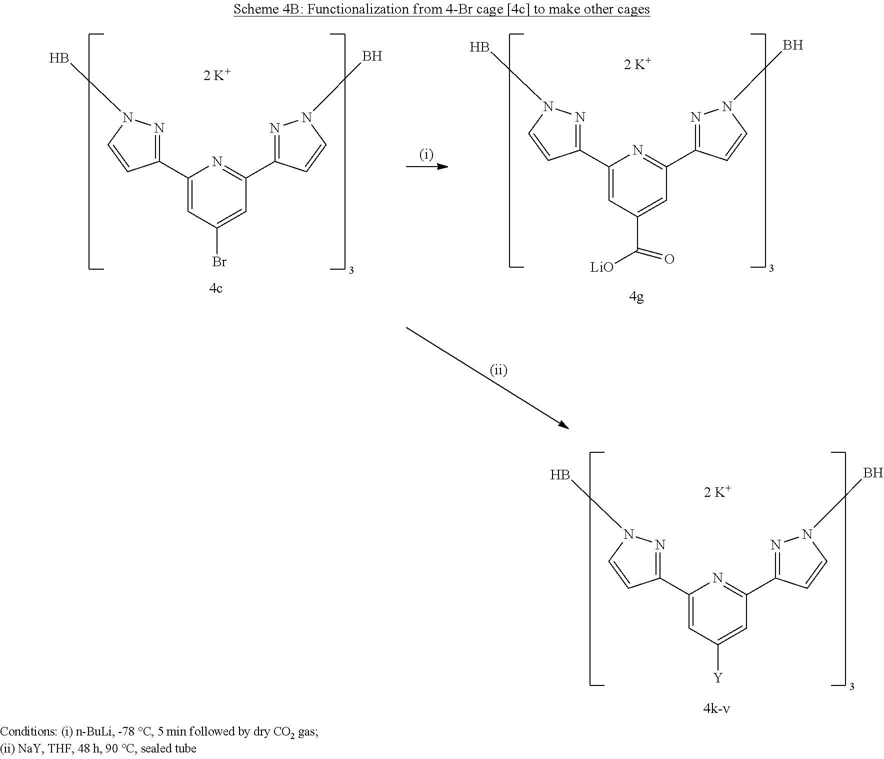

Some of these ligands (e.g. 4k, 4o, 4p, 4r, 4w-z) provide functional handles that can be used for connecting suitable linkers for bioconjugation. Other routes to functionalized ligands are described in reference to Scheme 11 below. For example, ligand [4w] can be prepared from [4k] and the resulting alpha,beta-unsaturated ketone group is expected to react with cysteine-SH groups

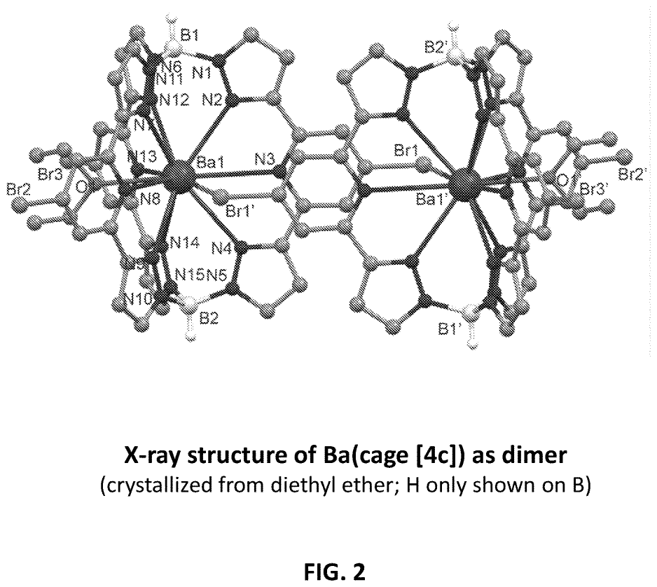

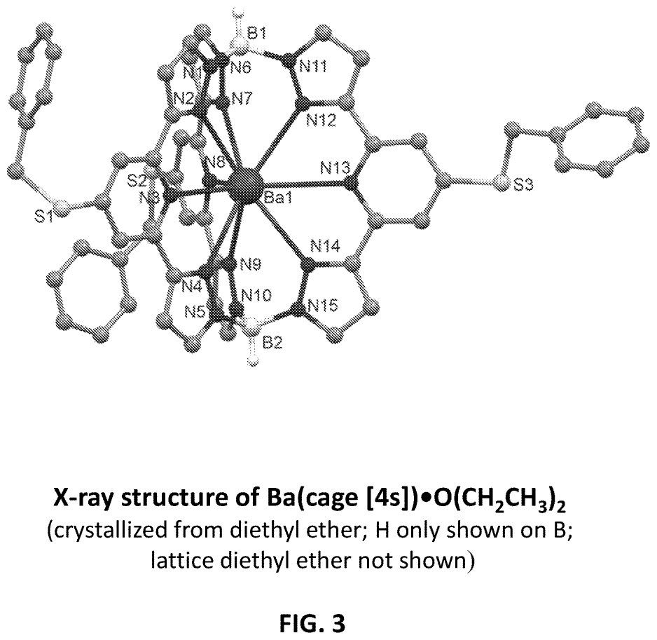

An X-ray crystal structure of a dimeric barium salt of cage [4c] is shown in FIG. 2. An X-ray crystal structure of a barium salt of cage [4s] is shown in FIG. 3.

Clathrochelate Ligands with Pyridine Link and Pyrazole Rings—Functionalized at Pyrazole Ring

Some embodiments of the present technology include clathrochelate ligands of Formula I where there is a hydrogen on each boron (Z═H), a pyridine group in each LINK, and a pair of pyrazolyl rings in each of the three arms of the cage (E=C−J), wherein the pyrazole rings in the arms are substituted at the 4′-position and/or at the 5′-position (so Q and/or J≠H). For example, embodiments of the present technology include clathrochelate ligands of Formula III (and metal salts and metal complexes of the same, and isomers and isotopes thereof):

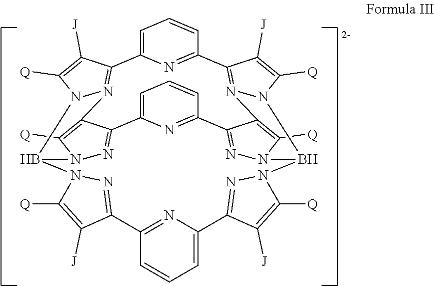

wherein Q and/or J≠H.

Several examples of clathrochelate ligands of Formula III have been synthesized, generally as their potassium salt. These ligands are listed in Table 2 below.

TABLE 2
Clathrochelate Ligands of Formula III

| | | | Synthetic | |
|---|---|---|---|---|
| Ligand | J substituent | Q substituent | Route | Example # |
| 5a | H | Me | Scheme 5A | 34 |
| 5b | Br | H | Scheme 5B | 35 |
| 5c | C(═O)CF₃ | H | Scheme 5B | 36 |
| 5d | Br | Me | Scheme 5B | 37 |

The 5′-methylpyrazole cage [5a] can be synthesized as shown in Scheme 5A below, by preparing the arm [3′a]—by substituting the DMF·DMA in Scheme 1 with dimethylacetamide dimethyl acetal (DMAc·DMA))—then performing a melt reaction of potassium borohydride (KBH₄) with arm [3′a] in 2:3 ratio.

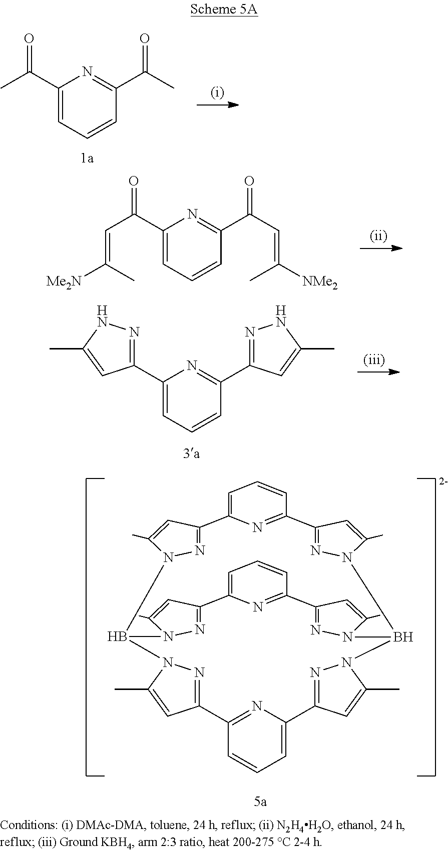

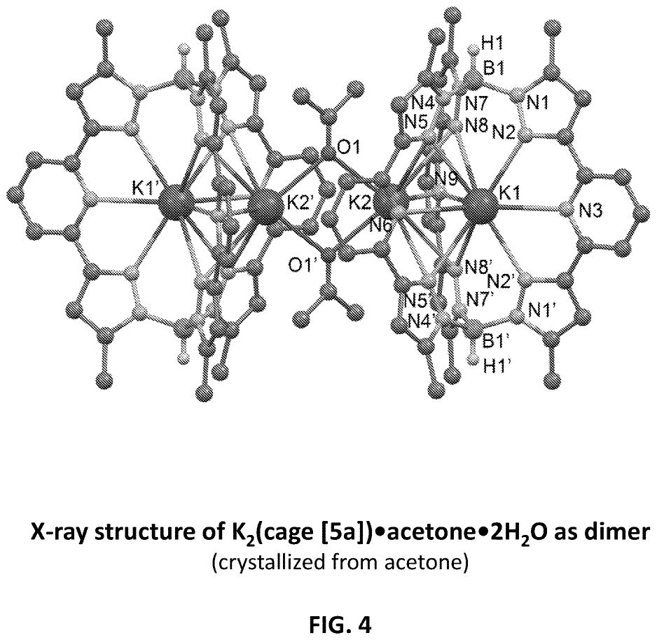

An X-ray crystal structure of the K₂ cage [5a] is shown in FIG. 4. The structure is a dimer comprising two cages, with one potassium in the middle of each cage, and one potassium between the arms that then bridges through two acetones to the other cage.

The melting point of the arms seems to be lower in the 5′-methyl substituted variant K₂ cage [5a], relative to the unsubstituted K₂ cage [4a]. The 5′-methyl substitution also appears to facilitate the reaction with potassium borohydride (KBH₄) in the melt reaction to form the cage, providing easier isolation and purification of cage [5a] as the potassium salt.

In other embodiments of clathrochelate ligands of Formula III where substituents are incorporated on the pyrazole rings in the arms, the rings are substituted at the 4′-position.

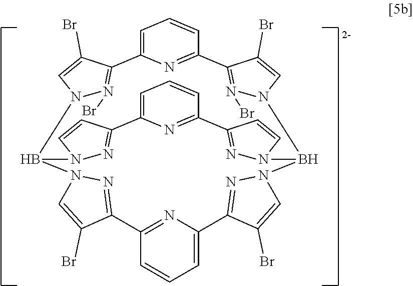

For example, this 4′-bromopyrazole cage [5b] (Formula III wherein Q=H and J=Br) can be synthesized as shown in Scheme 5B below, by treating cage [4a] with 6 equivalents of N-bromosuccinimide (NBS). This results in bromination at the 4′-position of each of the six pyrazolyl rings to give clathrochelate ligand [5b].

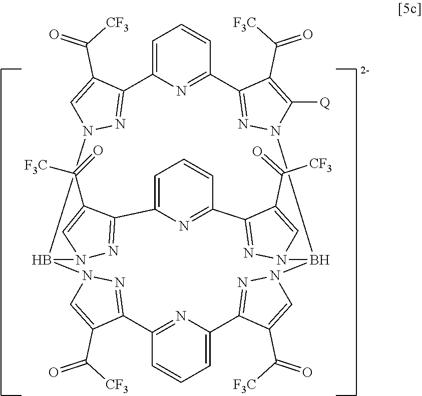

This 4′-trifluoroacetyl pyrazole cage [5c] can be synthesized as shown in Scheme 5B below, by electrophilic acetylation of cage [4a] using excess trifluoroacetic anhydride in dry pyridine. This results in substitution at the 4′-position of the pyrazolyl rings to give predominantly the hexakis trifluoroacetylation product [5c], with some of the pentakis trifluoroacetylation product also in evidence. The trifluoroacetyl groups can be converted to hydrazones or oximes by reaction with hydrazine or hydroxylamine, respectively, and these could serve as connectors (also known as linkers) to biological targeting agents, for example.

In other embodiments of clathrochelate ligands of Formula III where substituents are incorporated on the pyrazole rings in the arms, they are substituted at both the 4′-position and the 5′-position.

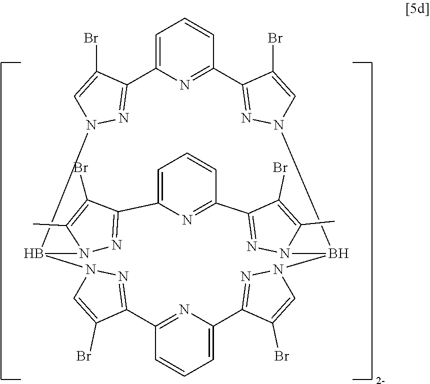

For example, this 4′-bromo-5′-methylpyrazolyl cage [5d] (Formula III wherein Q=Me and J=Br) can be synthesized as shown in Scheme 5B below, by treating the 5′-methyl cage ligand [5a] with 6 equivalents of N-bromosuccinimide (NBS) in acetonitrile. This results in selective bromination of the pyrazolyl rings at the 4′-position (rather than bromination of the methyl groups) to form clathrochelate ligand [5d]. Bromination proceeded rapidly at room temperature with or without visible light illumination.

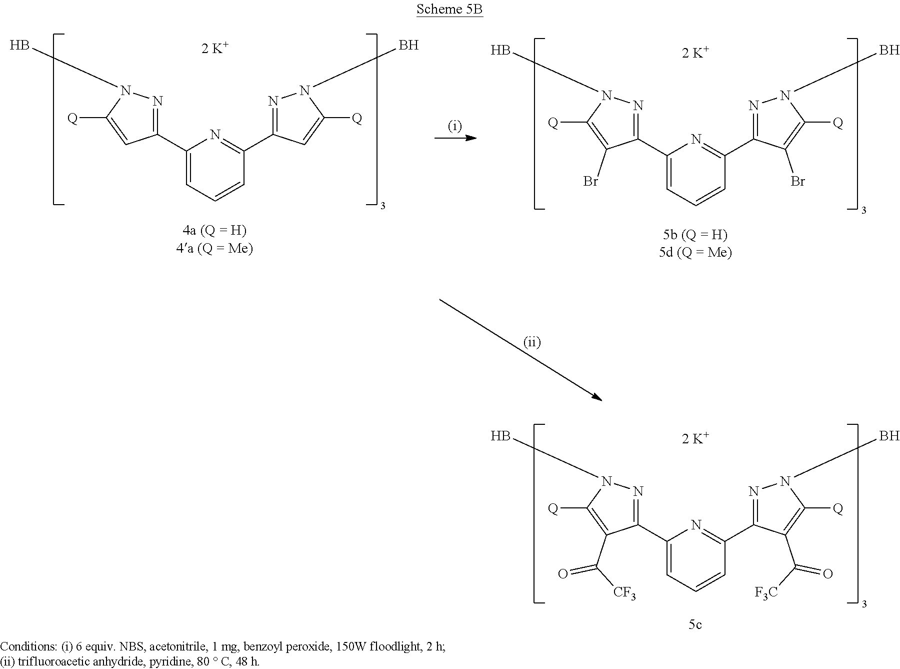

Clathrochelate Ligands with Benzene Link and Pyrazole Rings—Optionally Functionalized at Benzene

Other embodiments of the present technology include clathrochelate ligands of Formula I where there is a hydrogen on each boron (Z═H), a pair of pyrazolyl rings in each of the three arms of the cage (E=CH and Q=H), and the LINK is a 1,3-benzene unit.

For example, embodiments of the present technology include clathrochelate ligands of Formula IV (and metal salts and metal complexes of the same, and isomers and isotopes thereof).

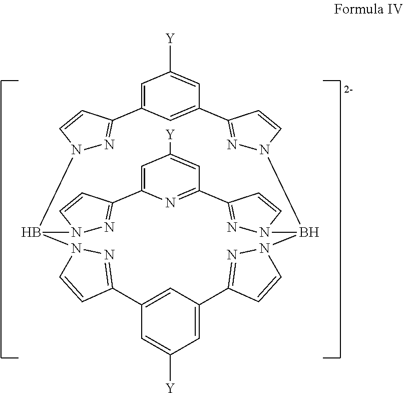

For example, cage ligand [6a], which is an embodiment of Formula IV wherein Y═H, can be made by using a procedure similar to that shown in Scheme 1 to prepare the arm [14] using commercially available 1,3-diacetylbenzene, and then performing a melt reaction of potassium borohydride (KBH₄) with the arm in a 2:3 ratio as shown in Scheme 6, and as described in Example 38 below.

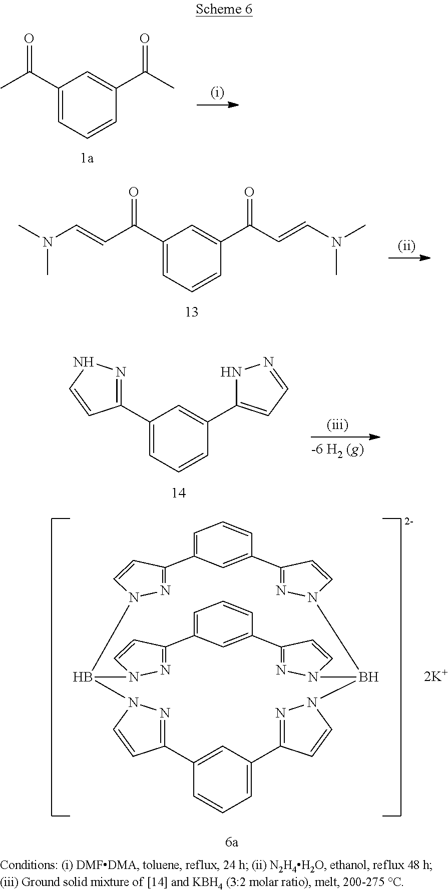

Clathrochelate Ligands with Benzene or Pyridine Link and Triazole Rings

In other embodiments of the clathrochelate ligands of Formula I, the arms comprise 1,2,4-triazole rings (i.e. where Z═H, E=N and Q=H in Formula I). For example, embodiments of the present technology include clathrochelate ligands of Formula Va and Formula Vb below (and metal salts and metal complexes of the same, and isomers and isotopes thereof):

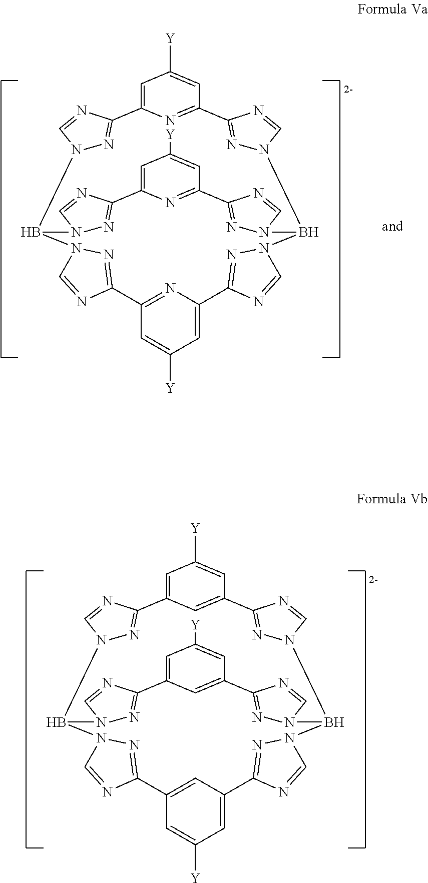

For example, cage ligand [7a], which is an embodiment of Formula Vb wherein Y═H, can be made by using a procedure as shown in Scheme 7, and as described in Example 39a below.

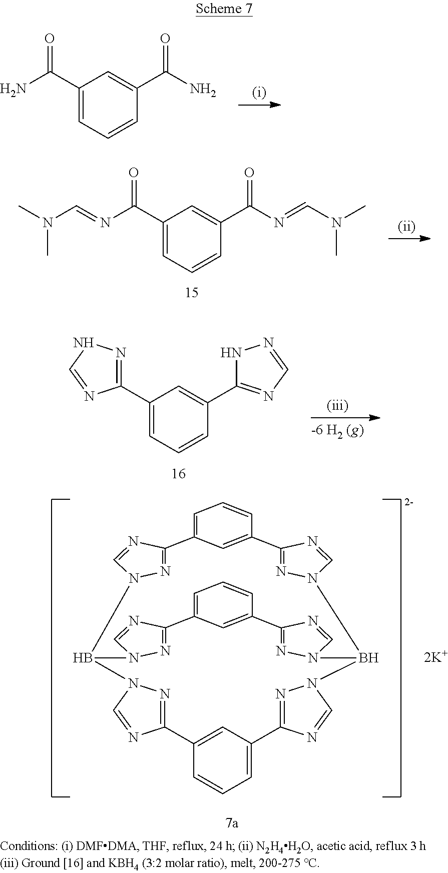

Clathrochelate Ligands with Other Linking Moieties

Some embodiments of the present technology include clathrochelate ligands of Formula I where there is a hydrogen on each boron (Z═H), a pair of pyrazolyl rings (i.e. where E=CH and Q=H) or a pair of 1,2,4-triazole rings (i.e. where E=N and Q=H) in each of the three arms of the cage, and the LINK is LR′₂ where L=C, Si or Ge, and each R′ is individually selected from H, alkyl or alkoxy.

In some embodiments of Formula I there is a hydrogen on each boron (Z═H), a pair of pyrazolyl rings (i.e. where E=CH and Q=H) in each of the three arms of the cage, and the LINK is LR′₂ where L=C and each R′ is individually selected from H, alkyl or alkoxy.

For example, cage ligand [7′a] can be made by using a procedure as described in Example 39b below.

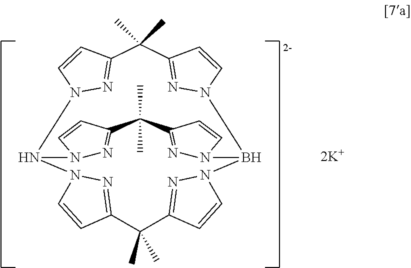

### Macrocyclic Ligands

Several novel boron-capped macrocyclic ligands have been synthesized. As described above, embodiments of these macrocyclic ligands comprise a boron-capped ring structure, where the boron in each of two caps is bonded to two nitrogen-heterocyclic rings via a B—N bond. Each nitrogen-heterocyclic ring on the first boron is indirectly connected to a heterocyclic ring on the second boron by a linking moiety, so that the heterocyclic rings form part of two arms that link the two boron atoms, forming a ring structure. Each of the two arms thus comprises at least two nitrogen-heterocyclic rings. The arms of the ring can be functionalized to modify the properties of the macrocyclic ligand. The ring structures of these multidentate ligands can provide an interior of suitable size and environment to bind a metal ion.

For example, embodiments of the present technology include macrocyclic ligands of Formula VI, metal salts and metal complexes of the same, and isomers and isotopes thereof:

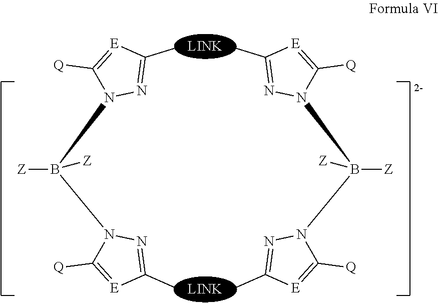

wherein:
E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
   Q=H, alkyl, aryl, alkenyl, or alkynyl;
   Z═H, alkyl, aryl, alkoxy, N-heterocycle or

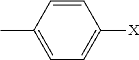

where X═H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK=

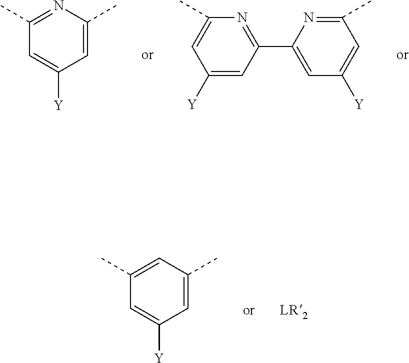

where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C, Si or Ge, and each R′ is individually selected from H, alkyl or alkoxy.

Some embodiments of the present technology include macrocyclic ligands of Formula VI where there are two hydrogens on each boron (Z═H), a pyridine group in each LINK, and a pair of pyrazolyl rings in each of the two arms of the ring (E=CH and Q=H). For example, embodiments of the present technology include macrocyclic ligands of Formula VII (and metal salts and metal complexes of the same, and isomers and isotopes thereof):

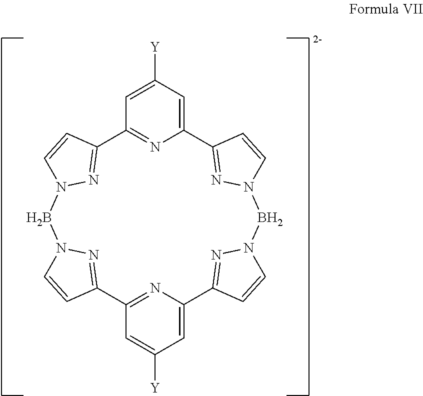

For example, macrocyclic ligand [8a] is an embodiment of Formula VII, wherein Y═H.

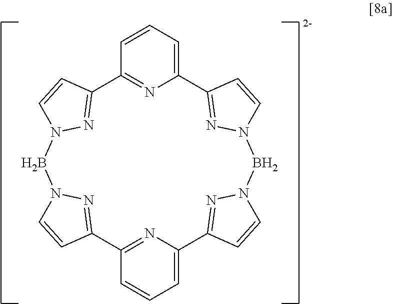

In macrocyclic ligands which have a pyridine link in each of the two arms, the para- or 4-position of each of the two pyridine rings is an attractive site for functionalization of ring, for example, to give macrocyclic ligands of Formula VII in which Y≠H.

Several examples of macrocyclic ligands of Formula VII have been synthesized. These ligands [8a-e] are listed in Table 3 below.

TABLE 3
Clathrochelate Ligands of Formula VII

| Ligand | Y substituent | Synthetic Route | Example # |
|---|---|---|---|
| 8a | H | Scheme 8 | 40 |
| 8b | Cl | Scheme 8 | 41 |
| 8c | Br | Scheme 8 | 42 |
| 8d | I | Scheme 8 | 43 |
| 8e | CN | Scheme 8 | 44 |

The rings were synthesized as their potassium salt in solution, as shown in Scheme 8 (which is similar to Scheme 2), using a 1:1 ratio of potassium borohydride (KBH₄) and the appropriate arm in diglyme (Bp. 162° C.), the corresponding arms [3a-e]being made as shown in Scheme 1 above.

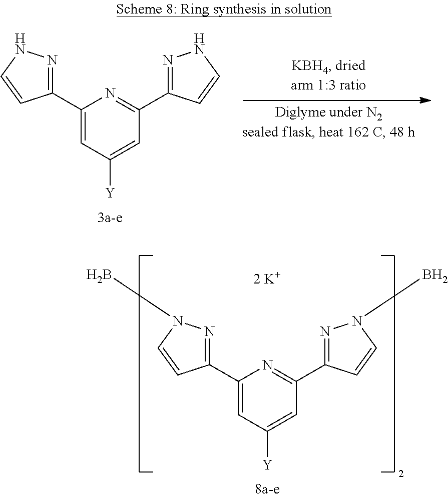

The general procedure for preparing the rings in diglyme solution involved combining the dried arm (2 equiv), potassium borohydride (KBH₄) (2 equiv) and 2 mol % Y(OTf)₃ as catalyst. Diglyme (approx. 10 mL/mmol of arm) was added and the reaction mixture heated at 160° C. for 48 h. In most cases, outgassing was observed and all arm dissolved. On cooling, the solvent was removed. The oily solid residue was dissolved in acetone, insoluble solids were removed by filtration, and the acetone solvent was removed under vacuum. The crude product was extracted into diethyl ether, filtered and the solvent removed under vacuum. The solid product was washed with hexane and dried under vacuum to afford the ring product as an off-white to yellow solid. Specific details of the synthesis and characterization of macrocyclic ligands [8a-e] is provided in Examples 40a and 41-44 below.

Some embodiments of the present technology include macrocyclic ligands of Formula VI where there is a pair of pyrazolyl rings (i.e. where E=CH and Q=H) or a pair of 1,2,4-triazole rings (i.e. where E=N and Q=H) in each of the arms of the ring, and the LINK is LR′₂ where L=C, Si or Ge, and each R′ is individually selected from H, alkyl or alkoxy. In some embodiments there is a hydrogen on each boron (Z═H).

In some embodiments of Formula VI there is a pair of pyrazolyl rings (i.e. where E=CH and Q=H) in each of the two arms of the ring, and the LINK is LR′₂ where L=C and each R′ is individually selected from H, alkyl or alkoxy. In some embodiments there is a hydrogen on each boron (Z═H). In some embodiments there is a N-heterocycle bonded to each boron.

For example, macrocyclic ligand [8′a] can be made by using a procedure as described in Example 40b below.

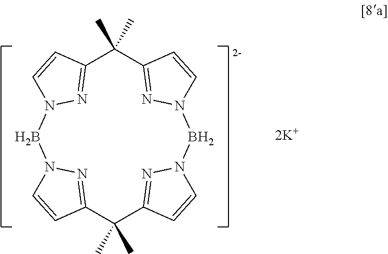

Macrocyclic ligand [8″a], having a (μ-N1,N1-2,2-dipyrazolylpropane bonded to each boron, can be made by using a procedure as described in Example 40c below.

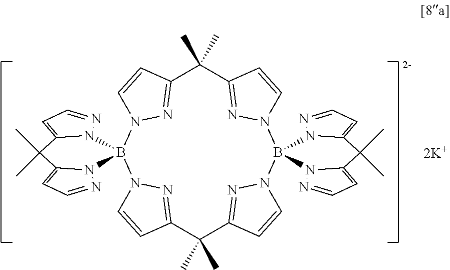

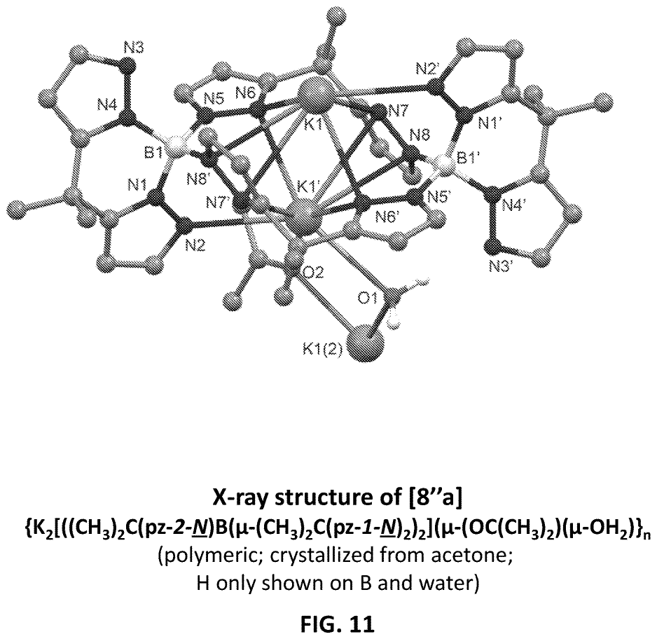

An X-ray crystal structure of [8″a], crystallized from acetone, is shown in FIG. 11

It may be possible to convert embodiments of the macrocyclic ligands described herein into clathrochelate ligands by introducing a third arm connecting the two boron atoms. The third arm may be different from the other two arms, for example, it may include a functional group that is not present in the other two arms. In some cases it may be beneficial to have just a single functional group or reactive site on the cage ligand, rather than three. Under some reaction conditions, instead of introduction of a single new type of arm, redistribution of the arms may occur resulting in a mixture of cage ligands—some having all of one type of arm, some having all of the other type or arm, and some having one arm of one type and two of the other, and vice versa.

### Metal Complexes of Clathrochelate and Macrocyclic Ligands

Metal complexes of the above-described clathrochelate and macrocyclic ligands can be prepared with a wide variety of metals, including transition metals, lanthanides and radioactive metals and metal isotopes. In some embodiments a single metal ion can be bound in a single cage ligand or a single ring ligand, resulting in mononuclear complexes. In some embodiments, multiple metal ions can be bound in a single cage ligand, resulting in homopolynuclear complexes. This provides a unique coordination environment compared to conventional sepulchrates and other clathrochelates that bind a single metal in the center of a donor array. This increase in metal payload per cage ligand, relative to conventionally used clathrochelate ligands, can be beneficial for diagnostic or therapeutic medical applications of the metal complexes. In some embodiments, it may be possible to bind two (or more) different metal ions simultaneously, in a single cage. Such heteropolynuclear clathrochelate complexes, could potentially be used to provide diagnostic and therapeutic functions at the same time (a theranostic approach) in their end-use applications.

Metal complexes of cage [4a] can be prepared by adding an acetone solution of K₂ cage [4a] to a solution or suspension of the appropriate metal nitrate salt in acetone at room temperature with vigorous stirring, as shown in Scheme 9A:

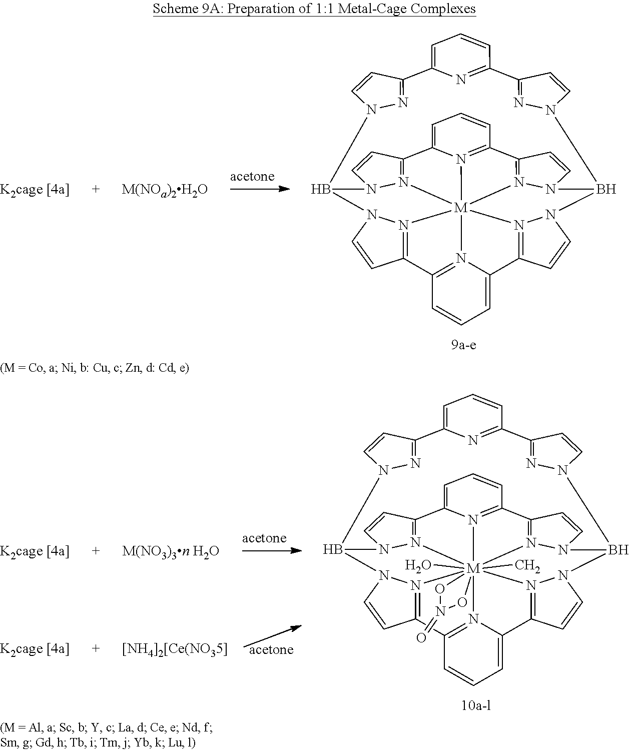

A general procedure that can be used for the preparation of metal complexes of the cage ligands described herein, and characterization of some specific examples, is described in Example 45 below.

Metals that have be shown to form at least mononuclear complexes with cage [4a]include. Li⁺, Na⁺, K⁺, Ag⁺, Co²⁺, Ni²⁺, Cu²⁺, Zn²⁺, Cd²⁺, Mg²⁺, Ca²⁺, Sr²⁺, Ba²⁺, Pb²⁺, Al³⁺, Ga³⁺, Sc³⁺, Y³⁺, La³⁺, Ce³⁺, Nd³⁺, Sm³⁺, Gd³⁺, Tb³⁺, Tm³⁺, Lu³⁺, Yb³⁺, and Hf⁴⁺. Many of these complexes have been characterized by ¹H NMR and ESI-MS and, in some cases, by solution phase magnetic measurements. Most of the complexes prepared so far appear to be air and moisture stable over a long period or indefinitely, although some may be hygroscopic. Some of the complexes are thermally sensitive and can be destroyed by mild heating at ca. 70° C., and the silver complexes tend to be light-sensitive. There was good evidence, from NMR titrations and magnetic measurements (Cu²⁺), that embodiments of the cage ligands described herein will bind up to three transition metal ions. In fact, several examples of complexes of ligand [4a] with three metal ions have been prepared by using more than 3.5 equivalents of the metal salt in the reaction, as shown in Scheme 9B:

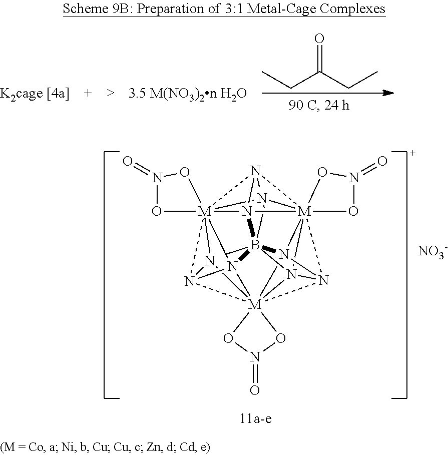

Table 4 lists examples of various metal complexes, including divalent, trivalent and tetravalent complexes, that have been prepared using the ligands described herein.

TABLE 4
Metal Complexes of Clathrochelate and Macrocyclic Ligands

| | | | Metal:Ligand |
|---|---|---|---|
| Complex | Metal | Ligand | Ratio |
| 9a | Co²⁺ | [4a] | 1:1 |
| 9b | Ni²⁺ | [4a] | 1:1 |
| 9c | Cu²⁺ | [4a] | 1:1 |
| 9d | Zn²⁺ | [4a] | 1:1 |
| 9e | Cd²⁺ | [4a] | 1:1 |
| 9f | Mg²⁺ | [4a] | 1:1 |
| 9g | Ca²⁺ | [4a] | 1:1 |
| 9h | Sr²⁺ | [4a] | 1:1 |
| 9i | Ba²⁺ | [4a] | 1:1 |
| 9j | Pb²⁺ | [4a] | 1:1 |
| 10a | Al³⁺ | [4a] | 1:1 |
| 10b | Sc³⁺ | [4a] | 1:1 |
| 10c | Y³⁺ | [4a] | 1:1 |
| 10d | La³⁺ | [4a] | 1:1 |
| 10e | Ce³⁺ | [4a] | 1:1 |
| 10f | Nd³⁺ | [4a] | 1:1 |
| 10g | Sm³⁺ | [4a] | 1:1 |
| 10h | Gd³⁺ | [4a] | 1:1 |
| 10i | Th³⁺ | [4a] | 1:1 |
| 10j | Tm³⁺ | [4a] | 1:1 |
| 10k | Yb³⁺ | [4a] | 1:1 |
| 10l | Lu³⁺ | [4a] | 1:1 |
| 10m | Ga³⁺ | [4a] | 1:1 |
| 10n | Hf⁴⁺ | [4a] | 1:1 |
| 11a | Co²⁺ | [4a] | 2:1 |
| 11b | Ni²⁺ | [4a] | 2:1 |
| 11c & 11c″ | Cu²⁺ | [4a] | 3:1 |
| 11c′ | Cu²⁺ | [4a] | 2:1 |
| 11d | Zn²⁺ | [4a] | 2:1 |
| 11e | Cd²⁺ | [4a] | 3:1 |
| 11f | Ag⁺ | [4a] | 8:2 |
| 12a | Cu²⁺ | [8a] | 1:1 |
| 12b | Y³⁺ | [8a] | 1:1 |

### Connector Chemistry for Tethering Boron-Canned Ligands to Biomolecules

To provide highly selective delivery of metal complexes of the boron-capped clathrochelate and macrocyclic ligands to targeted sites in vivo, the cage or ring structure can be tethered to a biomolecule such as a monoclonal antibody or a bioactive molecule of similar or different function. The cage and ring structures offer several sites for attachment of a connector that can link the ligand to the biomolecule. For the preparation of radiopharmaceuticals and radiotracers, generally it will be preferable to tether the cage or ring to the desired biomolecule, and then introduce a radioactive payload by forming a complex between the tethered ligand and a radioactive metal, thus forming an antibody-drug conjugate (ADC) which can be administered to a patient. An ADC is a highly targeted biopharmaceutical that comprises an active payload (e.g. a radionuclide), a connector, and a monoclonal antibody that selectively binds the ADC to specific antigens or receptors.

Several connectors (or “linkers”) for tethering a ligand to a biomolecule, such as a monoclonal antibody, have been described in the literature and many of these are commercially available. Most are small molecules with orthogonal bifunctional reactivity—that is to say, they react selectively at different ends with different things. The majority of the connectors are linked to the monoclonal antibody through either cysteine (Cys) SH or lysine (Lys) NH₂ groups present on the antibody. The attachment to the pre-drug (in this case the cage or ring ligand) is through complementary reactivity and can include various functional groups such as thioethers, disulphides, hydrazones, amides, esters, azides, alkynes and heterocycles.

Examples of how commercially available connectors can be used to tether a cage or ring ligand of the type described herein to a monoclonal antibody are illustrated in Scheme 10. Three monoclonal antibodies are shown, (8-1a) and (8-1c) having a pendant thiol group (SH), and (8-1b) having a pendant amine group (NH₂). The rest of the biomolecule is represented generically in each case. Similarly, three cage ligands are shown with (8-2a) having an amine group (NH₂), (8-2b) having a thiol group (SH), and (8-2c) having an aldehyde group. The rest of the cage ligand is represented generically in each case. Scheme 10 shows how cage ligand (8-2a) can be tethered to antibody (8-1a) by reaction with maleimidocaproyl, as a connector, to form cage-antibody molecule (8-3a). Similarly, Scheme 10 shows how cage ligand (8-2b) can be tethered to antibody (8-1b), again by reaction with maleimidocaproyl, to form cage-antibody molecule (8-3b); and how cage ligand (8-1c) can be tethered to antibody (8-2c) by reaction with another connector, to form cage-antibody molecule (8-3c). Similar chemistry could be used to tether a ring ligand to a monoclonal antibody.

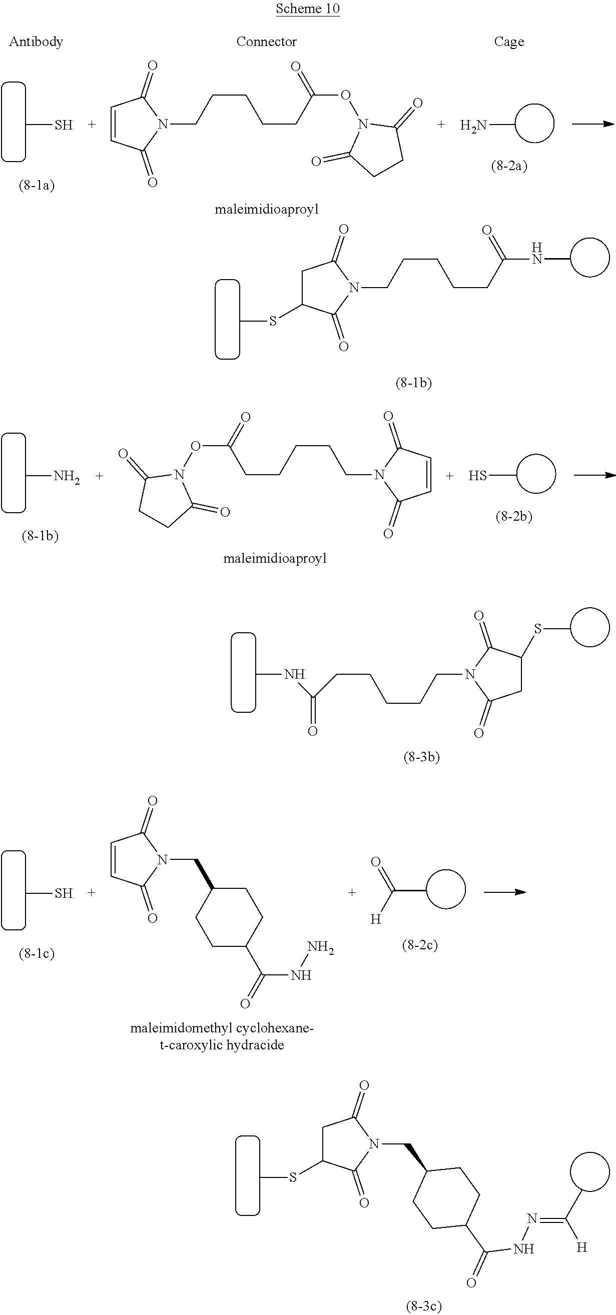

The above reactions can be performed in a stepwise manner. For example, in some cases the connector is first linked to the antibody and then the antibody-connector unit is linked to the cage ligand. In a subsequent step a metal radionuclide may be loaded into the cage ligand by formation of a metal complex, for example, by a metal displacement reaction. In other cases the connector is already attached to the cage, then the metal is introduced into the cage before the antibody is attached. This latter approach may be preferable as it may avoid metal uptake in other parts of the protein, rather than in the cage. Also the kinetics of metal uptake are likely to be faster if the protein is not already attached to the cage ligand.

As discussed above, the reactive functional groups on the cage or ring ligands can be incorporated in various ways and at various locations. For example, these groups (or other suitable reactive groups such as carboxylic acids, ketones and alkynes/azides for “click-chemistry”), can be directly attached at the 4-position of a pyridine ring in the arms of the ligand. They could also be separated from the pyridine ring in the arms by a suitable spacer moiety. In some embodiments, metal-catalysed cross-coupling reactions may be employed to introduce the chemically reactive group utilizing appropriate protecting group strategies. In some embodiments, installation of a desired functional groups may be achieved by metalation of bromo-substituents followed by quenching with an appropriate electrophile. In some embodiments, the 4-Br group in a 4-Br cage is replaced with reactive units such as alkenes via substitution with a thiolate (SR) or alkoxide (OR) group. With an alkene substituent in place, cross metathesis chemistry can be used to install groups such as alpha, beta-unsaturated esters, ketones and linked maleimides, as shown for example in Scheme 11. Such groups are expected to react directly with cysteine-SH groups.

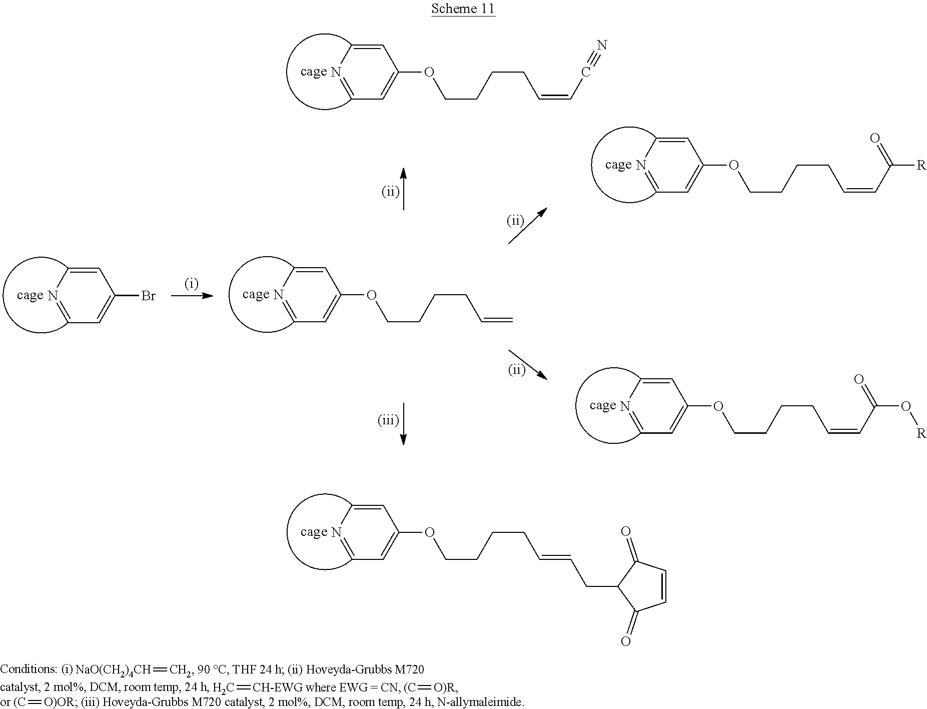

### Medical Applications of Clathrochelate and Macrocyclic Ligands, Complexes and ADCs

The boron-capped ligands described herein offer many attractive features for the construction of targeted, metal ion-based diagnostic imaging and therapeutic agents for use in medicine. A therapeutic agent is an agent which provides treatment for a disease or medical condition. A specific therapeutic agent may improve at least one symptom or parameter of a particular disease or medical condition. For instance, in tumor therapy, a therapeutic agent may reduce the size of a tumor, inhibit or prevent growth or metastases of a tumor, or eliminate a tumor.

Criteria for metal-containing ligands species as safe and effective diagnostic and therapeutic agents can include some or all of the following:
ability to tightly coordinate with radionuclides that are suitable imaging agents or anti-tumor agents;
high thermodynamic stability (large stability constant) to prevent or reduce tendency for metal loss in vivo;
low kinetic lability to prevent or reduce tendency for exchange with endogenous metal ions (including but not limited to Ca²⁺, Fe²⁺, Mg²⁺, Zn²⁺, Na⁺, K⁺);
organ or cell specificity (for example by attachment of monoclonal antibodies or similar type proteins, drug delivery by the use of extracellular vesicles e.g. exosomes, or the use of nanoparticles and the like); and/or
low off-target toxicity.

The ability of a ligand to hold radioactive metals with high stability under physiological conditions is important so that radiation damage to non-target cells can be reduced or avoided.

The cage and ring structures of embodiments of the boron-capped clathrochelate and macrocyclic ligands described herein offer a multidentate coordination environment. Pentadentate (five) coordination is generally regarded as the minimum needed to provide sufficiently large stability constants to prevent significant metal dissociation in vivo. The boron-capped clathrochelate ligands described herein have a minimum of six cage nitrogen donor atoms that can coordinate to the metal ion(s), and in some cases have nine cage nitrogen donor atoms that can coordinate to the metal ion(s). For example, for ligands with a pyridine link and a pair of pyrazole rings in each arm, there are up to nine nitrogen donors available for metal binding in the clathrochelate ligands, and up to six nitrogen donors available for metal binding in the macrocyclic ligands. As a result, embodiments of the metal complexes described herein are expected to have stability constants appropriate for use in vivo. Additionally, metal complexes of boron-capped clathrochelate ligands described herein with first row transition metal (Cu²⁺, Co²⁺) and with late lanthanide (Er³⁺, Yb³⁺) ions, have demonstrated very slow kinetic metal ion exchange between coordinated and free metal ions in solution (acetone-water or methanol-water), suggesting that their kinetic lability is low. As described above, the present cage and ring structures also lend themselves to modification at several positions: for example, on the pyrazole rings in the arms (e.g. substituent Q or J in Formula I and Formula VI), and on the linking pyridine ring in the arms (e.g. substituent Y in Formula I and Formula VI). Incorporation of suitable substituents at one or more of these sites can allow for the modification of physical properties of the ligands and their complexes, such as solubility and lipophilicity. Chemically reactive groups may also be introduced at one or more of these sites. Such chemically reactive groups may then be used to attach monoclonal antibodies, either directly attached or through a connector unit as discussed above.

An additional feature of some embodiments of the boron-capped clathrochelate ligands described is that they can bind as many as three metal ions at a time between adjacent cage arms, all in sites with at least six bound nitrogen donor atoms. This increases the metal ion payload, potentially allowing for lower overall effective doses of the therapeutic agent. It is also possible that different metal ions can be bound to the same cage ligand at the same time allowing simultaneous delivery of different therapeutic (e.g. α or β-emitters) modalities, or combined diagnostic and therapeutic (e.g. a β+ or γ emitter combined with an α or β-emitter) modalities.

Another attribute of the technology described herein is the speed and ease with which a desired radionuclide-ligand complex or radionuclide ADC can be prepared. A range of complexes can be synthesized in a one-pot, one-step process whereby a radioactive metal ion of choice (for example, as an aqueous solution of a salt of the radioactive metal) is simply added to a “kit” vial containing a ligand-antibody molecule (for example, also in saline solution) to produce the desired radiopharmaceutical, usually without need for purification or other subsequent processing. The resulting radiopharmaceutical would typically be injected into the patient, at a suitable dose, for diagnostic or therapeutic purposes. Depending on the half-life of the radionuclide and the application, in some cases the introduction of the radionuclide may need to be done immediately prior to injection.

### Use as Targeted Imaging and Therapeutic Agents for Nuclear Medicine

### Positron Emission Tomography (PET) Imaging:

⁸⁹Zr (half-life 78.4 hours) is a β⁺ emitter frequently used in PET imaging. As an electropositive metal with a 4+ionic charge, Zr would be suited to bind strongly with the multidentate nitrogen array provided by the cage or ring structures described herein. Its long half-life makes it suitable for targeted imaging using monoclonal antibodies linked to the ligand by a connector unit. Similarly, ⁶⁴Cu is another β⁺ emitter with a suitable half-life for PET imaging (12.7 hours). A metal complex [11c] of a boron-capped clathrochelate ligand with three bound Cu²⁺ ions has already been prepared and characterized as described herein (see Scheme 9B). The high radionuclide payload has the potential to provide enhanced imaging capabilities. Another approach is to incorporate a radionuclide into the ligand itself, instead of or as well as, preparing a radioactive metal complex of the ligand. For example, a cage or ring structure employing a halogen-based β⁺ emitter suitable for PET imaging (such as, but not limited to ¹²⁴I, half-life 4.2 days, or ⁷⁶Br, half-life 16.2 hours) could be prepared. It has been shown that embodiments of the ligands described herein can undergo extremely rapid electrophilic bromination or iodination at the 4′-position of the six pyrazole rings in the arms of the cage, with up to six bromine or iodine atoms introduced into the ligand. If a therapeutic radionuclide based on an α or β⁻ emitting metal isotope (see below) is then introduced, the resulting complex could function as both a targeted imaging and therapeutic agent.

### Therapeutic β⁻ Emitters:

There are several β⁻ emitters with suitable energies and half-lives for antibody-targeted radiotherapy that may be bound to the cage and/or ring ligands described herein. The most prominent of these β⁻ emitters are ⁹⁰Y (64 hours), ¹⁷⁷Lu (160 hours), ¹⁵³Sm (46 hours) and ¹⁶⁶Ho (27 hours). Yttrium complexes of boron-capped clathrochelate and macrocyclic ligands have been prepared and characterized as described herein. Additionally, it has been shown by electrospray ionization mass spectrometry that similar cage complexes can be prepared with virtually the entire lanthanide series, demonstrating that complexes of the present cage and ring structures and these metals can be prepared. Besides these metals, ⁶⁴Cu (12.7 hours) is a β⁻ emitter and, as mentioned above, it has been shown that cage [4a]will bind up to three Cu²⁺ ions, to make complex [11c], potentially making this a highly potent therapeutic agent.

Therapeutic α emitters:

Metal complexes of the present cage and ring ligands with ²²⁵Ac (10 days) and ²²⁷Th (18.5 days) are of great interest in cancer radiotherapy because not only do they decay by a-emission, but their decay chain involves further a-emissions resulting in a high radiation dose per decaying atom. The coordination chemistry of these ions elements is generally similar to non-radioactive Y³⁺, which has been demonstrated to form stable metal complexes with embodiments of the boron-capped clathrochelate and macrocyclic ligands described herein.

### Gamma Imaging Agents:

The historically important y-imaging radionuclides, ⁶⁷Ga (78 hours) and ¹¹¹In (67 hours), share many similarities in coordination chemistry with yttrium and the lanthanides and as such are very well-suited to forming stable metal ion complexes with embodiments of the boron-capped clathrochelate and macrocyclic ligands described herein.

Thus, the present technology provides specific structural elements, molecular designs, and methods of preparation that can be used to produce an expansive array of bioactive compounds for potential diagnostic, pharmaceutical and other uses. In essence, the present technology constitutes a toolbox that can be employed to produce a myriad of diagnostic and/or pharmaceutically active agents.

As mentioned above, previously known boron-containing cage structures are the borate-capped tris(oxaminate) ligands that contain ZB—O₃ capping groups with different oxaminate arms. These existing diborate-tris(oxaminate) cages differ significantly from the tris(pyrazolyl) boron-capped cages described herein. For example, the existing diborate-tris(oxaminate) cages are formed around the metal via a metal-templated synthesis, and generally the metal cannot be removed or changed without at least partially breaking the surrounding cage structure. Also, the cage itself is much smaller in size and it appears that it can generally only accommodate a first-row transition metal, with most of the reported examples containing iron or cobalt. The diborate-tris(oxaminate) cages are generally only capable of binding one metal in a pseudo-octahedral environment, and it seems that other small molecule guests do not bind within the cavity. There are some known related tris(oxaminate) cages based on different capping groups including Z₃GeO₃, Cl₃SnO₃ and Z₃SbO₃ but these capping units are more labile and the cages have similar properties to the ZB—O₃ versions. Embodiments of the boron-capped clathrochelate and macrocyclic ligands and their complexes described herein offer multiple advantages in biomedical applications, relative to known ligand systems.

Exemplary embodiments of the technology are illustrated by the following non-limiting Examples, in which the reactions were carried out under air or nitrogen as indicated using commercially available reagents as received unless otherwise specified. NMR spectra were recorded on Bruker AVANCE 300, 360 or 500 MHz instruments relative to residual solvent resonances (¹H, ¹³C), an external reference (BF₃·OEt₂ for ¹¹B) or by indirect detection (¹⁵N by HMBC, in ppm relative to NH₃). Melting points were determined using an Omega Engineering Model 199 instrument and are uncorrected. Mass spectra were recorded on a Q-TOF II instrument by MicroMass using electrospray ionization. TGA were recorded between room temperature and 600° C. Elemental analysis for C, H and N were obtained by Canadian Microanalytical Services, Delta, B.C., Canada or Guelph Chemical Laboratories Ltd., Guelph, ON, Canada.

### Example 1—2-(pyrazol-3′-yl)-6-(pyrazol-5′-yl)pyridine [3a]

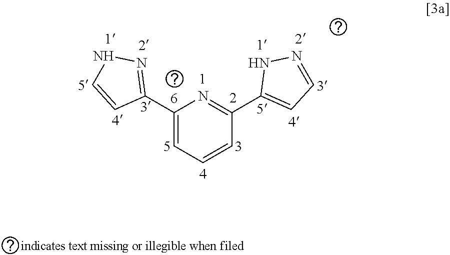 [SMILES: c1cc(-c2cc[nH]n2)nc(-c2ccn[nH]2)c1]

(General numbering scheme for compounds with this linkage is shown here)

Arm [3a] was prepared according to Scheme 1 as follows:

### 2,6-((N-dimethylamino)-(prop-2-en-1-one))-pyridine [2a]

A 250 mL round bottom flask was charged with 4.99 g (30.6 mmol) 2,6-diacetylpyridine [1a] and 150 mL toluene. To this solution was added 12.0 mL (90.3 mmol, 3 equiv) of N,N-dimethylformamide dimethyl acetal and the reaction mixture refluxed for 24 h. The reaction flask was transferred to the fridge (5° C. The precipitated solid was isolated by filtration and washed with hexane to yield 5.84 g (21.4 mmol, 69% yield) of [2a] as orange crystals. Mp. 220-225° C. ¹H NMR (300 MHz, CDCl₃): δ 8.22 (2H, d, ³J_{HH}=7.8 Hz, 3,5-pyH), 7.93 (2H, d, ³J_{HH}=12.9 Hz, N—CH═), 7.91 (1H, t, ³J_{HH}=7.8 Hz. 4-pyH), 6.62 (2H, d, ³J_{HH}=12.9 Hz, N—CH═CH), 3.19 (6H, br s, NCH₃), 3.00 (6H, br s, NCH₃); ¹³C{¹H} NMR (75 MHz, CDCl₃): δ 187.1 (C═O), 154.9 (CH═CHN), 154.78 (2,6-pyC), 137.72 (4-pyCH), 123.98 (3,5-pyCH), 91.76 (CH═CHN), 37.40 (NMe). Elem. analysis calcd. For C₁₅H₁₉N₃O₂: C, 65.91; H, 7.01; N, 15.39%. Found: C, 65.55; H, 6.98; N, 15.11%.

### 2-(pyrazol-3′-yl)-6-(pyrazol-5′-yl)pyridine [3a]

A 500 mL round bottom flask was charged with a 13.58 g (49.7 mmol) of [2a] and 250 mL anhydrous ethanol. Hydrazine monohydrate was added (7.40 mL, 153 mmol, 3 equiv) and the mixture refluxed at 80° C. for 48 hours followed by cooling. The product was isolated by vacuum filtration and washed with cold anhydrous ethanol to yield 6.09 g (28.8 mmol, 58%) of [3a] as white, needle-like crystals. Mp. 235-240° C. ¹H NMR (500 MHz, dmso-d₆): δ 13.50 (1H, br s, NH), 13.04 (1H, br s, NH), 7.62-7.87 (5H, m, 5′-pzH and pyH), 6.94-7.25 (2H, br m, 4′-pzH). ¹³C{¹H} NMR (125 MHz, dmso-d₆) δ 151.9, 151.2 (3′-pzC), 147.2, 141.1 (2,6-pyC), 140.3, 138.7 (5′-pzCH), 129.7 (4-pyCH), 118.2, 117.7 (3,5-pzCH), 103.5, 103.2 (4′-pzCH). Elem. analysis calcd. for C₁₁H₉N₅: C, 62.55; H, 4.29; N, 33.16%. Found: C, 61.99; H, 4.36; N, 32.98%.

### Example 2—2,6-di(pyrazol-3′-yl)-4-t-butylpyridine [3aa]

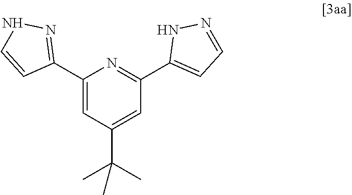 [SMILES: CC(C)(C)c1cc(-c2cc[nH]n2)nc(-c2ccn[nH]2)c1]

Arm [3aa] was prepared according to Scheme 1 as follows:

### 2,6-diacetyl-4-t-butylpyridine [1aa]

Commercial 4-t-butylpyridine (AK Scientific, 9.23 g, 68.3 mmol) was dissolved in 500 mL 0.4M H₂SO₄ in a 1 L flask with rapid stirring. Pyruvic acid (12.0 g, 137 mmol, 2 equiv) and AgNO₃ (1.1 g) were added and the solution allowed to stir for 15 minutes. Solid ammonium persulphate (48.0 g, 210 mmol) was added in portions. The reaction mixture was stirred at room temperature and worked up following the procedure detailed below in Example 7 for [3f]. ¹H NMR showed that the crude product contained the desired 2,6-diacetyl isomer as well as some 2-acetyl-4-t-butylpyridine. The isomers were separated by column chromatography on 60-mesh silica gel using 65:35 hexane:diethyl ether as eluent. 2,6-Diacetyl-4-t-butylpyridine eluted first. Yield: 1.46 g (9.7%). ¹H NMR (300 MHz, CDCl₃): δ 8.21 (2H, s, 3,5-pyH), 2.77 (6H, s, C(═O)CH₃), 1.36 (9H, s, CMe₃); ¹³C{¹H} (75 MHz, CDCl₃): δ 200.10 (C═O), 162.84 (4-pyC), 153.00 (2,6-pyC), 121.91 (3,5-pyCH), 35.54 (CMe₃), 30.61 (CMe₃), 25.84 (C(═O)CH₃).

### 2,6-((N-dimethylamino)-(prop-2-en-1-one))-4-t-butylpyridine [2aa]

A 50 mL round bottom flask was charged with 0.90 g (4.1 mmol) 2,6-(diacetyl)-4-t-butylpyridine [2aa] and 10 mL toluene. To this solution was added 1.6 mL (12.3 mmol, ca. 3 equiv) of N,N-dimethylformamide dimethyl acetal and the reaction mixture refluxed for 24 h. The reaction flask was transferred to the fridge (5° C.) to precipitate the product. The solid was isolated by filtration and washed with hexane to yield 1.31 g (3.98 mmol, 97% yield) of [2aa] as yellow-orange crystals. Mp. 180-183° C. ¹H NMR (300 MHz, CDCl₃): δ 8.21 (2H, s, 3,5-pyH), 7.88 (2H, d, ³J_{HH}=12.8 Hz, N—CH═), 6.59 (2H, d, ³J_{HH}=12.8 Hz, N—CH═CH), 3.15 (6H, br s, NCH₃), 2.96 (6H, br s, NCH₃), 1.34 (9H, s, CMe₃); ¹³C{¹H} NMR (75 MHz, CDCl₃): δ 187.36 (C═O), 161.79 (CH═CHN), 154.80 (2,6-pyC), 154.39 (4-pyC), 120.77 (3,5-pyCH), 91.83 (CH═CHN), 45.15 (NMe), 37.20 (NMe), 35.35 (CMe₃), 30.69 (CMe₃).

### 2,6-di(pyrazol-3′-yl)-4-t-butylpyridine [3aa]

A 50 mL round bottom flask was charged with 1.21 g (3.67 mmol) of [2aa] and 25 mL anhydrous ethanol. Hydrazine monohydrate was added (1.0 mL, 20.3 mmol, ca. 5.5 equiv) and the mixture refluxed at 80° C. for 48 hours followed by cooling. The product was isolated by vacuum filtration and washed with cold anhydrous ethanol to yield 0.89 g (3.33 mmol, 91%) of [3aa] as white, crystals. Mp. 188-190° C. ¹H NMR (300 MHz, dmso-d₆): δ 13.46 (1H, br s, NH), 13.02 (1H, br s, NH), 7.81 (2H, br s, 3- and 5-pyH), 7.64 (2H, br s, 5′-pzH), 7.04 (2H, br s, 4′-pzH), 1.36 (9H, s, CMe₃); ¹³C{¹H} NMR (75 MHz, dmso-d₆) δ 162.28, 160.76 (3′- and 3″-pzC or 2- and 6-pyC), 151.97, 151.41 (3′- and 3″-pzC or 2- and 6-pyC), 147.25 (4-pyC), 141.43, 140.29 (5′ and 5″-pzCH), 129.69 (4″-pzCH), 115.28, 114.64 (3- and 5-pyCH), 103.62 (4′-pzCH), 34.13 (CMe₃), 30.27 (CMe₃).

### Example 3—2,6-di(pyrazol-3′yl)-4-chloropyridine [3b]

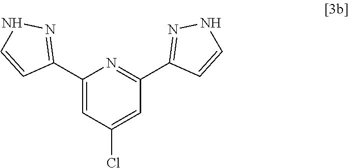 [SMILES: Clc1cc(-c2cc[nH]n2)nc(-c2cc[nH]n2)c1]

Arm [3b] was prepared according to Scheme 1 as follows:

### 2,6-(diacetyl)-4-chlororopyridine [1b]

The synthesis of [1b]followed the procedure of Nocera et al, J. Org. Chem. 2017, 82, 12933, with some modifications. Commercial 4-choloropyridine hydrochloride (ChemImpex Ltd., 50.00 g, 333.3 mmol) was dissolved in 400 mL water and the solution pH was adjusted to 9 with saturated sodium carbonate solution. The basic mixture was extracted with 3×250 mL CH₂Cl₂ and the combined CH₂Cl₂ extracts were then extracted using 0.5M H₂SO₄ (3×500 mL). The H₂SO₄ extracts were added to a 3 L flask. Pyruvic acid (94 mL, 1.34 mol, 4 equiv) and AgNO₃ (5.63 g, 33.33 mmol, 10 mol %) were added and the solution allowed to stir for 15 minutes. Solid sodium persulphate (317 g, 1.33 mol, 4 equiv) was added in large portions (caution: there is an induction period of a few minutes before CO₂ gas evolution begins) with care. The resulting brown suspension was stirred overnight at room temperature and then charged with further pyruvic acid (47 mL, 0.67 mmol, 2 equiv) and silver nitrate (2.83 g, 16.7 mmol). Additional Na₂S₂O₈ (158 g, 0.67 mmol, 2 equiv) was added with care. The resulting suspension was allowed to stir a further 48 h. The reaction mixture was filtered through a 3 inch silica plug to yield a red-orange acidic water phase. The silica plug was washed with CH₂Cl₂ (ca. 400 mL) and this was set aside. The acidic filtrate was carefully made basic with concentrated NaOH solution and the mixture turned deep red-brown with some solids appearing. Once pH 10 was reached, no further gas evolution was observed and the solution was allowed to cool to room temperature. Extraction with CH₂Cl₂ (3×1200 mL) gave a deep orange solution that was combined with the silica plug eluent, dried over MgSO₄ and filtered through a 3 inch silica gel (60 mesh) plug. The filtrate was golden yellow. The solvent was removed under reduced pressure to yield an oily residue that contained the desired product as the major component by ¹H NMR. The residue was dry loaded onto a silica column. Elution with pure CH₂Cl₂ produced pure product between 500-2000 mL eluent volume. Yield: 7.00 g (7%). ¹H NMR (300 MHz, CDCl₃): δ 8.04 (2H, s, 3,5-pyH), 2.69 (s, 6H, C(═O)CH₃); ¹³C{¹H} (75 MHz, CDCl₃): δ 197.88 (C═O), 153.81 (2,6-pyC), 146.77 (4-pyC-Cl), 124.77 (3,5-pyC), 25.53 (CH₃).

### 2,6-((N-dimethylamino)-(prop-2-en-1-one))-4-choloropyridine [2b]

A 500 mL round bottom flask was charged with 6.73 g (34.1 mmol) 2,6-(diacetyl)-4-chloropyridine [1b] and 200 mL toluene. To this solution was added 13.4 mL (102 mmol, ca. 3 equiv) of N,N-dimethylformamide dimethyl acetal and the reaction mixture refluxed for 48 h. The reaction mixture was cooled and the orange precipitate was filtered off by suction filtration. Yield: 6.25 g (20.3 mmol, 60% yield). Mp. dec. 275° C. ¹H NMR (500 MHz, CDCl₃): δ 8.19 (2H, s, 3,5-pyH), 7.91 (2H, d, ³J_{HH}=12.5 Hz, N—CH═CH), 6.53 (2H, br d, ³J_{HH}=12.5 Hz, N—CH═CH), 3.20 (6H, br s, NCH₃), 2.99 (6H, br s, NCH₃); ¹³C{¹H} NMR (125 MHz, CDCl₃): δ 185.44 (C═O), 156.27 (2,6-pyC), 154.94 (CH═CHNMe₂). 146.26 (4-pyC), 123.87 (3,5-pyC), 91.31 (CH═CHNMe₂), 45.34 (NMe), 37.31 (NMe).

### 2,6-di(pyrazol-3′-yl)-4-chloropyridine [3b]

A 250 mL round bottom flask was charged with 6.25 g (20.3 mmol) of [2b] and 250 mL anhydrous ethanol. Hydrazine monohydrate was added (3.0 mL, 62 mmol, ca. 3 equiv) and the mixture refluxed at 80° C. for 24 hours followed by cooling in the freezer. The product was isolated by vacuum filtration to yield 3.35 g (13.6 mmol, 67%) of [3b] as white, mat-like needles. The NMR spectra showed a mixture of the symmetrical 2′-NH/2″-NH tautomer (shown above) and the unsymmetrical 2′-NH/1″-NH isomer (denoted by *) in approximately 50:50 ratio. Mp.>320° C. ¹H NMR (500 MHz, dmso-d₆): δ 13.58 (1H, br s, NH*), 13.19 (3H, br s, overlapping NH and NH*), 7.88 (5H, br s, overlapping pyH, pyH* and pzH), 7.63 (1H, br s, pzH*), 7.27 (1H, br s, pzH*), 7.12 (1H, br s, pzH*), 6.99 (2H, br s, pzH). ¹³C{¹H} NMR (125 MHz, dmso-d₆) δ 153.70 (2,6-pyC), 149.04 (2,6-pyC), 143.77 (br, pzC), 140.41 (4-pyC-Cl), 140.27 (br, 4-pyC-Cl), 130.19 (5′-pzCH), 117.97 (br, 3,5-pyCH), 117.12 (3,5-pyCH), 104.58 (4′-pzCH), 103.88 (br, 4′-lpzCH).

### Example 4—2,6-di(pyrazol-3′-yl)-4-bromopyridine [3c]

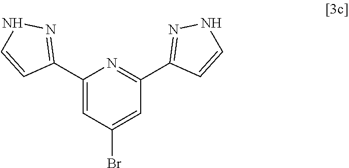 [SMILES: Brc1cc(-c2cc[nH]n2)nc(-c2cc[nH]n2)c1]

Arm [3c] was prepared according to Scheme 1 as follows:

### 2,6-(diacetyl)-4-bromopyridine [1c]

Following the procedure of Nocera et al, J. Org. Chem. 2017, 82, 12933, commercial 4-bromopyridine hydrochloride (ChemImpex Ltd., 50.00 g, 257.1 mmol) was dissolved in 400 mL water and the solution pH was adjusted to 9 with saturated sodium carbonate solution. The basic mixture was extracted with 3×250 mL CH₂Cl₂ and the combined CH₂Cl₂ extracts were then extracted using 0.5M H₂SO₄ (3×350 mL). The H₂SO₄ extracts were added to a 3 L flask equipped with a stir bar. Pyruvic acid (91.0 g, 1.03 mol, 4 equiv) and AgNO₃ (4.4 g, 25.7 mmol, 10 mol %) were added and the solution allowed to stir for 15 minutes. Solid sodium persulphate (184 g, 775 mmol, 3 equiv) was added all in one portion (caution: there is an induction period of a few minutes) with care. The resulting brown suspension was stirred for 48 h at room temperature. The reaction mixture was filtered to remove silver salts and the filtrate carefully made basic with concentrated NaOH solution. Washing the filter cake with dichloromethane afforded a high purity product. Extraction of the solution as described below affords about 20% more product but it is contaminated with the monoacetyl product.

The reaction mixture turned deep red-brown and became warm during basification. Once pH 10 was reached, no further gas evolution was observed, and the solution was allowed to cool to room temperature. Extraction with CH₂Cl₂ (3×400 mL) gave a deep orange solution that was dried over MgSO₄ and filtered through a 3 inch silica gel (60 mesh) plug. The filtrate was initially yellow in color but changed to deep orange within minutes. The solvent was removed and the oily residue dry loaded onto a silica column (30×2 inches). Elution with pure CH₂Cl₂ produced pure product between 600-1100 mL eluent volume. Fractions between 1100 and 1400 mL contained some product and 2-acetyl-4-bromopyridine while the fraction eluting between 1400 and 1700 mL contained pure 2-acetyl-4-bromopyridine. Yield: 13.9 g (18%). ¹H NMR (300 MHz, CD₂Cl₂): δ 8.33 (2H, s, 3,5-pyH), 2.75 (s, 6H, C(═O)CH₃); ¹³C{¹H} (75 MHz, CD₂Cl₂): δ 198.53 (C═O), 154.14 (2,6-pyC), 135.72 (4-pyC), 128.28 (3,5-pyCH), 25.99 (CH₃).

### 2,6-((N-dimethylamino)-(prop-2-en-1-one))-4-bromopyridine [2c]

A 250 mL round bottom flask was charged with 5.40 g (22.3 mmol) 2,6-(diacetyl)-4-bromopyridine [1c] and 80 mL toluene. To this solution was added 8.9 mL (67 mmol, ca. 3 equiv) of N,N-dimethylformamide dimethyl acetal and the reaction mixture refluxed for 24 h. The reaction flask was cooled to(5° C. to precipitate the product. The yellow-orange crystalline solid was isolated by filtration and washed with hexane. A deep orange second crop was obtained adding hexane to the filtrate above. The combined yield was 5.50 g of [2c] (15.6 mmol, 70% yield). Mp. dec. 240° C. ¹H NMR (500 MHz, CDCl₃): δ 8.35 (2H, s, 3,5-pyH), 7.91 (2H, d, ³J_{HH}=12.5 Hz, N—CH═CH), 6.52 (2H, br d, ³J_{HH}=12.5 Hz, N—CH═CH), 3.19 (6H, br s, NCH₃), 2.98 (6H, br s, NCH₃); ¹³C{¹H} NMR (125 MHz, CDCl₃): δ 185.34 (C═O), 155.92 (2,6-pyC), 154.92 (CH═CHNMe₂). 134.93 (4-pyC), 126.90 (3,5-pyC), 91.21 (CH═CHNMe₂), 45.33 (NMe), 37.29 (NMe).

### 2,6-di(pyrazol-3′-yl)-4-bromopyridine [3c]

A 250 mL round bottom flask was charged with 4.40 g (12.5 mmol) of [2c] and 100 mL anhydrous ethanol. Hydrazine monohydrate was added (3.0 mL, 37.5 mmol, ca. 3 equiv) and the mixture refluxed at 80° C. for 48 hours followed by cooling in the freezer. The product was isolated by vacuum filtration and washed with hexane to yield 3.45 g (11.9 mmol, 95%) of [3c] as white, voluminous crystals. Mp. >320° C. ¹H NMR (500 MHz, dmso-d₆): δ 13.28* (2H, br s, NH), 8.03 (2H, s, 3,5-pyH), 7.77 (d, ³J_{HH}=2 Hz, 5′-pzH), 7.07 (d, ³J_{HH}=2 Hz, 4′-pzH); * absent from the spectrum if the sample contains significant water. ¹³C{¹H} NMR (125 MHz, dmso-d₆): δ 151.73 (2,6-pyC), 146.3 (br s, 3′-pzC), 133.98 (br s, 5′-pzCH), 133.25 (4-pyC-Br), 120.36 (3,5-pyCH), 104.29 (4′-pzCH); * spectrum is sharper if the sample contains significant water.

### Example 5—2,6-di(pyrazol-3′-yl)-4-iodopyridine [3d]

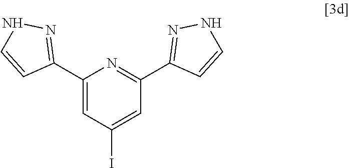 [SMILES: Ic1cc(-c2cc[nH]n2)nc(-c2cc[nH]n2)c1]

Arm [3d] was prepared according to Scheme 1 as follows:

### 2,6-(diacetyl)-4-iodopyridine [1d]

Compound [1d] was prepared by the procedure of Nocera et al, J. Org. Chem. 2017, 82, 12933, with some significant modifications. Commercial 4-iodopyridine (AK Scientific, 5.00 g, 24.4 mmol) was dissolved in 200 mL dichloromethane to afford a pale-yellow solution and an insoluble red solid that was filtered off and discarded. The dichloromethane phase was washed twice with 200 mL saturated aqueous NaHCO₃ and the extracted with 300 mL of 0.4 M aqueous H₂SO₄. The aqueous acid phase was drained directly into a 1 L round bottom flask and pyruvic acid (8.00 mL, 10.0 g, 114 mmol, 4.6 equiv) and AgNO₃ (1.80 g, 10.6 mmol, 46 mol %) were added to the solution. Solid ammonium persulphate (25.6 g, 112 mmol, 4.6 equiv) was added in large portions with care. The resulting suspension slowly turned gray-green and precipitate appeared. After 24 h, half of the above quantities again of pyruvic acid, silver nitrate and then ammonium persulphate were added to the suspension and stirring continued for a further day. The reaction mixture was neutralized with solid K₂CO₃ until the pH reached 10 and gas evolution ceased. Dichloromethane (300 mL) was added and the red-brown mixture was filtered through Celite to remove silver salts. The filter pad was washed with a further 500 mL dichloromethane and the organic phase separated, dried over anhydrous MgSO₄, filtered and the filtrate evaporated under reduced pressure to give a purple oil that solidified on standing. NMR showed that this residue was the desired 2,6-diacetyl-4-iodopyridine. The solid was redissolved in minimum dichloromethane and flash chromatographed on silica using dichloromethane to elute pure 2,6-diacetyl-4-iodopyridine, [1d]. Yield: 1.44 g (20%). Mp. 102-105° C. ¹H NMR (500 MHz, dmso-d₆): δ 8.430 (2H, s, 3,5-pyH), 2.689 (s, 6H, C(═O)CH₃); ¹³C{¹H} (125 MHz, dmso-d₆): δ 197.81 (C═O), 152.33 (2,6-pyC), 133.02 (3,5-pyCH), 108.11 (4-pyCI), 25.42 (CH₃).

### 2,6-di-((N-dimethylamino)-(prop-2-en-1-one))-4-iodopyridine [2d]

A 250 mL round bottom flask was charged with 0.995 g (3.44 mmol) 2,6-(diacetyl)-4-iodopyridine [1d] and 20 mL toluene. To this solution was added 1.4 mL (10.5 mmol, ca. 3 equiv) of N,N-dimethylformamide dimethyl acetal and the reaction mixture refluxed for 24 h. The orange crystalline product that precipitated directly from the reaction mixture on cooling was isolated by filtration and washed with hexane. A deep orange second crop was obtained adding hexane to the filtrate above. The combined yield was 0.800 g of [2d] (2.00 mmol, 58% yield). Mp. dec.>222° C. ¹H NMR (500 MHz, CDCl₃): δ 8.555 (2H, s, 3,5-pyH), 7.902 (2H, d, ³J_{HH}=12.7 Hz, N—CH═CH), 6.511 (2H, br d, ³J_{HH}=12.7 Hz, N—CH═CH), 3.185 (6H, br s, NCH₃), 2.976 (6H, br s, NCH₃); ¹³C{¹H} NMR (125 MHz, CDCl₃): δ 185.34 (C═O), 155.06 (2,6-pyC), 154.83 (CH═CHNMe₂). 132.86 (3,5-pyC), 107.39 (4-pyCI), 91.18 (CH═CHNMe₂), 45.32 (NMe), 37.29 (NMe).

### 2,6-di(pyrazol-3′-yl)-4-iodopyridine [3d]

A 250 mL round bottom flask was charged with 0.724 g (1.81 mmol) of [2d] and 15 mL anhydrous ethanol. Hydrazine monohydrate was added (0.30 mL, 6.2 mmol, ca. 3.4 equiv) and the mixture refluxed at 80° C. for 48 hours followed by cooling. The product was isolated by vacuum filtration and washed with hexane to yield 0.608 g (1.80 mmol, 99%) of [3d] as off-white crystals. Mp. dec. ~240° C. ¹H NMR (300 MHz, dmso-d₆): δ 13.20 (2H, v br s, NH), 8.21 (2H, br s, 3,5-pyH), 7.76 (2H, br s, 5′-pzH), 7.05 (2H, br s, 4′-pzH). ¹³C{¹H} NMR (125 MHz, dmso-d₆ with 4 drops D₂O): δ 153.0 (br s, 3′-pzC), 130.7 (br s, 5′-pzCH), 126.8 (3,5-pyCH), 107.7 (4-pyC-I), 104.7 (br s, 4′-pzCH); 2,6-pyC not observed.

### Example 6—2,6-di(pyrazol-3′-yl)-4-cyanomethylpyridine

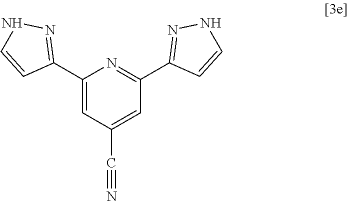 [SMILES: N#Cc1cc(-c2cc[nH]n2)nc(-c2cc[nH]n2)c1]

Arm [3e] was prepared according to Scheme 1 as follows:

### 2,6-(diacetyl)-4-cyanopyridine [1e]

The procedure below follows that of C. M. Weekly et al, Inorg. Chem. 2017, 56, 6076-6093. Commercial isonicotinonitrile (4-cyanopyridine) (Aldrich, 21.6 g, 210 mmol) was dissolved in 1050 mL 0.4M H₂SO₄ in a 3 L flask with rapid stirring. Pyruvic acid (55.8 g, 630 mmol, 3 equiv) and AgNO₃ (3 g, 18 mmol) were added, and the solution allowed to stir for 15 minutes. Solid ammonium persulphate (162 g, 720 mmol) was added in portions (caution: induction period of a few minutes after each addition). The reaction mixture was stirred at room temperature overnight. The reaction mixture was brought to a pH of ca. 10 using K₂CO₃ (ca. 200 g) and the resulting purple suspension was extracted with 3×500 mL of dichloromethane. The combined organic extracts were dried over MgSO₄ and filtered through a thick pad of silica and Celite, washing the solids with an additional 250 mL of CH₂Cl₂. Removal of the solvent yielded a white solid containing unreacted isonicotinonitrile, 2-acetyl-4-cyanopyridine and 2,6-diacetyl-4-cyanopyridine. Recrystallization from hexane and ethyl acetate removed the starting isonicotinonitrile but left a mixture of the acetylated pyridines. Column chromatography on silica using 33% ethyl acetate in hexane afforded early fractions containing both acetylated pyridines and a later fraction of pure 2,6-diacetyl-4-cyanopyridine. It is possible to remove most of the 2-acetyl-4-cyanopyridine by repeated recrystallization from hot ethyl acetate/hexane Yield: 8.9 g (23%). Mp. 90-95° C. ¹H NMR (300 MHz, CDCl₃): δ 8.41 (2H, s, 3,5-pyH), 2.81 (s, 6H, C(═O)CH₃); ¹³C{¹H} (75 MHz, CDCl₃): δ 197.38 (C═O), 150.17 (2,6-pyC), 128.21 (3,5-pyCH), 126.33 (CN), 123.51 (4-pyC-CN), 25.58 (CH₃).

### 2,6-((N-dimethylamino)-(prop-2-en-1-one))-4-cyanopyridine [2e]

A 100 mL Schlenk flask was charged with a 1.60 g (8.50 mmol) 2,6-(diacetyl)-4-cyanopyridine [1e] and 50 mL toluene. To this solution was added 3.4 mL (28.3 mmol, ca. 3 equiv) of N,N-dimethylformamide dimethyl acetal and the reaction mixture refluxed for 24 h. The reaction flask was transferred to the fridge (5° C.). The precipitated solid was isolated by suction filtration and washed with hexane to yield 1.78 g (5.97 mmol, 70% yield) of [2e] as ochre yellow crystals. Mp. dec. without melting ca. 250° C. ¹H NMR (300 MHz, CDCl₃): δ 8.39 (2H, s, 3,5-pyH), 7.94 (2H, d, ³J_{HH}=12.9 Hz, N—CH═), 6.50 (2H, br d, ³J_{HH}=12.9 Hz, N—CH═CH), 3.21 (6H, br s, NCH₃), 3.00 (6H, br s, NCH₃); ¹³C{¹H} NMR (75 MHz, CDCl₃): δ 184.37.1 (C═O), 155.96 (CH═CHN), 155.32 (2,6-pyC), 125.09 (3,5-pyCH), 122.01 (CN), 118.77 (4-pyC-CN), 90.69 (CH═CHN), 45.46 (NMe), 37.36 (NMe).

### 2,6-di(pyrazol-3′-yl)-4-cyanomethylpyridine [3e]

A 250 mL Schlenk flask was charged with 5.250 g (17.60 mmol) of [2e] and 150 mL anhydrous ethanol. Hydrazine monohydrate was added (1.75 mL, 36.1 mmol, ca. 2.05 equiv) and the mixture refluxed at 80° C. for 24 hours. Excess hydrazine should not be used, or it will react with the nitrile to afford an amidrazone. The pure off-white product precipitated from solution as the reaction proceeded. Vacuum filtration and washing with diethyl ether afforded [3e] as a mat-like off-white solid. Yield: 55%. Mp. 288-290° C. ¹H NMR (500 MHz, dmso-d₆): δ 13.68, 13.64, 13.27 (2H, br s, NH), 8.27, 8.20, 8.14 (2H, br overlapping s, 3,5-pyCH), 7.90, 7.66 (2H, br s, 5′-pzH), 7.33, 7.16, 7.11, 7.02 (2H, v br s, 4′-pzH); ¹³C{¹H} NMR (125 MHz, dmso-d₆) δ 153.05 (br s, 3′-pzC), 149.46 (br s, 2,6-pyC), 130.28 (br s, 5′-pzCH), 121.06 (br s, 4-pyC-CN), 119.16 (br s, 3,5-pyCH), 116.99 (sharp s, CN), 104.40 (br s, 4′-pzCH). The spectrum appearance varies dramatically depending on the amount of water present due to NH tautomerization. The spectral data listed above is for a sample dried under vacuum for 48 h at 120° C. ESI-MS, neg. mode: [Cl₂H₇N₆], m/z observed: 235 amu.

### Example 7—2,6-di(pyrazol-3′-yl)-4-trifluoromethylpyridine [3f]

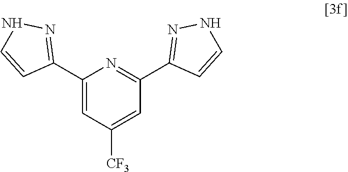 [SMILES: FC(F)(F)c1cc(-c2cc[nH]n2)nc(-c2cc[nH]n2)c1]

Arm [3f] was prepared according to Scheme 1 as follows:

### 2,6-(diacetyl)-4-trifluoromethylpyridine [1f]

Commercial 4-trifluoromethylpyridine (AK Scientific, 14.07 g, 95.6 mmol) was dissolved in 450 mL 0.4M H₂SO₄ in a 1 L flask with rapid stirring. Pyruvic acid (17.5 g, 200 mmol, 2.1 equiv) and AgNO₃ (1.2 g, 7.1 mmol) were added and the solution allowed to stir for 15 minutes. Solid ammonium persulphate (64.0 g, 296 mmol) was added in portions (caution: induction period of a few minutes after each addition). The reaction mixture was stirred at room temperature overnight and then another 8.75 g of pyruvic acid followed by an additional 32.0 g of NH₄S₂O₈ was added and the resulting mixture stirred for 2 further days at room temperature. The reaction mixture was extracted with 2×400 mL of dichloromethane and the combined organic extract was washed with saturated NH₄Cl (2×400 mL) and water (2×400 mL). The organic phase was dried over anhydrous MgSO₄, filtered and the filtrate evaporated under reduced pressure to a pale yellow oil that solidified on standing. ¹H NMR showed that the crude product contained ca. 75% of the desired 2,6-diacetyl isomer, 23% of the 2,5-diacetyl product and 2% 2-acetyl-4-trifluoromethylpyridine. The isomers were separated by column chromatography on 60-mesh silica gel using 65:35 hexane:diethyl ether as eluent. 2,6-Diacetyl-4-trifluoromethylpyridine elutes first followed by the 2,5-isomer and finally by the yellow 2-acetyl-4-trifluoromethylpyridine. Yield: 11.09 g (50.2%). ¹H NMR (500 MHz, CDCl₃): δ 8.41 (2H, s, 3,5-pyH), 2.80 (s, 6H, C(═O)CH₃); ¹³C{¹H} (125 MHz, CDCl₃): δ 197.8 (C═O), 154.0 (2,6-pyC), 141.0 (q, ²J_{CF}=35 Hz, 4-pyC-CF₃), 122.2 (q, ¹J_{CF}=273 Hz, CF₃), 120.5 (d, ³J_{CF}=3 Hz, 3,5-pyCH), 25.6 (CH₃); ¹⁹F NMR (282.5 MHz, CDCl₃): δ −64.77. Elem. analysis calcd. for C₁₀H₈F₃NO₂: C, 51.96; H, 3.49; N, 6.06%. Found: C, 52.11; H, 3.60; N, 5.89%.

### 2,6-((N-dimethylamino)-(prop-2-en-1-one))-4-trifluoromethylpyridine [2f]

A 250 mL round bottom flask was charged with 11.00 g (47.58 mmol) 2,6-(diacetyl)-4-trifluoromethylpyridine [1f] and 150 mL toluene. To this solution was added 20.0 mL (150 mmol, ca. 3 equiv) of N,N-dimethylformamide dimethyl acetal and the reaction mixture refluxed for 24 h. The reaction flask was transferred to the fridge (5° C.) to precipitate the product. The solid was isolated by filtration and washed with hexane to yield 11.27 g (33.02 mmol, 69.4% yield) of [2f] as deep orange crystals. Mp. 161-163° C. ¹H NMR (300 MHz, CDCl₃): δ 8.44 (2H, s, 3,5-pyH), 7.97 (2H, d, ³J_{HH}=12.6 Hz, N—CH═), 6.57 (2H, d, ³J_{HH}=12.6 Hz, N—CH═CH), 3.22 (6H, br s, NCH₃), 3.01 (6H, br s, NCH₃); ¹³C{¹H} NMR (75 MHz, CDCl₃): δ 185.1 (C═O), 156.2 (CH═CHN), 155.1 (2,6-pyC), 140.1 (q, ²J_{CF}=35 Hz, 4-pyC-CF₃), 123.1 (q, ¹J_{CF}=274 Hz, CF₃), 119.3 (q, 3,5-pyCH, ³J_{CF}=3.2 Hz), 91.08 (CH═CHN), 45.37 (NMe), 37.31 (NMe); ¹⁹F (282.5 MHz, CDCl₃): δ −64.57. Elem. analysis calcd. for C₁₆H₁₈F₃N₃O₂: C, 56.30; H, 5.32; N, 12.31%. Found: C, 56.39; H, 5.55; N, 12.01%.

### 2,6-di(pyrazol-3′-yl)-4-trifluoromethylpyridine [3f]

A 500 mL round bottom flask was charged with 11.20 g (32.81 mmol) of [2f] and 250 mL anhydrous ethanol. Hydrazine monohydrate was added (7.5 mL, 152 mmol, ca. 4.6 equiv) and the mixture refluxed at 80° C. for 48 hours followed by cooling in the freezer. The product was isolated by vacuum filtration and washed with cold anhydrous ethanol to yield 7.25 g (26.0 mmol, 79%) of [3f] as white, crystals. Mp. 221-224° C. ¹H NMR (300 MHz, dmso-d₆): δ 8.08 (s, 3,5-pyH), 7.82 (d, ³J_{HH}=2.1 Hz, 5′-pzH), 7.14 (d, ³J_{HH}=2.1 Hz, 4′-pzH); ¹³C{¹H} NMR (75 MHz, dmso-d₆) δ 152.2 (2,6-pyC), 147.0 (br s, 3′-pzC), 138.4 (q, ²J_{CF}=33 Hz, 4-pyC-CF₃), 133.47 (br s, 5′-pzCHl), 123.1 (q, ¹J_{CF}=273 Hz, CF₃), 112.7 (q, 3,5-pyCH, ³J_{CF}=3.5 Hz), 104.3 (4′-pzCH); ¹⁹F (282.5 Hz, dmso-d₆): −63.70. Elem. analysis calcd. for C₁₂H₈F₃N₅: C, 51.62; H, 2.89; N, 25.08%. Found: C, 51.18; H, 3.02; N, 24.66%.

### Example 8a—2,6-di-(5′-methylpyrazol-3′-yl)pyridine [3′a]

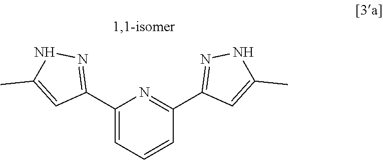

Arm [3′a] was prepared according to Scheme 5A as follows:

### 2,6-(3′-(N,N-dimethylamino)-(but-2-en-1-one))-pyridine [2′a]

A 100 mL Schlenk flask was charged with 2.05 g (12.6 mmol) 2,6-diacetylpyridine, [1a] and 30 mL toluene. To this solution was added 5.03 g (37.8 mmol, 3 equiv) of N,N-dimethylacetamide dimethyl acetal and the reaction mixture refluxed for 24 h. The reaction flask was transferred to the freezer (−10° C.) to precipitate the product. The solid was isolated by filtration and washed with hexane to yield 1.51 g (5.01 mmol, 40% yield) of [2′a] as yellow-orange crystals. Mp. 212-213° C. ¹H NMR (500 MHz, CDCl₃): δ 8.195 (2H, d, ³J_{HH}=7.8 Hz, 3,5-pyH), 7.850 (1H, t, ³J_{HH}=7.8 Hz, 4-pyH), 6.768 (2H, s, =CH), 3.056 (12H, s, NCH₃), 2.682 (6H, s, ═CCH₃); ¹³C{¹H} NMR (125 MHz, CDCl₃): δ 185.39 (C═O), 164.78 (CH═CMeN), 155.96 (2,6-pyC), 137.38 (4-pyCH), 123.02 (3,5-pyCH), 90.79 (CH═CMeN), 40.50 (NMe), 16.55 (CH═CMeN). Elem. analysis calcd. For C₁₇H₂₃N₃O₂: C, 67.75; H, 7.69; N, 13.94%. Found: C, 67.49; H, 7.72; N, 13.59%.

### 2,6-di-(5′-methylpyrazol-3′-yl)pyridine [3′a]

A 250 mL Schlenk flask was charged with 1.40 g (4.64 mmol) of [2′a] and 75 mL anhydrous ethanol. Hydrazine monohydrate was added (0.75 mL, 15.5 mmol, 3.3 equiv) and the mixture refluxed at 80° C. for 48 hours. On cooling a white precipitate formed that was isolated by vacuum filtration and washed with diethyl ether to yield 1.01 g (3.54 mmol, 76%) of [3′a] as white crystals of the mono ethanol solvate. Mp. 130-135° C. The ¹H NMR showed a 1:1:1 mixture of the symmetric 1,1-NH tautomer (shown in the figure), the 2,2-NH tautomer and the asymmetric 1,2-NH tautomer. Assignments that follow were made using COSY, HSQC and HMBC methods. ¹H NMR (500 MHz, dmso-d₆): δ 13.08 (2H, br s, 1,1-NH), 13.03 (1H, br s, 1,2-NH), 12.70 (1H, br s, 1,2-NH), 12.68 (2H, br s, 2,2-NH), 7.88 (1H, t, ³J_{HH}=7.8 Hz, 1,1-4-pyH), 7.80 (5H, m, 2,2-3,4,5-pyH overlapping 1,2-4,5-pyH), 7.63 (3H, d overlapping m, ³J_{HH}=7.8 Hz, 1,1-3,5-pyH and 1,2-3-pyH), 6.96 (1H, s, 1,2-4-pzH), 6.74 (2H, s, 1,1-4-pzH), 6.66 (3H, br overlapping s, 2,2-4-pzH and 1,2-4-pzH), 4.36 (1H, t, ³J_{HH}=5.0 Hz, CH₃CH₂OH), 3.45 (2H, m, CH₃CH₂OH), 2.31 (9H, br s, 2,2-Me and 1,2-Me), 2.25 (9H, br s, 1,1-Me and 1,2-Me), 1.06 (3H, t, ³J_{HH}=7.0 Hz, CH₃CH₂OH). ¹³C{¹H} NMR (125 MHz, dmso-d₆) δ 152.36 (1,2-3′-pzC), 152.09 (2,2-3′-pzC), 151.61 (2,2-2,6-pyC), 151.35 (1,2-6-pyC), 148.55 (1,1-5′-pzC), 148.15 (1,2-5′-pzC), 147.54 (1,2-2-pyC), 147.25 (1,1-2,6-pyC), 142.29 (1,2-5′-pzC), 141.71 (1,1-3′-pzC), 139.42 (2,2-5′-pzC), 138.59 (1,1-4-pyC), 137.58 (1,2-4-pyC), 136.97 (2,2-4-pyC), 117.98 (1,1-3,5-pyCH), 117.77 (1,2-3-pyCH), 117.48 (1,2-5-pyCH), 117.25 (2,2-3,5-pyCH), 103.26 (1,2-4′-pzCH), 103.11 (1,2-4′-pzCH), 102.96 (1,1-4′-pzCH), 102.46 (2,2-4′-pzCH), 56.05 (CH₃CH₂OH), 18.56 (CH₃CH₂OH), 13.56 (1,1 and 1,2-Me), 10.49 (2,2 and 1,2-Me). ¹⁵N NMR (d₆-dmso, indirect detection HMBC, 6 relative to NH₃): 6 304.8 (1,1-pzNH), 302.3 (2,2-pzNH), 289.2 (1,1-pyN), 213.9 (2,2-pzN), 204.2 (1,1-pzN); pyN due to the 2,2-isomer and all N of the 1,2-isomer are not identifiable. After heating at 170° C. under vacuum, the residual de-solvated crystalline powder had a melting point of 207-209° C.

### Example 8b—2,2-(dipyrazolyl)propane [3g]

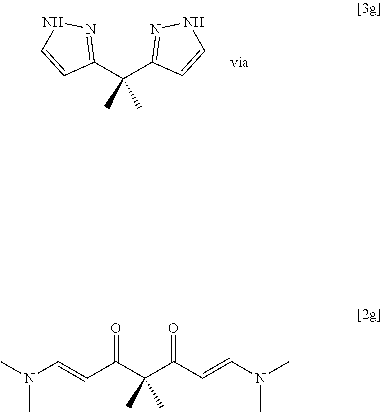

Arm [3g] was prepared as follows:

### 1,7-(bis(N,N-dimethylamino)-4,4-dimethylhepta-1, 6-diene-3,5-dione [2g]

20.3 mL (153 mmol, ca. 4 equiv) of N,N-dimethylformamide dimethyl acetal was added to 5.00 mL (4.89 g, 38.2 mmol) 3,3-dimethyl-2,4-pentanedione and 80 mL xylenes. The reaction vessel was sealed, and the mixture stirred and heated at 130° C. for 48 h, then cooled and the solvent evaporated under vacuum to afford a red-brown oily solid. Washing the solid with hexane afforded a dark brown solid. Yield 3.786 g (15.89 mmol, 42%). ¹H NMR (500 MHz, CDCl₃): δ 7.529 (2H, d, ³J_{HH}=12.5 Hz, N—CH═), 5.010 (2H, d, ³J_{HH}=12.5 Hz, N—CH═CH), 2.999 (6H, br s, NCH₃), 2.726 (6H, br s, NCH₃), 1.274 (6H, s, CMe₂); ¹³C{¹H} NMR (125 MHz, CDCl₃): δ 198.23 (C═O), 152.23 (CH═CHN), 92.63 (CH═CHN), 58.46 (CMe₂), 44.83 (NMe), 37.18 (NMe), 23.04 (CMe₂).

### 2,2-(dipyrazolyl)propane [3g]

2.31 mL (47.7 mmol, ca. 3 equiv) of hydrazine monohydrate was added to 3.786 g (15.89 mmol) of [2g] and 100 mL of anhydrous ethanol in a flask fitted with a reflux condenser and a drying tube. The mixture was refluxed for 24 h to give a deep brown solution. An additional 2.31 mmol (3 equiv) of hydrazine monohydrate was added and reflux continued for another 4 h. On cooling volatiles were removed under reduced pressure to leave a tacky brown semi-solid. This solid was stirred vigorously in 200 mL of pentane overnight. Filtration afforded the product as a beige microcrystalline solid. The solid was dried overnight at 100° C. under vacuum prior to use. Yield: 2.615 g (14.84 mmol, 93%). ¹H NMR (300 MHz, CDCl₃): δ 7.351 (2H, d, ³J_{HH}=2.1 Hz, 5′-pzH), 6.014 (2H, d, ³J_{HH}=2.1 Hz, 4′-pzH), 1.617 (6H, s, CMe₂)*; ¹H NMR (500 MHz, MeOD-d₄): δ 7.460 (2H, br s, 5′-pzH), 6.081 (2H, br s, 4′-pzH), 1.708 (6H, s, CMe₂); ¹³C{¹H} NMR (125 MHz, MeOD-d₄): δ 140.3 (v br s, 3′pzC), 129.6 (v br s, 5′-pzCH), 103.36 (4′-pzCH), 45 (v. br s, CMe₂)**, 29.43 (CMe₂). * NH resonances not observed due to tautomeric exchange; ** tentative.

### Example 9—Tris(2,6-pyrazolylpyridino)diborate dipotassium salt [4a]·i-PrOH·2 H₂O

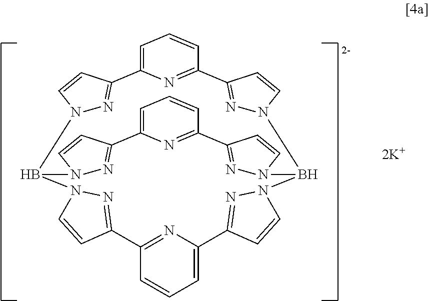

In this example, cage [4a] was prepared in the melt as shown in Scheme 2. Solid [3a](2.23 g, 10.6 mmol, 3 equiv) and KBH₄ (0.382 g, 7.08 mmol, 2 equiv) were ground into a fine powder using a mortar and pestle. The solid mixture was added to a 100 mL round bottom flask equipped. The flask was fitted with a reflux condenser connected by vacuum tubing to an inverted 1 L graduated cylinder, filled with water. The assembled reaction flask was immersed in a silicone oil bath at 200° C. The stirred solids melted as the oil bath temperature was slowly raised to 275° C. Vigorous H₂ gas evolution commenced at around 250° C. and the amount of hydrogen was measured by water displacement (ca. 450 mL, 20 mmol). Once evolution of H₂ ceased, the molten solid re-solidified and no further gas evolution was observed by the time the temperature reached 275° C. The reaction mixture was cooled to room temperature and extracted with 250 mL hot isopropanol. Hot filtration removed some insoluble solid. On cooling the filtrate, pure cage [4a] precipitated as a white powder and was isolated by vacuum filtration. The filtrate contained a mixture of some unreacted [3a] that can be removed by sublimation, as well as unidentified soluble borates. Cage [4a] was isolated as a discrete solvate containing 1 equiv of isopropanol and 2 equiv of H₂O by NMR integration. This matches the TGA analysis below; the best fit to the elemental analysis is for 0.5 equiv of i-PrOH and 2 equiv of water indicating partial desolvation by i-PrOH has occurred under vacuum, consistent with the easy loss of i-PrOH on heating by TGA. Yield of [4a]·i-PrOH·2 H₂O: 1.57 g (56%). Mp.>300° C. ¹H NMR (500 MHz, acetone-d₆): δ 7.82 (6H, d, ³J_{HH}=1.5 Hz, 5′-pzH), 7.68 (3H, t, ³J_{HH}=7.5 Hz, 4-pyH), 7.51 (6H, d, ³J_{HH}=7.5 Hz, 3,5-pyH), 6.68 (6H, d, ³J_{HH}=1.5 Hz, 4′-pzH), 4.93 (2H, br ~q, ¹J_{HB}=96 Hz, BH); ¹³C{¹H} NMR (125 MHz, acetone-d₆): δ 153.89 (2,6-pyC), 153.07 (3′-pzC), 138.34 (4-pyCH), 137.69 (5′-pzCH), 118.72 (3,5-pyCH), 102.88 (4′-pzCHl); ¹¹B NMR (115.59 MHz, d₆-acetone): δ −1.3 (d, ¹J_{BH}=96 Hz); ¹⁵N NMR (d₆-acetone, indirect detection HMBC, δ relative to NH₃): δ 307.9 (2′-pzN), 283.9 (1-pyN), 240.3 (1′-pzN-B). ATR-IR (solid, air background): 2415 cm⁻¹ (w, B—H stretch). High Resolution ESI-MS, neg. mode: [C₃₃H₂₃N₁₅B₂K]⁻, m/z observed: 690.2094; calcd. 690.2084 amu. Elem. analysis calcd. for [4a]·0.5 i-PrOH·2 H₂O: C, 52.09; H, 3.93; N, 26.41%. Found: C, 52.83; H, 3.62; N, 26.43%. TGA: 20-175° C., loss of i-PrOH; 300-350° C., loss of 2 H₂O; >450° C., onset of decomposition. An X-ray crystal structure of K₂ cage [4a]·acetone·2H₂O is shown in FIG. 1.

### Example 10—Tris(2,6-pyrazolyl-4-t-butylpyridino)diborate dipotassium salt [4aa]

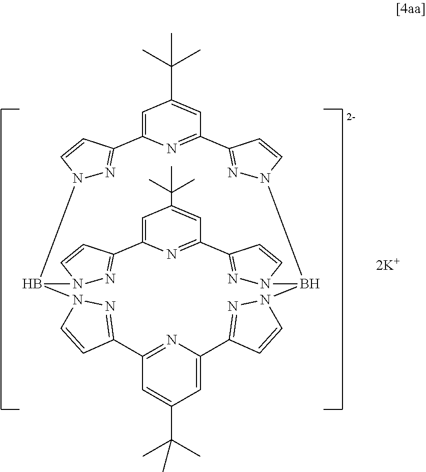

Cage [4aa] was prepared in the melt (Scheme 2) as follows: solid [3aa] (0.150 g, 0.561 mmol, 3 equiv) and KBH₄ (0.020 g, 0.37 mmol, 2 equiv) were ground together with a mortar and pestle and loaded into a small test tube. The test tube was immersed in a silicone oil bath and rapidly heated to 290° C. The solids melted at about 270° C. with notable bubbling. After 15 minutes at 290° C., the sample was removed from the bath and cooled to room temperature. The solid was dissolved in acetone, filtered and the filtrate taken to dryness under reduced pressure. The crude product contained trace KBH₄. Yield: 0.105 g (70%). Mp.>300° C. ¹H NMR (500 MHz, acetone-d₆): δ 7.84 (6H, d, ³J_{HH}=2.5 Hz, 5′-pzH), 7.61 (6H, s, 3,5-pyH), 6.79 (6H, d, ³J_{HH}=2.5 Hz, 4′-pzH), 4.88 (2H, br s, BH), 1.36 (27H, s, CMe₃); ¹³C{¹H} NMR (125 MHz, acetone-d₆): δ 162.19 (4-pyC), 153.45 (2,6-pyC), 153.21 (3′-pzC), 138.34 (4-pyCH), 137.69 (5′-pzCH), 115.67 (3,5-pyCH), 102.60 (4′-pzCH); ¹¹B NMR (160.51 MHz, acetone-d₆): δ 1.1 (v. br s).

### Example 11—Tris(2,6-pyrazolyl-4-trifluoromethylpyridino)diborate dipotassium salt (4f)·acetone

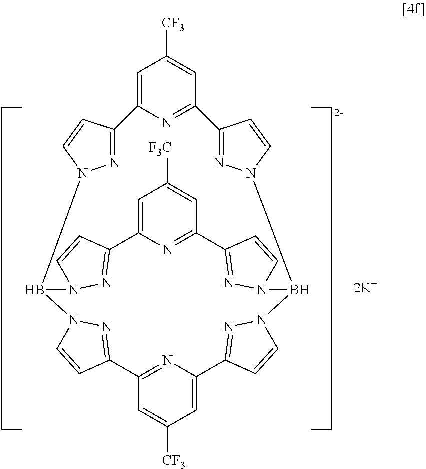

Cage [4f] was prepared in the melt (Scheme 2) as follows: solid [3f] (3.00 g, 10.7 mmol, 3 equiv) and KBH₄ (0.393 g, 7.16 mmol, 2 equiv) were used and the product was washed with hot isopropanol, re-dissolved in acetone, filtered through Celite on a glass frit and the solvent removed under vacuum to afford the acetone solvate. Yield: 1.35 g (38%). Mp.>300° C. ¹H NMR (300 MHz, acetone-d₆): δ 7.86 (6H, d, ³J_{HH}=2.1 Hz, 5′-pzH), 7.76 (6H, s, 3,5-pyH), 6.86 (6H, d, ³J_{HH}=2.1 Hz, 4′-pzH), 4.98 (2H, br ~d, BH), 2.05 (6H, s, (CH₃)₂C═O). ¹³C{¹H} NMR (75 MHz, acetone-d₆): δ 206.26 ((CH₃)₂C═O), 155.67 (2,6-pyC), 151.87 (3′-pzC), 139.45 (q, ²J_{CF}=33 Hz, 4-pyC), 137.19 (5′-pzCH), 124.48 (q, ¹J_{CF}=270 Hz, CF₃), 113.43 (q, ³J_{CF}=3.8 Hz, 3,5-py(CH), 103.44 (4′-pzCH), 29.84 ((CH₃)₂C═O). ¹¹B NMR (115.59 MHz, acetone-d₆): δ −1.4 (d, ¹J_{BH}=84 Hz); ¹⁹F NMR (282.5 MHz, dmso-d₆): δ −63.16. Elem. analysis calcd. for [4f]·acetone: C, 47.24; H, 2.64; N, 21.19%. Found: C, 46.78; H, 2.54; N, 21.49%.

### Example 12—Tris(2,6-pyrazolylpyridino)diborate dipotassium salt [4a]

In this example, cage [4a] was prepared in diglyme solution as shown in Scheme 3. In a typical preparation, dry arm [3a] (3.00 g, 14.2 mmol), KBH₄ (0.511 g, 9.47 mmol) and ca. 10 mol % Y(OTf)₃ (0.500 g, 0.93 mmol) were weighed into a large Kontes flask and dry diglyme (100 mL) was added. The flask was sealed, removed from the glove box and immersed in a silicone oil bath with vigorous stirring. The oil bath was heated at 162° C. for 48 h. On cooling, the flask was opened to air and the contents transferred to a 250 mL round bottom flask. The diglyme solvent was removed by distillation at 70° C. under partial vacuum to leave a tacky white residue. The solid was extracted with 150 mL acetone, filtered through Celite and the solvent removed under reduced pressure. The residue was suspended in 200 mL diethyl ether and stirred for two hours, then filtered to afford cage [4a] as a fine white powder. The product thus obtained is a mono(diglyme) solvate but heating this solvate under vacuum at 120° C. overnight afforded the solvent free cage [4a]. The spectroscopy of this product was essentially identical to that isolated by the melt reaction (without solvation). Yield: 2.35 g (68%).

### Example 13—Tris(2,6-pyrazolyl-4-chloropyridino)diborate dipotassium salt [4b]·diglyme·isopropanol

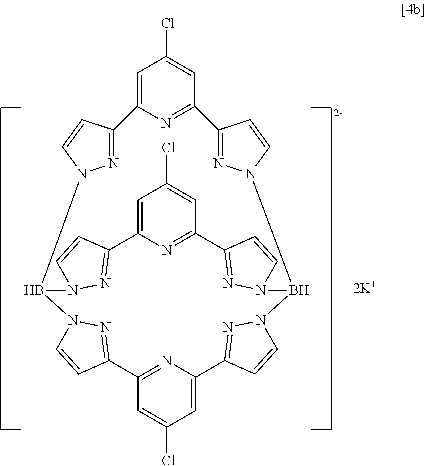

The 4-chloropyridine arm does not melt under 300° C., so the solution reaction shown in Scheme 3 in diglyme solvent was used. The reaction on 0.785 g scale of [3b]afforded approximately 580 mg of cage [4b]after treatment with isopropanol to remove unreacted arm. The cage isolated from this treatment was the mono(diglyme), mono(isopropanol) solvate. Yield: ~53%. Mp.>300° C. ¹H NMR (500 MHz, acetone-d₆): δ 7.794 (6H, d, ³J_{HH}=2.0 Hz, 5′-pzH), 7.534 (6H, s, 3,5-pyH), 6.735 (6H, d, ³J_{HH}=2.0 Hz, 4′-pzH), 4.91 (2H, br m, BH), 3.903 (1H, septet, (CH₃)₂CHOH), 3.537 (4H, t, diglyme CH₂O), 3.456 (4H, t, diglyme CH₂O), 3.276 (6H, s, diglyme OCH₃), 1.109 (6H, d, (CH₃)₂CHOH); ¹³C{¹H} NMR (125 MHz, acetone-d₆): δ 155.78 (2,6-pyC), 151.84 (3′-pzC), 144.86 (4-pyCCl), 137.24 (5′-pzCH), 118.09 (3,5-pyCH), 103.23 (4′-pzCH), 72.61 (diglyme CH₂O), 71.00 (diglyme CH₂O), 63.73 ((CH₃)₂CHOH), 58.76 (diglyme OCH₃), 25.78 ((CH₃)₂CHOH); ¹¹B NMR (160.51 MHz, d₆-acetone): δ −1.37 (br d, ¹J_{BH}=72 Hz); ESI-MS, neg. mode: [C₃₃H₂₀Cl₃N₁₅B₂K]⁻, m/z observed: 794 amu.

### Example 14—Tris(2,6-pyrazolyl-4-bromopyridino)diborate dipotassium salt [4c]

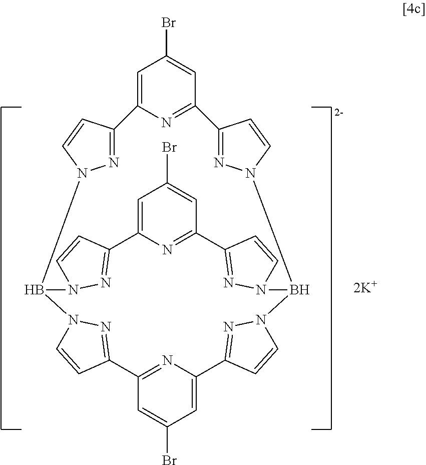

The 4-bromopyridine arm [3c] does not melt under 300° C. so the solution reaction shown in Scheme 3 (diglyme solvent) was used. Using dried 4-bromopyridine arm [3c] on a 2.600 g scale afforded the crude cage as the diglyme adduct after extraction into acetone. Washing the solid with hot isopropanol afforded pure cage free from significant residual arm as an isopropanol solvate. Heating this solvate under vacuum overnight afforded the desolvated cage. Yield after desolvation: 1.997 g (69%). Mp.>300° C. ¹H{¹¹B} NMR (500 MHz, acetone-d₆): δ 7.803 (6H, d, ³J_{HH}=2.5 Hz, 5′-pzH), 7.700 (6H, s, 3,5-pyH), 6.748 (6H, d, ³J_{HH}=2.5 Hz, 4′-pzH), 4.91 (2H, br m, BH); ¹³C{¹H} NMR (125 MHz, acetone-d₆): δ 155.51 (2,6-pyC), 151.67 (3′-pzC), 137.37 (5′-pzCHl), 133.72 (4-pyC-Br), 121.11 (3,5-pyCH), 103.29 (4′-pzCHl); ¹¹B NMR (115.59 MHz, acetone-d₆): δ −1.36 (br d, ¹J_{BH}=67 Hz); ESI-MS, neg. mode: [C₃₃H₂₀Br₃N₁₅B₂K]⁻, m/z observed: 928 amu. An X-ray crystal structure of Ba (cage [4c]), crystallized from diethyl ether, is shown in FIG. 2. The structure is a centrosymmetric dimer that is bridged through one set of 4-Br-pyridine arms on each cage. The barium is therefore likely 10-coordinate, rather than 9-coordinate, and sits approximately in the center of the cage.

### Example 15—Tris(2,6-pyrazolyl-4-iodopyridino)diborate dipotassium salt [4d]·1.5 diglyme solvate

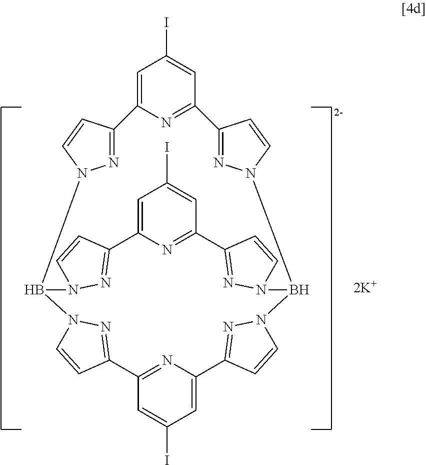

The 4-iodopyridine arm [3d]decomposes during melt reactions so the solution reaction shown in Scheme 3 in diglyme solvent was used. The reaction on 1.00 g scale of [3d] afforded ca. 0.895 g of cage. Yield: 73%. Mp.>300° C. ¹H NMR (500 MHz, 10:1 acetone-d₆: dmso-d₆): δ 7.904 (6H, s, 3,5-pyH), 7.756 (6H, d, ³J_{HH}=2.2 Hz, 5′-pzH), 6.731 (6H, d, ³J_{HH}=2.2 Hz, 4′-pzH), 4.82 (2H, v. br −d, BH), 3.555 (6H, t, diglyme, CH₂OCH₂), 3.463 (6H, t, diglyme, CH₂OCH₃), 3.282 (9H, s, diglyme, OCH₃); ¹³C{¹H} NMR (125 MHz, 10:1 acetone-d₆: dmso-d₆): δ 154.55 (2,6-pyC), 150.96 (3′-pzC), 136.77 (5′-pzCH), 126.90 (3,5-pyCH), 106.55 (4-pyC-I), 103.04 (4′-pzCH), 72.69 (CH₂OCH₃), 71.08 (CH₂OCH₂), 58.85 (OCH₃); ¹¹B NMR (160.51 MHz, 10:1 acetone-d₆: dmso-d₆): δ −1.5 (v. br s, BH coupling not resolved); ¹⁵N NMR (dmso-d₆, indirect detection HMBC, δ relative to NH₃): δ 316.8 (2′-pzN), 208.4 (1′-pzN-B); pyN not observed, assignments tentative as the signal is very weak. ESI-MS, neg. mode: [C₃₃H₂₀I₃N₁₅B₂K]⁻, m/z observed: 1068 amu.

### Example 16—Tris(2,6-pyrazolyl-4-cyanopyridino)diborate dipotassium salt [4e]·0.5 triglyme

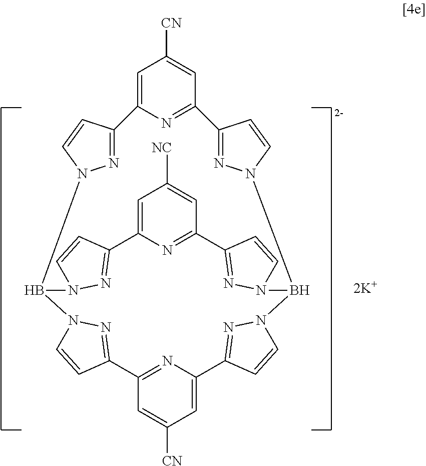

Although the 4-cyanopyridine arm [3e] does melt, substantial decomposition occurs so the melt reaction shown in Scheme 2 produces low yields of impure product. However, carrying out the reaction on 2.65 g arm scale in triglyme according to Scheme 3 affords [4e] in good yield. Extraction with acetone, filtration to remove bright yellow impurities and removal of the solvent left crude cage [4e]contaminated by some arm [3e]. Washing the solid with a small amount of hot isopropanol afforded [4e]free from significant residual arm. The color of the product varied from off-white to pale orange with no evident change in spectroscopy. Yield: 2.31 g, 60%. Mp.>300° C. ¹H{¹¹B} NMR (500 MHz, acetone-d₆): δ 7.816 (6H, d, ³J_{HH}=2.2 Hz, 5′-pzH), 7.806 (6H, s, 3,5-pyH), 6.816 (6H, d, ³J_{HH}=2.2 Hz, 4′-pzH), 4.93 (2H, s, BH), 3.541 (4H, t, CH₃OCH₂CH₂O), 3.537 (4H, s, OCH₂CH₂O), 3.456 (4H, t, CH₃OCH₂CH₂O), 3.271 (6H, s, OCH₃); ¹³C{¹H} NMR (125 MHz, acetone-d₆): δ 155.45 (2,6-pyC), 151.47 (3′-pzC), 137.23 (5′-pzCH), 121.75 (CN), 119.61 (3,5-pyCH), 118.07 (4-pyC-CN), 103.61 (4′-pzCH), 72.47 (CH₃OCH₂), 70.94 (CH₃OCH₃CH₂O), 70.78 (OCH₂CH₂O), 58.77 (OCH₃); ¹¹B NMR (160.5 MHz, acetone-d₆): δ −1.43 (br d, ¹J_{BH}=74 Hz); ESI-MS, neg. mode: [C₃₆H₂₀N₁₈B₂K]⁻, m/z observed: 765 amu.

### Example 17—Tris(2,6-pyrazolyl-4-pyridinecarboxylic acid, lithium salt)diborate dipotassium salt [4g]

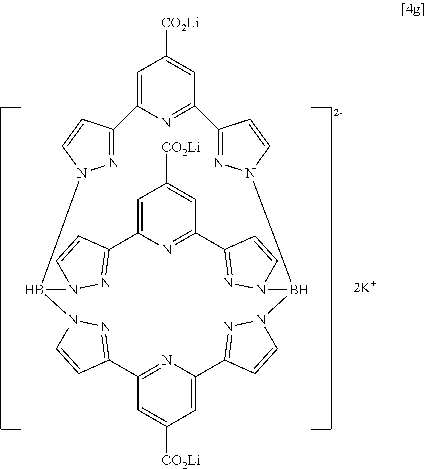

Dried and de-solvated (160° C. under vacuum overnight) bromo cage [4c](0.143 g, 0.153 mmol) was dissolved in 10 mL of dry THE in a small Schlenk flask in the glovebox. After removal to the vacuum line, the solution was cooled to −78° C. under nitrogen and 2.5 M n-BuLi solution (0.183 mL, 0.46 mmol) was added by syringe resulting in an immediate color change to deep red-brown. After stirring for 1 minute, CO₂ gas (dried by passing through a Drierite cartridge) was bubbled through the solution resulting in a color change to yellow and finally pale grey. Removal of the solvent under reduced pressure and washing with diethyl ether afforded cage [4g] as an off-white solid. Yield: 0.085 g (66%). Mp. dec.>310° C. ¹H{¹¹B} NMR (500 MHz, D₂O): δ 8.001 (6H, d, ³J_{HH}=2.5 Hz, 5′-pzH), 7.925 (6H, s, 3,5-pyH), 6.857 (6H, d, ³J_{HH}=2.5 Hz, 4′-pzH), 4.95 (2H, br s, BH); ¹³C{¹H} NMR (125 MHz, D₂O): δ 174.38 (CO₂⁻Li⁺), 153.73 (2,6-pyC), 153.43 (3′-pzC), 147.00 (4-pyC-CO₂), 138.88 (5′-pzCH), 119.34 (3,5-pyCH), 104.49 (4′-pzCH); ¹¹B NMR (115.59 MHz, D₂O): δ −2.34 (br s).

### Example 18—Tris(2,6-pyrazolyl-4-acetyl pyridine)diborate dipotassium salt [4h], 0.5 triglyme solvate

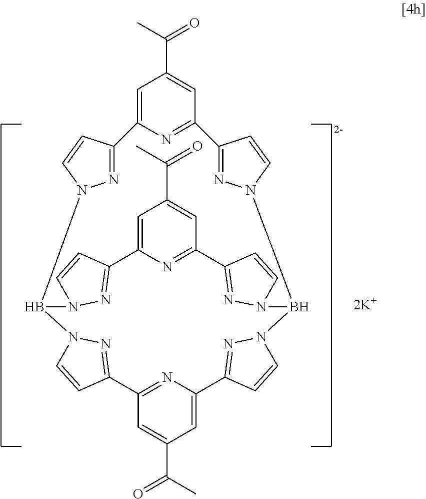

Dried cyano cage [4e] (0.157 g, 0.195 mmol) was weighed out into a Kontes flask and dissolved in 8 mL dry THE in the glovebox. The flask was sealed and removed to the vacuum line and cooled with an ice bath. A solution of methyl magnesium bromide (0.46 mL, 1.4 M, 0.64 mmol, 3.3 mmol) was added. After stirring for 30 minutes at room temperature, the sealed flask was heated at 65° C. in an oil bath overnight. After cooling, the flask was opened and the black solution was quenched with 2 mL of water. The resulting yellow-orange solution was stirred for 30 minutes and the solvent removed under vacuum. The solid residue was re-dissolved in acetone, filtered through Celite and the filtrate was taken to dryness. The residue was washed with diethyl ether and filtered to give a yellow solid. Yield: 0.155 g (93%). Mp. dec.>310° C. ¹H{¹¹B} NMR (500 MHz, dmso-d₆): δ 7.903 (6H, s, 3,5-pyH), 7.815 (6H, d, ³J_{HH}=2.5 Hz, 5′-pzH), 6.894 (6H, d, ³J_{HH}=2.5 Hz, 4′-pzH), 4.80 (2H, br s, BH), 3.541 (2H, t, CH₃OCH₂CH₂O), 3.537 (2H, s, OCH₂CH₂O), 3.456 (2H, t, CH₃OCH₂CH₂O), 3.271 (3H, s, OCH₃), 2.684 (9H, s, CH₃); ¹³C{¹H} NMR (125 MHz, dmso-d₆): δ 198.58 (C═O), 153.70 (2,6-pyC), 151.00 (3′-pzC), 144.51 (4-pyC-C═O), 136.49 (5′-pzCH), 115.33 (3,5-pyCH), 102.87 (4′-pzCH), 71.28 (CH₃OCH₂), 69.80 (CH₃OCH₃CH₂O), 69.59 (OCH₂CH₂O), 58.06 (OCH₃), 27.18 (CH₃); ¹¹B NMR (115.59 MHz, dmso-d₆): δ −2.46 (v. br s).

### Example 19—Tris(2,6-pyrazolyl-4-(carboxamido)pyridine)diborate dipotassium salt, [4i]

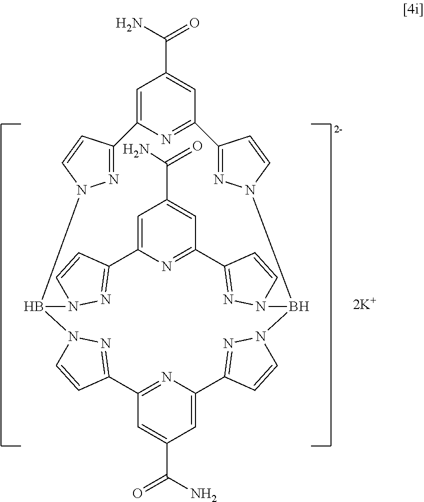

Dried cyano cage [4e] (0.120 g, 0.149 mmol) and potassium t-butoxide (0.155 g, 1.37 mmol, 9 equiv) were weighed into a Kontes flask in the glovebox and 5 mL of dry THE was added resulting in an intense purple solution. The flask was sealed, removed to a vacuum line and placed under nitrogen. t-Butanol (5 mL) was added, the flask re-sealed and the deep purple solution was stirred for 48 h at room temperature. After 48 h, the flask was opened, and ca. 5 mL of water was added followed by vigorous stirring open to air. As the solution stirred, the purple color faded to yellow-orange after 30 min. Removal of the solvent left a greenish-brown residue that re-dissolved in dmso-d₆ to give a red-purple solution, but shaking the sample in air again caused a color change to yellow-orange. Over time, the yellow-orange NMR sample darkened to red-purple but each time shaking returned the color to yellow-orange, possibly suggesting the presence of radical species. ¹H{¹¹B} NMR (500 MHz, dmso-d₆): δ 7.998 (6H, s, 3,5-pyH), 7.761 (6H, d, ³J_{HH}=2.5 Hz, 5′-pzH), 7.707 (3H, s, NH, tentative, overlaps previous peak), 6.720 (3H, br s, NH, tentative, overlaps next peak), 6.678 (6H, d, ³J_{HH}=2.5 Hz, 4′-pzH), 4.79 (2H, br s, BH), 3.720 (2H, t, CH₃OCH₂CH₂O), 3.506 (2H, s, OCH₂CH₂O), 3.436 (2H, br s, CH₃OCH₂CH₂O), 3.246 (3H, s, OCH₃); ¹³C{¹H} NMR (125 MHz, dmso-d₆): δ 170.04 (C═O), 152.04 (2,6-pyC), 151.75 (3′-pzC), 135.86 (4-pyC-C═O), 135.69 (5′-pzCH), 117.05 (3,5-pyCH), 101.76 (4′-pzCH), 71.22 (CH₃OCH₂), 69.72 (CH₃OCH₃CH₂O), 69.51 (OCH₂CH₂O), 58.04 (OCH₃); ¹¹B NMR (115.59 MHz, dmso-d₆): δ −2.42 (br s).

### Example 20—Tris(2,6-pyrazolyl-4-(amidrazonyl)pyridine)diborate dipotassium salt, [4j], mono(triglyme) solvate

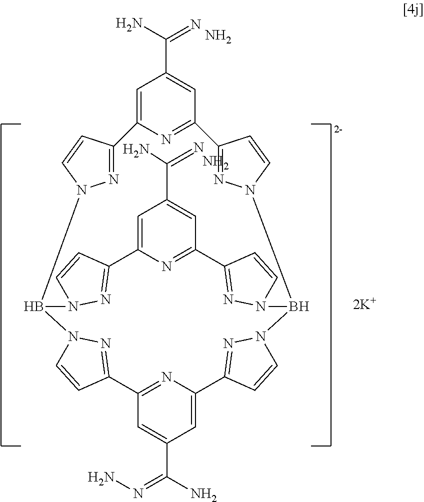

Dried cyano cage [4e] (0.150 g, 0.186 mmol) was dissolved in 8 mL of hydrazine monohydrate in a small round bottom flask in air. The flask was sealed, covered with aluminum foil and stirred vigorously at room temperature overnight. The solvent was removed under vacuum and the solid residue washed with diethyl ether by stirring for several hours. The solid yellow product was isolated by filtration and dried under vacuum to yield [4j]0.138 g (83%). Mp. dec.>315° C. ¹H{¹¹B} NMR (500 MHz, dmso-d₆): S 7.813 (6H, s, 3,5-pyH), 7.776 (6H, d, ³J_{HH}=2.5 Hz, 5′-pzH), 6.710 (6H, d, ³J_{HH}=2.5 Hz, 4′-pzH), 5.82 (6H, br s, NH₂), 4.79 (2H, br s, BH), 3.541 (4H, t, CH₃OCH₂CH₂O), 3.537 (4H, s, OCH₂CH₂O), 3.46 (6H, v. br s, NH₂), 3.456 (4H, t, CH₃OCH₂CH₂O), 3.271 (6H, s, OCH₃); ¹³C{¹H} NMR (125 MHz, dmso-d₆): δ 152.48 (2,6-pyC), 151.56 (3′-pzC), 143.41 (C(NH₂)═NNH₂), 142.55 (4-pyC-C(NH₂)═N), 136.00 (5′-pzCH), 113.37 (3,5-pyCH), 102.05 (4′-pzCH), 71.28 (CH₃OCH₂), 69.80 (CH₃OCH₃CH₂O), 69.59 (OCH₂CH₂O), 58.06 (OCH₃); ¹¹B NMR (115.59 MHz, dmso-d₆): δ −2.31 (v. br s). ESI-MS, neg. mode: [M+K⁺]⁻, [C₃₆H₃₂N₂₄B₂K]⁻: m/z observed: 861 amu.

### Example 21—Tris(2,6-pyrazolyl-4-(1′-oxahept-6′-enyl)pyridino)diborate dipotassium salt

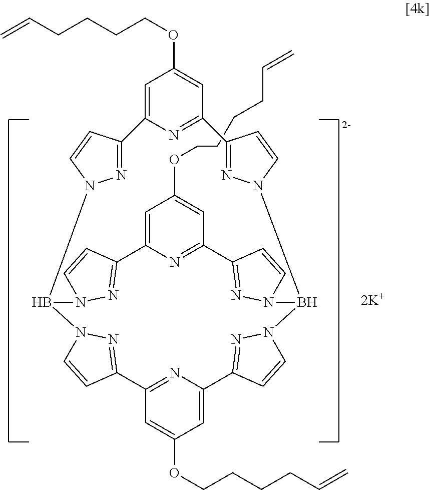

In a glove box, a Kontes flask was charged with 0.025 g (1.09 mmol, 6 equiv) sodium in 4 mL dry THF. The flask was removed from the glove box, and 5-hexen-1-ol (0.13 mL, 1.09 mmol, 6 equiv) was added under nitrogen. The flask was sealed and, the suspension heated with stirring at 80° C. overnight to allow all the sodium to react. After cooling, a solution of the potassium salt of Br-cage [4c] (0.200 g, 0.18 mmol, 1 equiv) in 5 mL dry THF was added to the cloudy orange suspension of sodium 5-hexen-1-alkoxide. The flask was resealed, and the suspension heated at 80° C. for 48 h. After cooling and removal of the solvent under reduced pressure, the flask was opened and 50 mL of distilled water was added. The resulting suspension was stirred 4 h, filtered to isolate the solid and the residue washed with additional distilled water. Drying of the solid under vacuum afforded the product [4k] as a pale off-white solid. Yield: 0.131 g (73%). Mp.>300° C. ¹H{¹¹B} NMR (500 MHz, acetone-d₆): δ 7.754 (6H, d, ³J_{HH}=2.8 Hz, 5′-pzH), 7.062 (6H, s, 3,5-pyH), 6.652 (6H, d, ³J_{HH}=2.8 Hz, 4′-pzH), 5.83 (3H, m, 6′-CH═CH₂), 5.014 (3H, d, ³J_{HH}=17.5 Hz, trans-7′-CH═CHₜᵣₐₙₛH_{cis}), 4.926 (3H, d, ³J_{HH}=10.0 Hz, cis-7′-CH═CHₜᵣₐₙₛH_{cis}), 4.89 (2H, br m, BH), 4.133 (6H, t, ³J_{HH}=6.3 Hz, 2′-OCH₂), 2.119 (6H, q, ³J_{HH}=7.5 Hz, 5′-CH₂CH═), 1.795 (6H, pentet, ³J_{HH}=7.0 Hz, 3′-OCH₂CH₂), 1.565 (6H, pentet, ³J_{HH}=7.6 Hz, 4′-CH₂CH₂CH═); ¹³C{¹H} NMR (125 MHz, acetone-d₆): δ 167.22 (4-pyC-O), 155.70 (2,6-pyC), 153.07 (3′-pzC), 139.44 (6′-CH═), 136.90 (5′-pzCHl), 105.12 (3,5-pyCH), 103.05 (7′-CH═CH₂), 102.30 (4′-pzCHl), 68.29 (2′-OCH₂), 34.07 (5′-CH₂CH═), 29.27 (3′-OCH₂CH₂), 25.99 (4′-CH₂CH₂CH═); ¹¹B NMR (115.59 MHz, acetone-d₆): δ −1.31 (br s); ESI-MS, pos. mode: [M+3H⁺]⁺, [C₅₁H₅₆O₃N₁₅B₂]⁺: m/z observed: 948 amu; ESI-MS, neg. mode: [M+K⁺]⁻, [C₅₁H₅₃O₃N₁₅B₂K]⁻: m/z observed: 984 amu.

### Example 22—Tris(2,6-pyrazolyl-4-(1′,4′,7′-trioxaoctanyl)pyridino)diborate dipotassium salt [41]

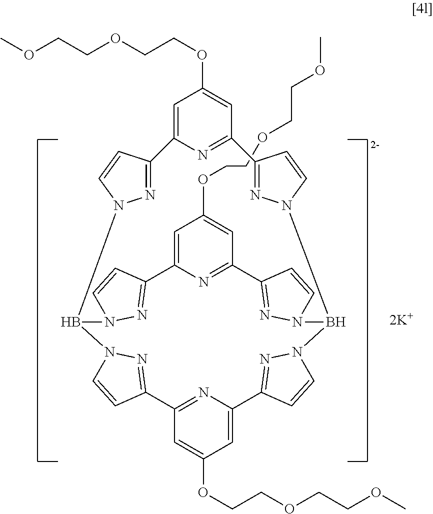

A Kontes flask was charged with 0.025 g (1.09 mmol, 6 equiv) sodium in 3 mL dry THF. 2-(2′-methoxyethoxy)ethanol (0.13 mL, 1.09 mmol, 6 equiv) was added under nitrogen. The flask was sealed, and the suspension heated with stirring at 80° C. overnight to allow all the sodium to react. After cooling, a solution of the potassium salt of Br-cage [4c] (0.200 g, 0.18 mmol, 1 equiv) in 5 mL dry THE was added to the cloudy red suspension of sodium 2-(2-methoxyethoxy)ethoxide. The flask was resealed, and the suspension heated at 80° C. for 48 h. After cooling, the dark red solution was evaporated to dryness under reduced pressure. The flask was opened and 50 mL of distilled water was added. The resulting suspension was stirred 24 h, filtered to isolate the solid and the residue washed with additional distilled water. Drying of the solid under vacuum afforded the product as a pale off-white solid. Yield: 0.177 g (95%). Mp. 172-175° C. ¹H{¹¹B} NMR (500 MHz, dmso-d₆): δ 7.745 (6H, br s, 5′-pzH), 7.114 (6H, br s, 3,5-pyH), 6.750 (6H, br s, 4′-pzH), 4.75 (2H, br s, BH), 4.249 (6H, br s, 2′-CH₂), 3.726 (6H, br s, 3′-CH₂), 3.554 (6H, br s, 5′-CH₂), 3.421 (6H, br s, 6′-CH₂), 3.198 (9H, br s, 8′-OCH₃); ¹³C{¹H} NMR (125 MHz, dmso-d₆): δ 167.02 (4-pyC-O), 154.43 (2,6-pyC), 151.75 (3′-pzC), 136.42 (5′-pzCH), 104.67 (3,5-pyCH), 102.70 (4′-pzCH), 71.56 (6′-CH₂), 70.03 (5′-CH₂), 69.05 (3′-CH₂), 67.58 (2′-CH₂), 58.40 (8′-OCH₃). ¹¹B NMR (115.59 MHz, acetone-d₆): δ −1.5 (v. br s); ESI-MS, neg. mode: [M+K⁺]⁻, [C₄₈H₅₃O₉N₁₅B₂K]⁻: m/z observed: 1044 amu.

### Example 23—Tris(2,6-pyrazolyl-4-(1′,4′,7′,10′,13′-pentaoxatetradecanyl)pyridino)diborate dipotassium salt [4m]

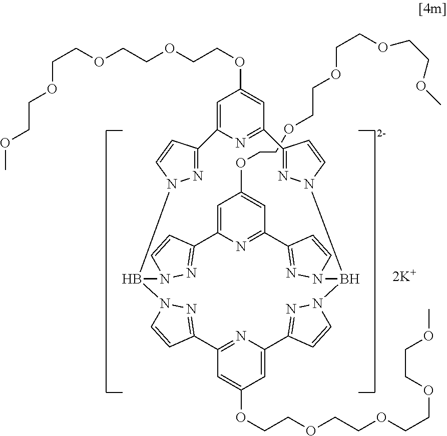

Compound [4m] was prepared by the same route as [41] using tetraethylene glycol monomethyl ether (0.260 g, 1.24 mmol, 6 equiv) and Br-cage [4c] (0.200 g, 0.207 mmol, 1 equiv) in 5 mL dry THF. Yield: 0.070 g (25%). Mp.>300° C. ¹H{¹¹B} NMR (500 MHz, acetone-d₆:dmso-d₆ 10:1): δ 7.725 (6H, br d, ³J_{HH}=2.5 Hz, 5′-pzH), 7.083 (6H, br s, 3,5-pyH), 6.666 (6H, br d, ³J_{HH}=2.5 Hz, 4′-pzH), 4.86 (2H, br s, BH), 4.265 (6H, br t, 2′-CH₂), 3.813 (6H, br t, 3′-CH₂), 3.628 (6H, br t, 5′-CH₂), 3.56 (12H, br m, 6′ and 8′-CH₂), 3.53 (12H, br m, 9′ and 11′-CH₂), 3.419 (6H, br t, 12′-CH₂), 3.230 (9H, br s, 14′-OCH₃); ¹³C{¹H} NMR (125 MHz, acetone-d₆:dmso-d₆ 10:1): δ 167.57 (4-pyC-O), 155.60 (2,6-pyC), 152.59 (3′-pzC), 136.20 (5′-pzCH), 104.92 (3,5-pyCH), 102.11 (4′-pzCH), 72.31, 71.06, 70.89, 70.82, 70.66, 70.60, 69.92, 67.90 (2′,3′,5′,6′,8′,9′,11′,12′-CH₂), 58.64 (14′-OCH₃). ¹¹B NMR (115.59 MHz, acetone-d₆): δ −1.33 (br s).

### Example 24—Tris(2,6-pyrazolyl-4-(1′,4′,7′,10′,13′,16′,19′-heptaoxaeicosanyl)pyridino)diborate dipotassium salt [4n]

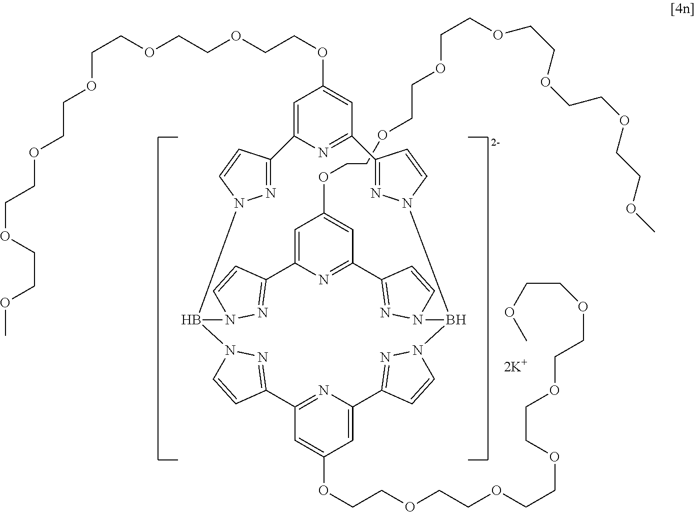

Compound [4n] was prepared by the same route as [41] using hexaethylene glycol monomethyl ether (0.368 g, 1.24 mmol, 6 equiv) and Br-cage [4c] (0.200 g, 0.207 mmol, 1 equiv) in 5 mL dry THF. Yield: 0.155 g (46%). Mp.>300° C. ¹H{¹¹B} NMR (500 MHz, acetone-d₆): δ 7.733 (6H, br d, ³J_{HH}=2.5 Hz, 5′-pzH), 7.025 (6H, br s, 3,5-pyH), 6.609 (6H, br d, ³J_{HH}=2.5 Hz, 4′-pzH), 4.88 (2H, br s, BH), 4.175 (6H, br t, 2′-CH₂), 3.738 (6H, br t, 3′-CH₂), 3.37-3.55 (60H, m, 5′,6′,8′,9′,11′,12′,14′,15′,17′,18′-CH₂), 3.205 (9H, br s, 20′-OCH₃); ¹³C{¹H} NMR (125 MHz, acetone-d₆): δ 166.66 (4-pyC-O), 155.95 (2,6-pyC), 152.78 (3′-pzC), 136.29 (5′-pzCH), 105.22 (3,5-pyCH), 102.83 (4′-pzCH), 72.24, 72.19, 71.01, 70.98, 70.75, 70.72, 70.69, 70.44, 70.03, 70.00, 69.97, 67.87 (2′,3′,5′,6′,8′,9′,11′,12′,14′,15′,17′,18′-CH₂), 58.78 (20′-OCH₃). ¹¹B NMR (115.59 MHz, acetone-d₆): δ −1.33 (br s).

### Example 25—Tris(2,6-pyrazolyl-4-(1′,4′,7′-trioxanon-8′-enyl)pyridino)diborate dipotassium salt [40]

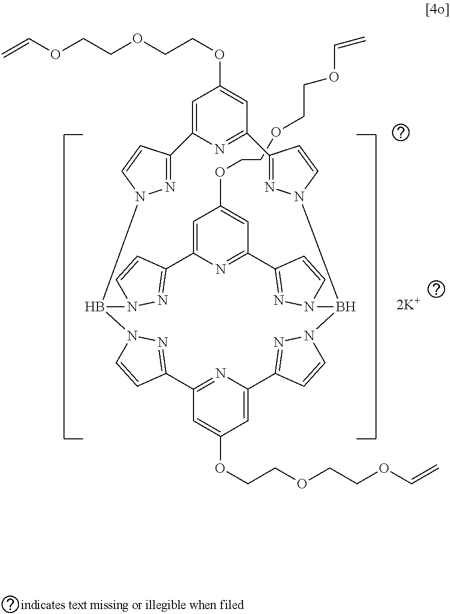

Compound [40] was prepared by the same route as [41] using di(ethylene glycol) vinyl ether (0.165 g, 1.24 mmol) and Br-cage [4c] (0.200 g, 0.207 mmol, 1 equiv) in 5 mL dry THF. Yield: 0.170 g (73%). Mp. 185-188° C. ¹H{¹¹B} NMR (500 MHz, acetone-d₆): δ 7.765 (6H, d, ³J_{HH}=2.0 Hz, 5′-pzH), 7.083 (6H, s, 3,5-pyH), 6.673 (6H, d, ³J_{HH}=2.0 Hz, 4′-pzH), 6.463 (3H, dd, ³J_{HH}=14.3, 6.7 Hz, 8′-CH═CH₂), 4.89 (2H, br s, BH), 4.246 (6H, t, ³J_{HH}=4.8 Hz, 2′-CH₂), 4.154 (3H, dd, ³J_{HH}=14.3 Hz, ²J_{HH}=2.0 Hz, 9′-CH═CHₜᵣₐₙₛH_{cis}), 3.912 (3H, dd, ³J_{HH}=6.7 Hz, ²J_{HH}=2.0 Hz, 9′-CH═CHₜᵣₐₙₛH_{cis}), 3.80-3.83 (12H, m, 3′ and 5′-CH₂), 3.71-3.73 (6H, m, 6′-CH₂); ¹³C{¹H} NMR (125 MHz, acetone-d₆): δ 167.04 (4-pyC-O), 155.72 (2,6-pyC), 153.38 (8′-OCH═), 153.06 (3′-pzC), 136.96 (5′-pzCH), 105.20 (3,5-pyCH), 102.41 (4′-pzCH), 86.37 (9′-CH═CH₂), 70.33, 70.14, 68.36, 68.26 1 (2′, 3′, 5′ and 6′-OCH₂). ¹¹B NMR (115.59 MHz, acetone-d₆): δ −1.40 (br s); ESI-MS, pos. mode: [M+3H⁺]⁺, [C₅₁H₅₆O₉N₁₅B₂]⁺: m/z observed: 1044 amu.

### Example 26—Tris(2,6-pyrazolyl-4-(3′-hydroxypropan-1′-oxy)pyridino)diborate dipotassium salt [4p]

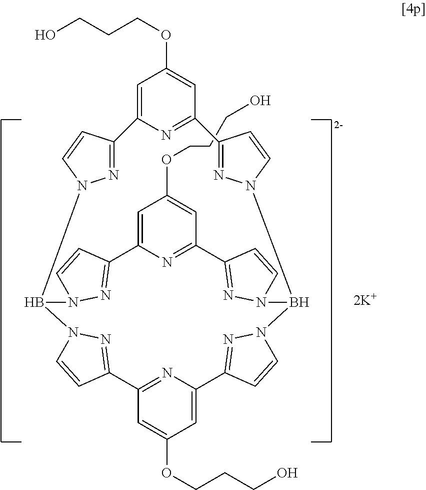

Compound [4p] was prepared by a similar route to compound [41]. A Kontes flask was charged with propanediol (propylene glycol) (0.5 mL, 7.0 mmol, 34 equiv) and sodium metal (0.029 g, 1.2 mmol, 6 equiv) in 3 mL THE under nitrogen and stirred at 90° C. overnight. Br-cage [4c] (0.200 g, 0.207 mmol, 1 equiv) in ca. 2 mL dry THE was added, the flask re-sealed and the orange solution was heated at 90° C. for 48 h. Removal of the solvent under reduced pressure afforded a syrupy suspension that was washed by stirring it in diethyl ether overnight, decanting off the ether, and air drying to afford a white paste. Further treatment with diethyl ether produced a white powder that was filtered off and washing with more ether and vacuum dried to a white powder. Yield: 0.050 g (25%). ¹H{¹¹B} NMR (500 MHz, acetone-d₆): δ 7.750 (6H, d, ³J_{HH}=2.5 Hz, 5′-pzH), 7.063 (6H, s, 3,5-pyH), 6.652 (6H, d, ³J_{HH}=2.5 Hz, 4′-pzH), 4.89 (2H, br s, BH), 4.215 (6H, t, ³J_{HH}=6.3 Hz, 2′-CH₂), 3.912 (6H, m, 4′-CH₂OH), 3.709 (3H, br t, CH₂OH), 1.955 (6H, pentet, ³J_{HH}=6.0 Hz, CH₂CH₂CH₂); ¹³C{¹H} NMR (125 MHz, acetone-d₆): δ 167.20 (4-pyC-O), 155.73 (2,6-pyC), 153.04 (3′-pzC), 136.82 (5′-pzCH), 104.54 (3,5-pyCH), 102.28 (4′-pzCH), 65.47, 58.82 (2′ and 4′-CH₂O), 33.14 (CH₂CH₂CH₂). ¹¹B NMR (115.59 MHz, acetone-d₆): δ −1.27 (br s); ESI-MS, neg. mode: [M+K]⁻, [C₄₂H₄₁O₆N₁₅B₂K]⁻: m/z observed: 912 amu.

### Example 27—Tris(2,6-pyrazolyl-4-(2′-(10″-anthracenyl)methoxy)pyridino)diborate dipotassium salt [4q]

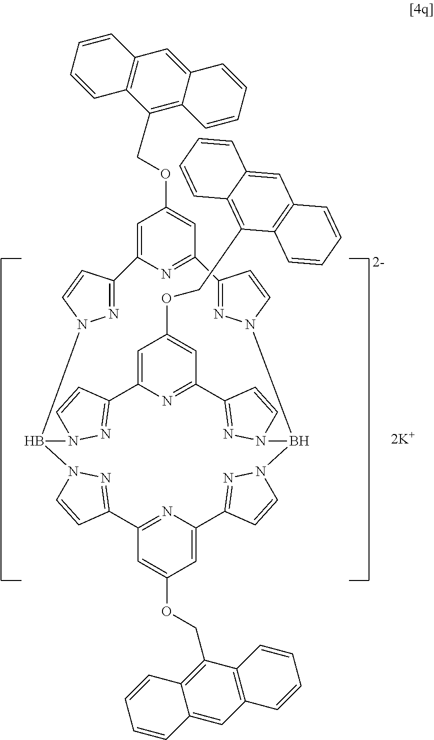

Compound [4q] was prepared by a similar route to compound [41]. A Kontes flask was charged with anthracenyl methanol (0.250 g, 1.2 mmol, ca. 6 equiv) and sodium metal (0.029 g, 1.2 mmol, 6 equiv) in 10 mL THE under nitrogen and stirred at 50° C. overnight to produce an orange solution of the anthracenyl methoxide. Br-cage [4c] (0.200 g, 0.207 mmol, 1 equiv) in ca. 5 mL dry THE was added, the flask re-sealed and the orange solution was heated at 90° C. for 48 h. Addition of water (ca. 1 mL) and removal of the solvent under reduced pressure afforded a tacky orange solid. The product was extracted into acetone (50 mL), filtered through Celite in air and the filtrate taken to dryness to afford a red-brown oil containing excess anthracenyl methanol. To this was added 200 mL of diethyl ether to precipitate the product. The suspension was stirred overnight and the solid isolated as a beige solid. The solid can be purified by extraction into dichloromethane. Yield: 0.178 g (60%). ¹H{¹¹B} NMR (500 MHz, 3:1 acetone-d₆:dmso-d₆): δ 8.71 (3H, s, 5″-anthH), 8.44 (6H, d, ³J_{HH}=7.0 Hz, 1″,9″-anthH), 8.16 (6H, d, ³J_{HH}=7.0 Hz, 4″,6″-anthH), 7.75 (6H, m, 5′-pzH), 7.62 (6H, m, 2″,8″-anthH), 7.55 (6H, m, 3″,7″-anthH), 7.40 (6H, m, 3,5-pyH), 6.75 (6H, m, 4′-pzH), 6.31 (6H, s, CH₂O), 4.88 (2H, br s, BH); ¹³C{¹H} NMR (125 MHz, 3:1 acetone-d₆:dmso-d₆): δ 166.15 (4-pyC-O), 154.76 (2,6-pyC), 154.38 (3′-pzC), 135.62 (5′-pzCH), 131.27, 130.84, 129.00, 126.91, 126.53, 124.24 (anthC), 104.94 (3,5-pyCH), 101.51 (4′-pzCH), 62.29 (CH₂O); ¹¹B NMR (115.59 MHz, 3:1 acetone-d₆:dmso-d₆): δ −1.35 (br s); ESI-MS, neg. mode: [M+K]⁻, [C₇₈H₅₃O₃N₁₅B₂K]⁻: m/z observed: 1308 (very weak M⁺), 1119 (M+−anthr-CH₂OH+H₂O), 928 (M⁺−2 anthr-CH₂OH+MeOH), 912 (M⁺−2 anthr-CH₂OH+H₂O) amu.

### Example 28—Tris(2,6-pyrazolyl-4-(3′-thiolatopropan-1′-thio)pyridino)diborate dipotassium salt [4r]

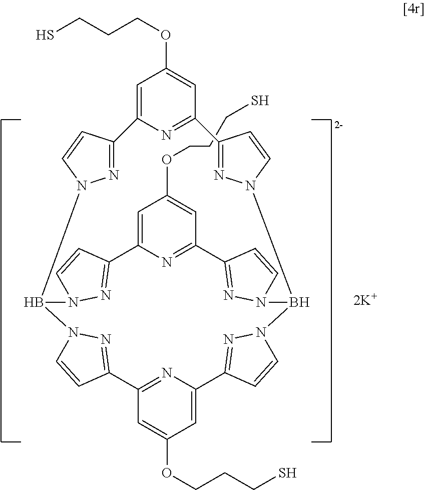

Compound [4r] was prepared by a similar route to compound [4l] except that propanedithiol (0.15 g, 1.4 mmol, ca. 6 equiv) was deprotonated with NaH (0.034 g, 1.4 mmol, ca. 6 equiv) before addition of Br-cage [4c] (0.200 g, 0.207 mmol, 1 equiv) in 5 mL dry THF. Yield: 0.145 g (84%). ¹H{¹¹B} NMR (500 MHz, acetone-d₆): δ 7.744 (6H, d, ³J_{HH}=2.5 Hz, 5′-pzH), 7.396 (6H, s, 3,5-pyH), 6.709 (6H, d, ³J_{HH}=2.5 Hz, 4′-pzH), 4.85 (2H, br s, BH), 3.246 (6H, t, ³J_{HH}=7.5 Hz, 2′-CH₂), 2.660 (6H, q, ³J_{HH}=7.0 Hz, 4′-CH₂SH), 2.253 (6H, pentet, ³J_{HH}=6.5 Hz, 3′-CH₂), 1.964 (3H, t, ³J_{HH}=7.5 Hz, CH₂SH); ¹³C{¹H} NMR (125 MHz, acetone-d₆): δ 155.75 (2,6-pyC), 152.05 (3′-pzC), 148.94 (4-pyC-S), 136.50 (5′-pzCH), 115.28 (3,5-pyCH), 103.14 (4′-pzCH), 39.22 (—SCH₂), 34.00 (CH₂SH), 23.56 (CH₂CH₂CH₂); ¹¹B NMR (115.59 MHz, acetone-d₆): δ −1.42 (br s).

### Example 29—Tris(2,6-pyrazolyl-4-(benzylthiolato)pyridino)diborate dipotassium salt [4s]

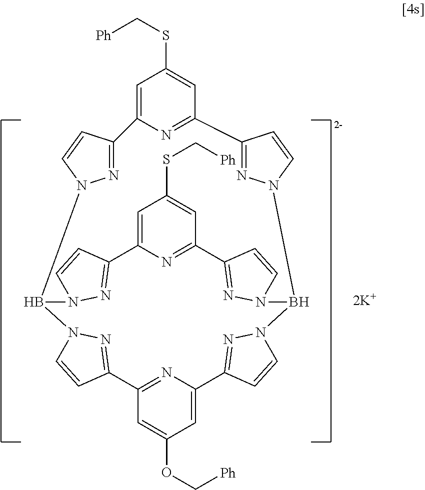

A Kontes flask was charged with sodium hydride (0.120 g, 5.00 mmol, 6.1 equiv) in 4 mL dry THF. Benzyl mercaptan (0.60 mL, 5.00 mmol, 6.1 equiv) was added under nitrogen. The solution was stirred overnight at 90° C. to form a solution of sodium benzyl thiolate. To this solution, was added bromo cage [4c] (0.800 g, 0.82 mmol, 1 equiv) in ca. 10 mL dry THF, and the solution was heated to 90° C. for 48 hours, resulting in a very cloudy yellow solution. The solution was cooled, the solvent evaporated under reduced pressure and the residue stirred in diethyl ether (100 mL) overnight. The insoluble solid was isolated by filtration and further washed with additional diethyl ether. The resulting pale yellow product was dried under vacuum. Yield: 0.630 g (70%). ¹H{¹¹B} NMR (500 MHz, dmso-d₆): δ 7.738 (6H, d, ³J_{HH}=2.8 Hz, 5′-pzH), 7.462 (6H, d, ³J_{HH}=8.0 Hz, o-Ph), 7.427 (6H, s, 3,5-pyH), 7.292 (6H, t, ³J_{HH}=8.0 Hz, m-Ph), 7.200 (3H, d, ³J_{HH}=8.0 Hz, p-Ph), 6.743 (6H, d, ³J_{HH}=2.8 Hz, 4′-pzH), 4.75 (2H, br m, BH), 4.460 (6H, s, CH₂S); ¹³C{¹H} NMR (125 MHz, dmso-d₆): δ 152.26 (2,6-pyC), 151.00 (3′-pzC), 148.40 (4-pyC-S), 136.88 (ipso-PhC), 136.04 (5′-pzCH), 128.91 (o-Ph), 128.49 (m-Ph), 127.21 (p-Ph), 114.85 (3,5-pyCH), 102.43 (4′-pzCHl), 34.13 (CH₂S); ¹¹B NMR (115.59 MHz, dmso-d₆): δ −2.08 (br s); ²³Na (132.33 MHz, dmso-d₆): δ −0.07 (a sodium signal is observed but ESI MS shows that this complex is predominantly the K⁺ salt. ESI-MS, neg. mode: [M⁺K⁺]⁻, [C₅₄H₄₁N₁₅B₂S₃K]⁻: m/z observed: 1056 amu. An X-ray crystal structure of Ba(cage [4s]), crystallized from diethyl ether, is shown in FIG. 3. The barium in this structure can be considered to be 9-coordinate (as shown)

### Example 30—Tris(2,6-pyrazolyl-4-(4′-methoxybenzylthiolato)pyridino)diborate dipotassium salt [4t]

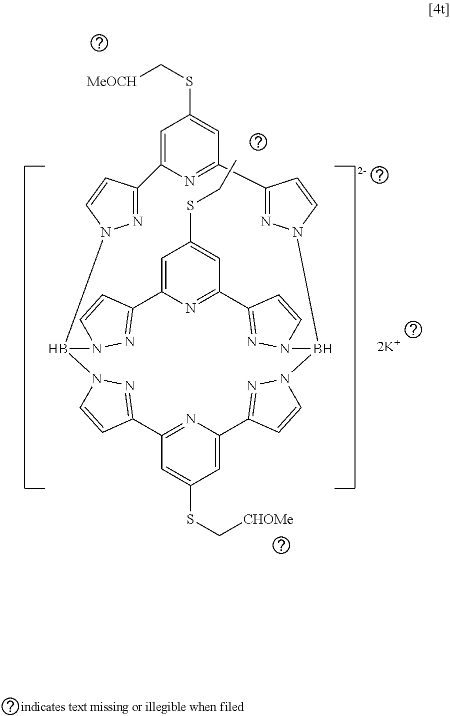

Compound [4t] was prepared by the same route as [4s] using 4-methoxybenzyl mercaptan (0.39 mL, 2.48 mmol, 6 equiv, 90% solution), NaH (0.060 g, 2.5 mmol, 6 equiv) and bromo-cage [4c] (0.400 g, 0.414 mmol, 1 equiv). The crude product was purified by stirring it in 100 mL diethyl ether overnight. The solid was then re-dissolved in acetone, filtered through Celite and the solvent removed under reduced pressure to give a yellow powder. Washing the solid with a minimum of benzene (it is somewhat soluble in this solvent) afforded a pale yellow solid of good purity. Yield: 0.260 g (53%). Mp.>300° C. ¹H{¹¹B} NMR (500 MHz, acetone-d₆:dmso-d₆ 10:1): δ 7.773 (6H, d, ³J_{HH}=2.8 Hz, 5′-pzH), 7.383-7.368 (12H, m, overlapping 2′-arylH and 3,5-pyH), 6.856 (6H, d, ³J_{HH}=6.0 Hz, 3′-arylH), 6.637 (6H, d, ³J_{HH}=2.8 Hz, 4′-pzH), 4.86 (2H, br s, BH), 4.341 (6H, s, CH₂S), 3.717 (9H, s, OCH₃); ¹¹B NMR (115.59 MHz, acetone-d₆:dmso-d₆ 10:1): δ −1.39 (br s).

### Example 31—Tris(2,6-pyrazolyl-4-(2′-(1″,3″-dioxo-2″-cyclopentyl)ethanethiolato)pyridino)diborate dipotassium salt [4u]

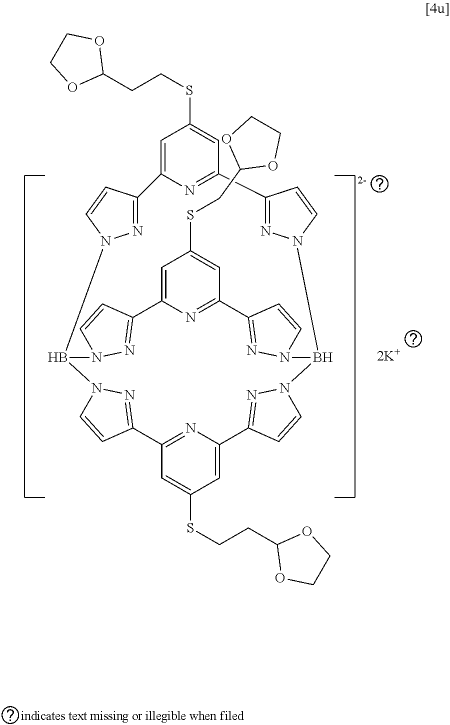

A Kontes flask equipped was charged with potassium hydride (0.0500 g, 1.24 mmol, 6 equiv) and 2 mL dry THF. To this, 1,3-dioxolane-2-ethanethiol (0.1653 g, 1.242 mmol, 6 equiv) was added under nitrogen. The solution was stirred overnight at 90° C. A solution of [4c](0.2000 g, 0.207 mmol, 1 equiv) in ca. 2 mL dry THF, was added to the Kontes flask and the solution was heated at 90° C. for 48 hours. The resulting deep wine-red solution was cooled, the solvent evaporated, and the residue stirred in 100 mL diethyl ether overnight. The suspension was filtered and the isolated solid was re-dissolved in acetone. This solution was filtered to remove a pale brown-red, insoluble byproduct and the filtrate evaporated to afford a wine-red solid product. Yield: 0.120 g (51%). ¹H{¹¹B} NMR (500 MHz, acetone-d₆): δ 7.782 (6H, d, ³J_{HH}=2.5 Hz, 5′-pzH), 7.404 (6H, s, 3,5-pyH), 6.708 (6H, d, ³J_{HH}=2.5 Hz, 4′-pzH), 4.960 (3H, t, ³J_{HH}=4.3 Hz, CH(OCH₂)), 4.89 (2H, br m, BH), 3.962 (6H, m, OCHₐH_{b}), 3.834 (6H, m, OCHₐH_{b}), 3.165 (6H, t, ³J_{HH}=7.8 Hz, SCH₂), 1.999 (6H, m, SCH₂CH₂); ¹³C{¹H} NMR (125 MHz, acetone-d₆): δ 153.89 (2,6-pyC), 152.62 (3′-pzC), 150.11 (4-pyC-S), 137.27 (5′-pzCH), 115.24 (3,5-pyCH), 103.15 (4′-pzCH), 102.55 (CH(OCH₂)₂), 65.49 (OCH₂), 34.20 (SCH₂CH₂), 25.13 (SCH₄₂); ¹¹B NMR (115.59 MHz, dmso-d₆): δ −1.29 (br s); ESI-MS, pos. mode: [M⁺3H⁺]+, [C₄₈H₅₀N₁₅B₂S₃O₆]⁺: m/z observed: 1050 amu (very weak).

### Example 32—Tris(2,6-pyrazolyl-4-(pyridonyl))diborate dipotassium salt [4v]

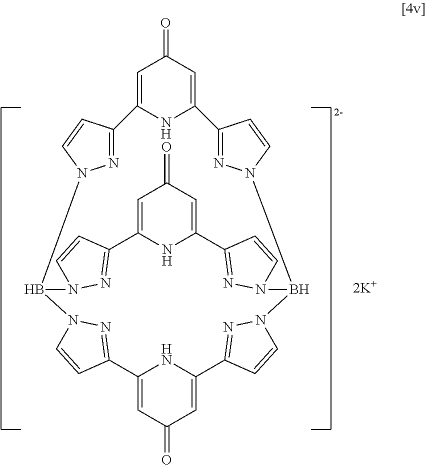

In a glovebox, a small Erlenmeyer flask was charged with N,N-dimethylethylenediamine (0.200 g, 2.27 mmol, 12 equiv), previously dried by distillation from BaO, and diglyme (10 mL). In a separate Erlenmeyer flask, benzyl potassium (0.294 g, 2.24 mmol, 12 equiv) was dissolved in diglyme (5 mL) and then added to the stirred solution of N,N-dimethylethylenediamine. The solution changed color from deep red to clear brown on mixing. A Kontes flask was charged with bromo cage [4c] (0.182 g, 0.188 mmol, 1 equiv) in ca. 10 mL dry diglyme and the contents transferred to the Kontes flask by Pasteur pipette, the flask sealed and removed from the glovebox. The solution was heated to 120° C. for 48 hours, resulting in a color change to pale orange with some insoluble beige precipitate. On cooling, the diglyme was removed by distillation under vacuum to afford a pale orange solid. The solid was suspended in THE (50 mL) in air and stirred for several hours. The solid was filtered off and the solid residue washed with additional THE and vacuum dried to afford an off-white to slightly orange powder. Yield: 0.137 g (89%). Mp.>300° C. ¹H{¹¹B} NMR (500 MHz, dmso-d₆): δ 7.730 (6H, d, ³J_{HH}=2.5 Hz, 5′-pzH), 6.461 (6H, d, ³J_{HH}=2.5 Hz, 4′-pzH), 6.352 (6H, s, 3,5-pyH), 4.70 (2H, br s, BH); ¹³C{¹H} NMR (125 MHz, dmso-d₆): δ 177.59 (4-pyC═O), 154.39 (2,6-pyC), 150.93 (3′-pzC), 137.02 (5′-pzCH), 111.28 (3,5-pyCH), 100.80 (4′-pzCH); ¹¹B NMR (115.59 MHz, dmso-d₆): δ −2.07 (br s). ¹H{¹¹B} NMR (500 MHz, D₂O): Major tautomer: 6 7.955 (6H, d, ³J_{HH}=2.5 Hz, 5′-pzH), 6.816 (6H, s, 3,5-pyH), 6.739 (6H, d, ³J_{HH}=2.5 Hz, 4′-pzH), 4.91 (2H, br s, BH); Minor tautomer (major:minor 6:1): δ 7.778 (6H, d, ³J_{HH}=2.5 Hz, 5′-pzH), 6.997 (6H, s, 3,5-pyH), 6.896 (6H, d, ³J_{HH}=2.5 Hz, 4′-pzH), BH resonance overlaps major tautomer; ¹³C{¹H} NMR (125 MHz, D₂O): Major tautomer: δ 138.52 (5′-pzCHl), 111.83 (3,5-pyCH), 103.60 (4′-pzCH); Minor tautomer: δ 135.19 (5′-pzCH), 112.12 (3,5-pyCH), 103.92 (4′-pzCH), quaternary C not observed in D₂O at this concentration; ¹¹B NMR (115.59 MHz, D₂O): 6+1.69 (s). ESI-MS, neg. mode: [M⁺K]⁻, [C₃₃H₂₃N₁₅B₂O₃K]⁻: m/z observed: 738 amu and [M]²⁻ [C₃₃H₂₃N₁₅B₂O₃]²⁻: m/z observed: 349.5 amu (base peak).

### Example 33a—Tri s(2,6-pyrazolyl-4-(methyl-1′-oxahept-5′E-enoate)pyridino)diborate dipotassium salt [4w]

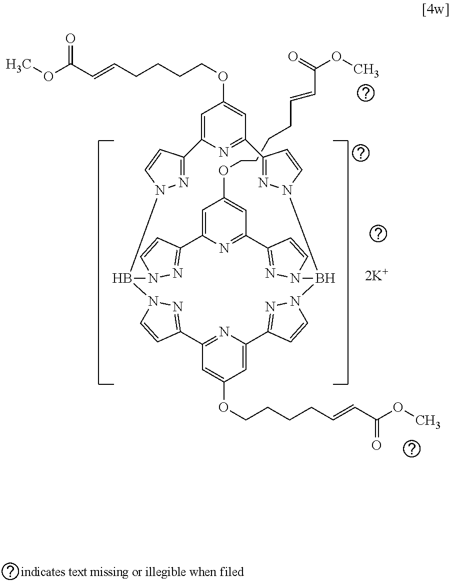

In a glove box, a Kontes flask was charged with [4k] (0.050 g, 0.049 mmol) and Grubbs-Hoveyda catalyst M720 (0.005 g, 12 mol %). The flask was removed from the glove box and placed under a nitrogen atmosphere. Dichloromethane (2 mL) solvent followed by methyl acrylate (0.015 mL, 0.16 mmol) in ca. 1 mL dichloromethane were added by syringe and the flask re-sealed. The dark green mixture was stirred at room temperature for 24 h resulting in a dark brown solution. The solvent was removed under reduced pressure. The residue was then extracted in acetone (10 mL), filtered and taken to dryness under reduced pressure. The residue was washed with diethyl ether followed by hexane and dried under vacuum to afford a beige solid. Yield: 0.035 g (60%). ¹H{¹¹B} NMR (500 MHz, acetone-d₆): δ 7.710 (6H, br s, 5′-pzH), 7.020 (6H, s, 3,5-pyH), 6.948 (3H, m, CH₂CH═), 6.595 (6H, br s, 4′-pzH), 5.868 (3H, d, ³J_{HH}=16 Hz, trans-7′-CH═CHₜᵣₐₙₛCO₂CH₃), 4.87 (2H, br s, BH), 4.136 (6H, y br t, 2′-OCH₂), 3.63 8 (9H, s, CO₂CH₃), 2.29 (6H, m, 5′-CH₂CH═), 1.816 (6H, pentet, ³J_{HH}=7.5 Hz, 3′-OCH₂CH₂), 1.661 (6H, pentet, ³J_{HH}=7.5 Hz, 4′-CH₂CH₂CH═); ¹¹B NMR (115.59 MHz, acetone-d₆): δ −1.28 (br s).

### Example 33b—Tris(2,6-pyrazolyl-4-(2′-aminoethan-1′-amino)pyridino)diborate dipotassium salt [4x]

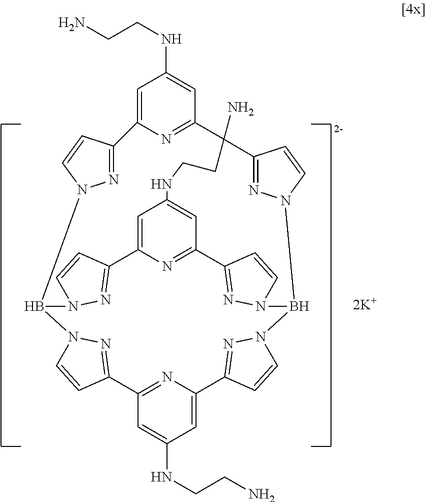

A Kontes flask was charged with 20 mL 1,2-diaminoethane (large excess), 0.250 g anhydrous potassium carbonate and K₂Br-Cage [4c] (0.500 g, 0.518 mmol) under nitrogen and sealed. The solution was heated to 140° C. for 24 hours. Cage [4c]dissolved as the temperature increased and the solution remained yellow in color. The flask was then cooled and the suspension was transferred to a round bottom flask, producing vapors on contact with air and becoming opaque and brown. The flask was heated to 50° C. and 1,2-diaminoethane was removed under reduced pressure. The solid product was dried under reduced pressure for 72 hours at 140° C. to remove residual 1,2-diaminoethane. The resulting solid was pale peach in color. Yield: 0.40 g (80%). ¹H{¹¹B} NMR (500 MHz, dmso-d₆): δ 7.675 (6H, d, ³J_{HH}=2.5 Hz, 5′-pzH), 6.702 (6H, s, 3,5-pyH), 6.511 (6H, d, ³J_{HH}=2.5 Hz, 4′-pzH), 6.235 (3H, t, 1′-NHCH₂), 4.72 (2H, br s, BH), 3.129 (6H, q, ³J_{HH}=5.0 Hz, 2′-NHCH₂), 2.717 (6H, t, ³J_{HH}=6.0 Hz, 5′-CH₂NH₂), 2.528 (6H, br s, CH₂NH₂); ¹³C{¹H} NMR (125 MHz, dmso-d₆): δ 154.91 (4-pyC), 152,73 (2,6-pyC), 152.18 (3′-pzC), 135.49 (5′-pzCH), 101.76 (3,5-pyCH), 101.37 (4′-pzCH), 45.51 (2″-NHCH₂), 41.00 (3″-CH₂NH₂); ¹¹B NMR (160.5 MHz, dmso-d₆): δ −1.8 (v br s).

### Example 33c—Tris(2,6-pyrazolyl-4-(3′-aminopropan-1′-amino)pyridino)diborate dipotassium salt [4y]

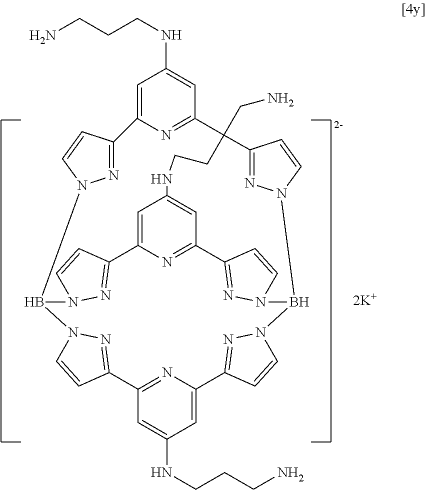

A Kontes flask was charged with 20 mL 1,3-diaminopropane (large excess), 0.250 g anhydrous potassium carbonate and K₂Br-Cage [4c] (0.500 g, 0.518 mmol) under nitrogen and sealed. The solution was heated to 140° C. for 24 hours. Cage [4c]dissolved as the temperature increased and the solution remained yellow in color. The flask was then cooled and the suspension was transferred to a round bottom flask, producing vapors on contact with air and becoming opaque and brown. The flask was heated to 50° C. and 1,3-diaminopropane was removed by trap-to-trap distillation under reduced pressure. The solid product was dried under reduced pressure for 48 hours at 100° C., then further dried for another 48 hours at 150° C. to remove residual 1,3-diaminopropane. The resulting solid was pale peach in color. Yield: 0.40 g (80%). ¹H{¹¹B} NMR (500 MHz, dmso-d₆): δ 7.659 (6H, d, ³J_{HH}=2.5 Hz, 5′-pzH), 6.685 (6H, s, 3,5-pyH), 6.487 (6H, d, ³J_{HH}=2.5 Hz, 4′-pzH), 6.224 (3H, t, 1′-NHCH₂), 4.71 (2H, br s, BH), 3.162 (6H, q, 2′-NHCH₂), 2.97 (6H, t, 5′-CH₂NH₂), 2.626 (6H, pentet, 4′-CH₂NH₂), 1.612 (6H, pentet, 3′-CH₂CH₂CH₂); ¹³C{¹H} NMR (125 MHz, dmso-d₆): δ 154.72 (4-pyC), 152,57 (2,6-pyC), 152.03 (3′-pzC), 135.30 (5′-pzCH), 101.15 (3,5-pyCH), 100.7 (4′-pzCH), 40.15 (2″-NHCH₂)*, 39.80 (4″-CH₂NH₂), 32.69 (3″-CH₂CH₂CH₂); ¹¹B NMR (160.5 MHz, dmso-d₆): δ −1.5 (br s).

### Example 33d—Tris(2,6-pyrazolyl-4-(N,N′-dimethyl-2′-aminoethan-1′-amino)pyridino)diborate dipotassium salt [4z]

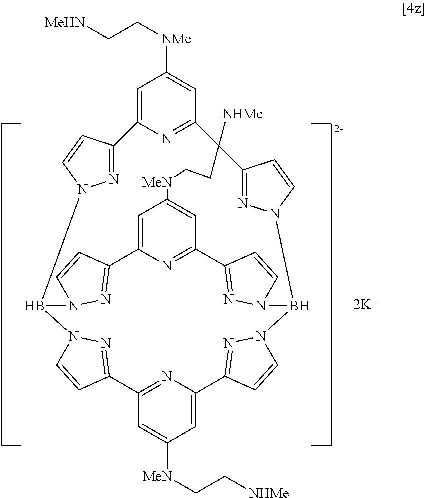

A similar procedure was used as for [4x]. Yield: 0.35 g (70%). ¹H{¹¹B} NMR (500 MHz, dmso-d₆): δ 7.694 (6H, d, ³J_{HH}=2.0 Hz, 5′-pzH), 6.787 (6H, s, 3,5-pyH), 6.658 (6H, d, ³J_{HH}=2.0 Hz, 4′-pzH), 4.74 (2H, br s, BH), 3.483 (6H, br t, ³J_{HH}=7.0 Hz, 2″-NMeCH₂), 3.011 (9H, s, 1″-NMeCH₂), 2.629 (6H, t, ³J_{HH}=7.0 Hz, 3″-CH₂NHMe₂), 2.280 (9H, s, 4″-CH₂NMeH); ¹³C{¹H} NMR (125 MHz, dmso-d₆): δ 154.34 (4-pyC), 152.81 (3′-pzC), 152.23 (2,6-pyC), 135.32 (5′-pzCH), 101.57 (4′-pzCH), 100.92 (3,5-pyCH), 50.57 (2″-NMeCH₂), 48.51 (3″-CH₂NMeH), 37.66 (1″-NMeCH₂), 4″-CH₂NMeH); ¹¹B NMR (160.5 MHz, dmso-d₆): δ −2.0 (v br s).

### Example 34—Tris(2,6-(5′-methylpyrazolyl)pyridino)diborate dipotassium salt [5a]·Me₂C═O·2H₂O

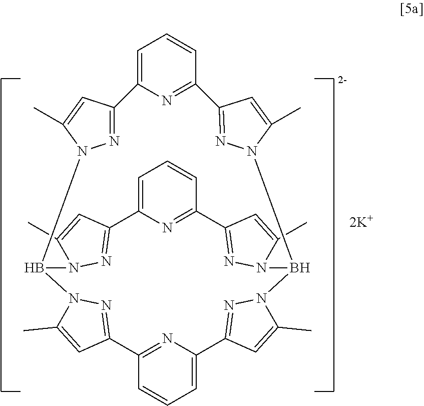

The 5′-methylpyrazolyl cage variant [5a] was prepared from arm [3′a] via the melt reaction shown in Scheme 5A. Cage [5a] was isolated as a discrete solvate containing 1 equiv of acetone and 2 equiv of H₂O by NMR integration, elemental analysis and X-ray crystallography. Yield of [5a]·Me₂C═O·2 H₂O on 2.8 g of arm scale was 1.05 g (30%). Mp.>300° C. ¹H NMR (500 MHz, acetone-d₆:dmso-d₆, 10:1): δ 7.604 (3H, t, ³J_{HH}=7.8 Hz, 4-pyH), 7.363 (6H, d, ³J_{HH}=7.8 Hz, 3,5-pyH), 6.371 (6H, s, 4′-pzH), 5.11 (2H, br m, BH), 3.10 (6H, br s, H₂O), 2.512 (18H, s, 5′-PzMe), 2.086 (6H, s, Me₂C═O); ¹³C{¹H} NMR (125 MHz): δ 154.17 (2,6-pyC), 150.71 (3′-pzC), 143.55 (5′-pzCMe), 137.06 (4-pyCH), 118.11 (3,5-pyCH), 103.38 (4′-pzCH), 13.57 (5′-pzMe); ¹¹B NMR (160.5 MHz): δ −8.0 (v. br s; just resolved as a d in pure acetone-d₆ with ¹J_{BH}=96 Hz); ¹⁵N NMR (indirect detection HMBC, δ relative to NH₃): δ 319.1 (2′-pzN), 295.0 (1-pyN), 242.7 (1′-pzN-B). Elem. analysis calcd. for [5a]·Me₂C═0.2 H₂O, C₄₂H₄₅N₁₅B₂O₃K₂: C, 55.57; H, 5.00; N, 23.15%. Found: C, 55.22; H, 5.03; N, 23.01%.

An X-ray crystal structure of the K₂ cage [5a] is shown in FIG. 4. The structure is a dimer comprising two cages, with one potassium in the middle of each cage, and one potassium between the arms on each cage, that then bridges through two acetones to the other cage.

### Example 35—tris(2, 6-di-(4′-bromopyrazolyl)pyridino)diborate dipotassium salt [5b]

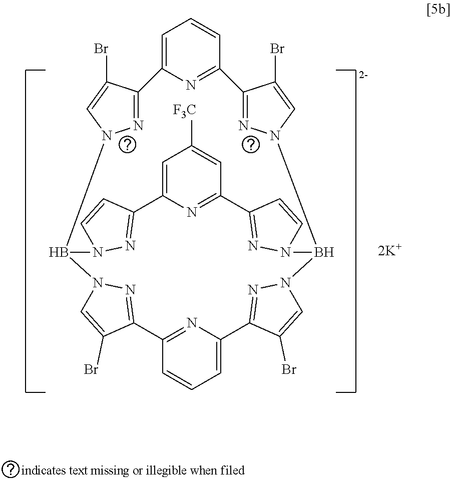

Solid [4a] (0.100 g, 0.137 mmol) was placed in a 50 mL round bottom flask and dissolved in 20 mL acetonitrile with stirring. A grain of benzoyl peroxide was added along with NBS (0.146 g, 0.823 mmol, 6 equiv) and the solution was irradiated with a 150W floodlight for 2 h. The white hexabromo product that formed was filtered off and washed with 100 mL acetonitrile. The filtrate was a mixture containing the hexabromo product [5b] as well as the octabromo and heptabromo products as the main constituent according to ESI MS (presumably by bromination of the 5′-position of some pyrazolyl rings). Yield: 1.10 g (36%). Mp.>300° C. ¹H NMR (500 MHz, dmso-d₆): δ 7.97 (6H, s, 5′-pzH), 7.93 (9H, overlapping m, 4-pyH and 3,5-pyH), 4.65 (2H, br m, BH); Negative Mode ESI MS: 1164 (C₃₃H₁₇N₁₅B₂Br₆K, molecular ion cluster for [5b]-K⁺).

### Example 36—tris(2, 6-di-(4′-trifluoromethylacetylpyrazolyl)pyridino)diborate dipotassium salt [5c]

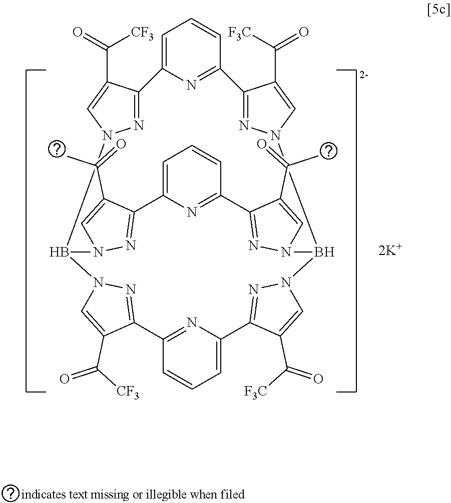

Cage [4a]·2 i-PrOH·2 H₂O (0.200 g, 0.226 mmol) was dissolved in dry pyridine (15 mL) and neat trifluoroacetic anhydride (ca. 25 equiv) was added to the solution by pipette resulting in fumes and formation of a pale orange solution. The reaction mixture was fitted with a condenser mounted with a drying tube and placed in an 80° C. oil bath. Stirring was continued at this temperature for 48 h resulting in formation of a very dark brown solution. The solvent was removed under reduced pressure and the brown solid was suspended in saturated NaHCO₃ (aq), stirred for 30 minutes and filtered. The dark brown solid was washed with another 50 mL of satd. NaHCO₃ (aq) followed by 100 mL of water. The filtrate was initially orange-brown but subsequent washings were colorless. After drying on a vacuum line, a dark brown solid was obtained that proved to be predominantly the hexakis trifluoroacetylation product with some of the pentakis in evidence. Yield (based on hexakis trifluoroacetylation): 0.248 g (84%). ¹H NMR (300 MHz, 3:1 acetone-d₆:dmso-d₆): δ 7.85 (6H, br s, 5′-pzH), 7.72 (3H, t, 4-pyH), 7.55 (6H, s, 3,5-pyH), 4.98 (2H, br s, BH), residual resonances are observed near 6.7 ppm due to unreacted 4′-pzH sites; ¹⁹F NMR (282.5 MHz): δ −72.56 was the major resonance although smaller resonances were observed at −72.37, -73.49 and -75.59, as well as many smaller resonances; “B NMR (96.3 MHz): δ −1.9 (v. br s). Negative Mode ESI MS: 1170 (C₄₃H₁₈N₁₅B₂KF₁₅O₅ cluster, pentakis trifluoroacetylation product); 1266 (C₄₅H₁₇N₁₅B₂KF₁₈O₆ cluster, hexakis trifluoroacetylation product). The ratio of hexakis:pentakis trifluoroacetylated products was ca. 100:75 by ESI MS

### Example 37—tris(2, 6-di-(4′-bromo-5′-methylpyrazolyl)pyridino)diborate dipotassium salt [5d]

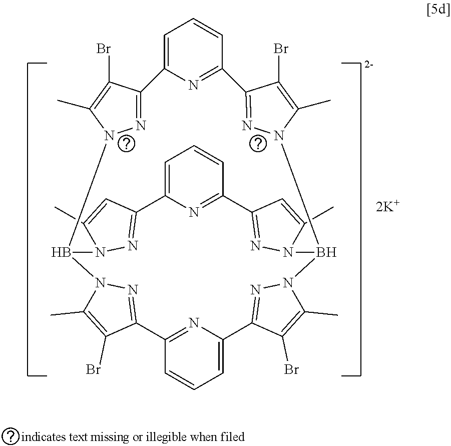

Solid [5a] (0.100 g, 0.123 mmol) was placed in a 50 mL round bottom flask and dissolved in 20 mL acetonitrile with stirring. A grain of benzoyl peroxide was added along with NBS (0.131 g, 0.738 mmol, 6 equiv) and the solution was irradiated with a 150W floodlight for 2 h. The white solid that formed was filtered off and washed with 100 mL acetonitrile. Yield: 0.155 g (98%). Mp.>300° C. ¹H NMR (500 MHz, dmso-d₆): δ 7.908 (3H, t, ³J_{HH}=7.8 Hz, 4-pyH), 7.810 (6H, d, ³J_{HH}=7.8 Hz, 3,5-pyH), 4.99 (2H, br m, BH), 2.276 (18H, s, 5′-pzCH₃); ¹³C{¹H} NMR (125 MHz): δ 151.36 (2,6-pyC), 146.29 (5′-pzCCH₃), 141.88 (3′-pzC), 136.67 (4-pyCH), 120.46 (3,5-pyCH), 91.09 (4′-pzCBr), 11.09 (5′-pzCH₃); ¹¹B NMR (160.5 MHz): δ −6.4 (v. br s); ¹⁵N NMR (indirect detection HMBC, δ relative to NH₃): δ 306.3 (1-pyN), 236.1 (1′-pzN-B); 2′-pzN not observed. Negative Mode ESI MS: 1248 (C₃₉H₂₉N₁₅B₂Br₆K, molecular ion cluster for [5d]—K⁺).

### Example 38—Preparation of Clathrochelate Ligand, K₂ Cage [6a]

### 1,3-((N-dimethylamino)-(prop-2-en-1-one))benzene [13]

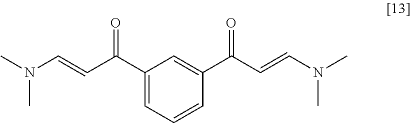

A 250 mL round bottom flask was charged with 5.00 g (30.6 mmol) 1,3-diacetylbenzene and 125 mL toluene. To this solution was added 12.2 mL (91.9 mmol, 3 equiv) of N,N-dimethylformamide dimethyl acetal and the reaction mixture was refluxed for 24 h. The reaction flask was transferred to the fridge (−5° C.). The precipitate was isolated by and washed with hexane to yield 1.79 g of [13] as pale yellow crystals. A second crop of crystals was isolated by adding hexane until precipitation just began followed by cooling at −5° C. Total yield: 3.35 g (12.2 mmol, 41%). Mp. 160-164° C. ¹H NMR (500 MHz, CDCl₃): δ 8.38 (1H, s, 2-arylH), 7.98 (2H, dd, J_{HH}=8.5, 1.8 Hz, 4,6-arylH), 7.79 (2H, d, ³J_{HH}=12.5 Hz, N—CH═CH), 7.44 (1H, t, ³J_{HH}=8.5 Hz. 5-arylH), 5.76 (2H, d, ³J_{HH}=12.5 Hz, N—CH═CH), 3.13 (6H, br s, NCH₃), 2.91 (6H, br s, NCH₃); ¹³C{¹H} NMR (125 MHz, CDCl₃): δ 188.38 (C═O), 154.49 (═CH—N), 140.52 (1,3-arylC), 130.04 (4,6-arylCH), 128.11 (5-arylCH), 126.56 (2-arylCH), 91.76 (CH═CH—N), 45.19 (NMe), 37.45 (NMe).

### 1,3-bis(pyrazolyl)benzene—mixture of tautomers [14]

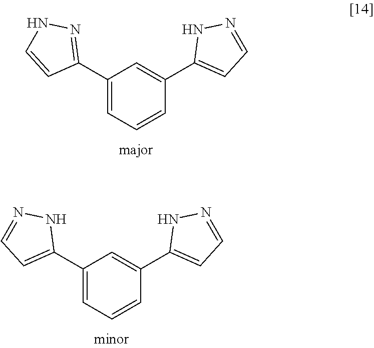 [SMILES: c1cc(-c2cc[nH]n2)cc(-c2ccn[nH]2)c1 ; c1cc(-c2ccn[nH]2)cc(-c2ccn[nH]2)c1]

A 250 mL round bottom flask was charged with 1.75 g (6.43 mmol) of [13] and 100 mL anhydrous ethanol. Hydrazine monohydrate was added (1.00 mL, 19.3 mmol, 3 equiv) and the mixture refluxed at 80° C. for 48 hours. The solvent was removed under reduced pressure to leave a tacky colorless oil. Diethyl ether (150 mL) was added to the oil and a wide solid slowly formed. The suspension was stirred for 2 hours and the precipitate was isolated by vacuum filtration and washed with diethyl ether to yield 1.15 g (5.47 mmol, 85%) of [14] as a white, microcrystalline powder. The compound consisted of a mixture of 1-(pyrazol-3′-yl)-3-(pyrazol-5′-yl)benzene (major isomer) and 1,3-di(pyrazol-3′-yl)benzene (minor isomer) in 1.1:1 ratio. Mp. 120-124° C. ¹H NMR (500 MHz, dmso-d₆): δ 13.36 (1H, br s, major pyrazol-5′-yl-NH), 12.91 (3H, br s, overlapping major pyrazol-3′-yl-NH and minor pyrazol-3′-yl-NH), 8.26 (1H, br s, major 2-arylH), 8.19 (1H, br s, minor 2-arylH), 7.42-7.80 (10H, multiple br m, overlapping major and minor 4-, 5-, 6-arylH and 5′-pzH), 6.77 (4H, br m, 4′-pzH). ¹³C{¹H} NMR (125 MHz, dmso-d₆) δ 150.10 (3′-pzC), 134.11 (1,3-arylC), 129.89 (4,6-arylCH), 128.87 (5-arylCH), 124.16 (5′-pzCH), 121.86 (2-arylCH), 101.90 (4′-pzCH); assignments tentative, presumably representing the minor (symmetrical) isomer.

### tris(1,3-dipyrazolylbenzene)diborate dipotassium salt [6a]

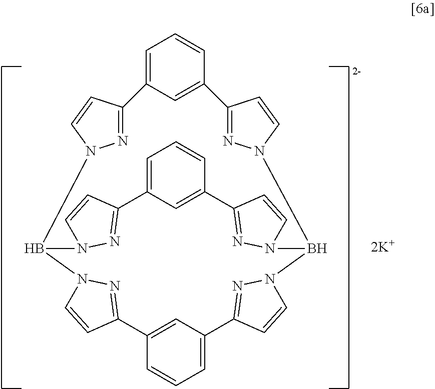

Solid [14] (0.104 g, 0.495 mmol, 3 equiv) and KBH₄ (0.0178 g, 0.330 mmol, 2 equiv) were ground into a fine powder. The solid mixture was added to a small test tube and then immersed in a silicone oil bath. The temperature was slowly raised to 250° C. The solids softened around 150° C. and melted with outgassing between 175-190° C. Evolution of H₂ ceased as the temperature reached 275° C. The test tube was removed from the bath and cooled to room temperature. Extraction with acetone dissolved most of the solid and the insoluble fraction was removed by filtration. Yield of K₂ cage [6a]: 0.065 g (63%). Mp.>300° C. ¹H NMR (500 MHz, acetone-d₆): δ 8.39 (3H, s, 2-arylH), 7.79 (6H, d, ³J_{HH}=7.5 Hz, 4,6-arylH), 7.74 (6H, br s, 5′-pzH), 7.44 (3H, t, ³J_{HH}=7.5 Hz, 5-arylH), 6.76 (6H, br s, 4′-pzH), 5.14 (2H, br s, BH); ¹³C{¹H} NMR (125 MHz, acetone-d₆): δ 136.43 (4,6-arylCH), 129.52 (5-arylCH), 125.06 (5′-pzCHl), 122.98 (2-arylCH), 102.51 (4′-pzCHl), quaternary C not observed; ¹¹B NMR (160.51 MHz, acetone-d₆): δ 1.58 (v br s). ¹H NMR (500 MHz, dmso-d₆): δ 8.25 (3H, br s, 2-arylH), 7.72 (6H, dd, ³J_{HH}=7.5 Hz, ⁴J_{HH}=2.0 Hz, 4,6-arylH), 7.71 (6H, d, 5′-pzH), 7.42 (3H, t, ³J_{HH}=7.5 Hz, 5-arylH), 6.74 (6H, br s, 4′-pzH), BH not observed due to broad H₂O resonance; ¹³C{¹H} NMR (125 MHz, dmso-d₆): δ 147.62 (3′-pzC), 133.23 (1,3-arylC), 133.15 (4,6-arylCH), 129.00 (5-arylCH), 123.99 (5′-pzCHl), 121.79 (2-arylCH), 101.89 (4′-pzCHl); ¹¹B NMR (160.51 MHz, dmso-d₆): δ 1.23 (v br s).

### Example 39a—Preparation of Clathrochelate Ligand, K₂ Cage [7a]

### 1,3-((N-dimethylamino)-(prop-2-imino-1-one))benzene [15]

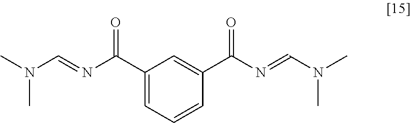

A 1 L round bottom flask was charged with isophthalamide (10.00 g, 60.90 mmol) and 500 mL THF. To this solution was added 32 mL (241 mmol, ca. 4 equiv) of N,N-dimethylformamide dimethyl acetal and the reaction mixture refluxed for 24 h. The starting material slowly dissolved but after a few hours a new white precipitate began to deposit. The reaction mixture was cooled to room temperature and the white precipitate was filtered off. After air drying, the product [15] was isolated as a chalky white powder (13.51 g, 49.25 mmol, 81% yield). Mp. 200-203° C. ¹H NMR (500 MHz, dmso-d₆): δ 8.92 (1H, t, ⁴J_{HH}=1.5 Hz, 2-arylH), 8.63 (2H, s, CH═N), 8.29 (2H, dd, ³J_{HH}, ⁴J_{HH}=7.8, 1.5 Hz, 4,6-arylH), 7.52 (1H, t, ³J_{HH}=7.8 Hz, 5-arylH), 3.21 (6H, br s, NCH₃), 3.16 (6H, br s, NCH₃); ¹³C{¹H} NMR (125 MHz, dmso-d₆): δ 175.51 (C═O), 160.91 (CH═N), 136.87 (1,3-arylC), 132.39 (4,6-arylC), 130.20 (2-arylC), 127.86 (5-arylC), 40.95 (NMe), 34.98 (NMe).

### 2,6-di(1′,2′,4′-triazol-3′-yl)benzene [16]

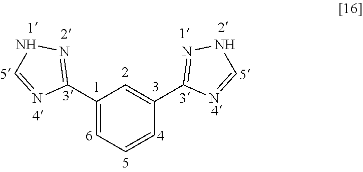 [SMILES: c1cc(-c2nc[nH]n2)cc(-c2nc[nH]n2)c1]

(General numbering scheme for compounds with this linkage is shown here)

1,3-bis(N,N-dimethylamino-2-imino-3-one) benzene, [15], (2.00 g, 7.29 mmol) was dissolved in glacial acetic acid (30 mL). Hydrazine monohydrate (0.81 mL, 16.7 mmol) was added and the solution changed from clear and colorless to opaque white. The solution was heated to 120° C. and stirred at reflux for 3 hours. As the solution heated, it changed to a transparent colorless solution. The solution was cooled to room temperature and diluted with ethyl acetate and then neutralized with saturated sodium bicarbonate solution. After the solution no longer produced CO₂, the aqueous layer was removed by separation and the organic layer was washed with distilled H₂O and brine. The organic layer was dried over MgSO₄, filtered and the solvent removed under reduced pressure to yield white powder as the crude product. The remaining product precipitated out of the aqueous layer and was isolated by vacuum filtration. Yield of [16]: 1.46 g (94%). Mp: 284-287° C. ¹H NMR (500 MHz, dmso-d₆): δ 14.26 (2H, br s, NH), 8.73 (1H, s, 2-arylH), 8.52 (2H, br s, 5′-TrH), 8.06 (2H, d, 4,6-arylH), 7.60 (1H, t, 5-arylH); ¹³C{¹H} NMR (125 MHz, dmso-d₆) δ 158.55 (3′-TrC), 146.76 (5′-TrCH), 130.76 (5-arylCH), 129.38 (1,3-arylC), 126.50 (4,6-arylCH), 123.45 (2-arylCH).

### tris(2,6-di(1′,2′,4′-triazol-3′ yl)benzene)diborate dipotassium salt [7a]

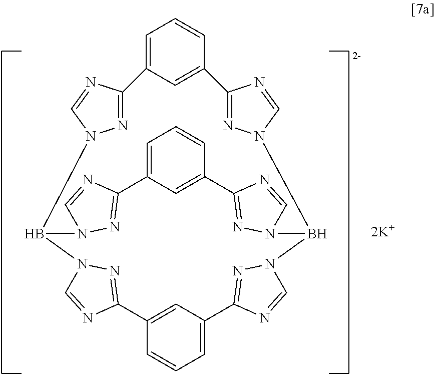

Solid [16] (0.5124 g, 2.41 mmol, 3 equiv) and KBH₄ (0.0870 g, 1.62 mmol, 2 equiv) were ground into a fine powder using a mortar and pestle. The solid mixture was added to a small test tube and then immersed in a silicone oil bath. The temperature was slowly raised to 290° C. The solids softened around 250° C. and partially melted between 280-290° C. Evolution of H₂ was not very clear in this case so the reaction mixture was heated at 295° C. for about 15 minutes. The test tube was removed from the bath and cooled to room temperature. The solid crystalline residue would not dissolve in acetone but dissolved with mild heating in dmso-d₆ for NMR investigation. Yield of K₂ cage [7a]: 0.505 g (87%). Mp.>300° C. ¹H NMR (300 MHz, dmso-d₆): δ 8.75 (3H, t, ⁴J_{HH}=1.5 Hz, 2-arylH), 8.22 (6H, s, 3′-pzH), 7.99 (6H, dd, ³J_{HH}=7.5 Hz, ⁴J_{HH}=1.5 Hz, 4,6-arylH), 7.46 (3H, t, ³J_{HH}=7.5 Hz, 5-arylH), 6.71 (1OH, v. br, coordinated H₂O), no BH resonance was observed; ¹³C{¹H} NMR (75 MHz, dmso-d₆): δ 159.02 (5′-TrC), 148.29 (3′-TrCH), 132.29 (1,3-arylC), 128.71 (5-arylCH), 125.24 (4,6-arylCH), 123.04 (2-arylCH); ¹¹B NMR (160.51 MHz, dmso-d₆): δ 1.45 (v br s).

### Example 39b—Preparation of Clathrochelate Ligand, K₂ Cage [7′a]

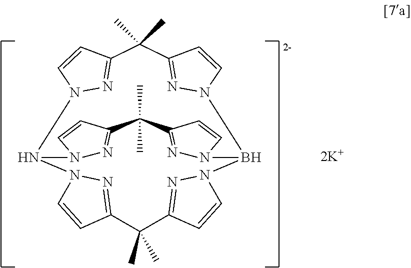

Cage [7′a] was prepared in diglyme solution. Diglyme (20 mL) was added to solid arm [3g] (0.400 g, 2.27 mmol, 3 equiv), potassium borohydride, KBH₄ (0.082 g, 1.52 mmol, 2 equiv) and Y(OTf)₃ (0.024 g, 2 mol %) in a Kontes flask in a nitrogen glovebox. The flask was sealed, removed from the glovebox and heated in an oil bath at 162° C. for 48 h. The solution was cooled, the diglyme removed under reduced pressure and the residue dissolved in acetone. Filtration and removal of solvent left a glassy beige semi-solid. This was extracted into diethyl ether, filtered and the solvent removed under reduced pressure to afford an off-white powder. Yield: 0.365 g (75% based on cage [3g]). ¹H{¹¹B} NMR (500 MHz, dmso-d₆:acetone-d₆): δ 7.435 (6H, br s, 5′-pzH), 6.115 (6H, br s, 4′-pzH), 4.73 (2H, br s, BH), 1.492 (18H, s, CMe₂); ¹¹B NMR (160.5 MHz, acetone-d₆): δ −3.04 (d, ¹J_{BH}=104 Hz.

### Example 40a—Bis(2,6-pyrazolylpyridino)diborate dipotassium salt, [8a]

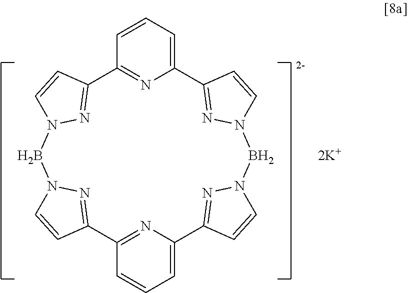

Reaction of 2,6-dipyrazolylpyridine arm [3a] with KBH₄ in 1:1 ratio using Y(OTf)₃ (2 mol %) as catalyst on a 1.25 g scale afforded about 0.780 g of ring [8a]after desolvation under vacuum. Yield: 50%. Mp.>300° C. ¹H NMR (500 MHz, acetone-d₆): δ 7.661 (2H, t, ³J_{HH}=7.8 Hz, 4-pyH), 7.553 (4H, d, ³J_{HH}=2.0 Hz, 5′-pzH), 7.441 (4H, d, ³J_{HH}=7.8 Hz, 3,5-pyH), 6.557 (4H, d, ³J_{HH}=2.0 Hz, 4′-pzH), 3.80 (4H, br s, BH₂); ¹³C{¹H} NMR (125 MHz, acetone-d₆): δ 154.17 (2,6-pyC), 152.11 (3′-pzC), 137.96 (4-pyCH), 136.43 (5′-pzCHl), 117.83 (3,5-pyCH), 102.30 (4′-pzCH); ¹¹B NMR (160.51 MHz, acetone-d₆): δ −6.40 (v. br t, ¹J_{11B-H} Ca. 86 Hz, BH₂, coupling not fully resolved); ESI-MS, neg. mode: [C₂₂H₁₈N₁₀B₂K]⁻, m/z observed: 483 amu.

### Example 40b—Bis(2,2-dipyrazolylpropane)diborate dipotassium salt, [8′a]

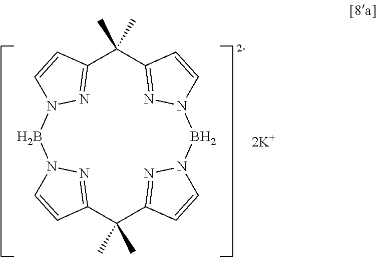

Reaction of arm [3g] with KBH₄ in 1:1 stoichiometry using Y(OTf)₃ (2 mol %) as catalyst afforded the ring [8′a], but contaminated with cage [7′a] and unknown tetrakis(pyrazolyl)borates identified by sharp resonances in the ¹¹B NMR between −1 and +3 ppm. The reaction carried out using 0.400 g arm (2.27 mmol) and 0.122 g KBH₄ (2.27 mmol) with 0.024 g Y(OTf)₃ (2 mol %) afforded about 0.35 g of an off-white powder. The ring product was notably less soluble in diethyl ether than the corresponding cage [7′a]. Yield: 67% (based on the ring structure [8′a]). ¹H{¹¹B} NMR (500 MHz, acetone-d₆): δ 7.403 (4H, d, ³J_{HH}=2.0 Hz, 5′-pzH), 6.026 (4H, d, ³J_{HH}=2.0 Hz, 4′-pzH), 2.92 (4H, br s, BH₂), 1.547 (12H, s, CMe₂); ¹¹B NMR (160.5 MHz, acetone-d₆): δ −6.96 (br t, ¹J_{BH}=88 Hz, BH₂ coupling not completely resolved).

### Example 40c—Bis(μ-N1,N1-2,2-dipyrazolylpropane)-bis(N2,N2-2,2-dipyrazolylpropane)diborate dipotassium salt, [8″a]

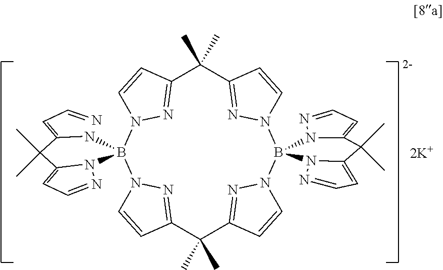

The dispiro-diborate ring [8″a] was prepared in the melt using 2,2-dipyrazolylpropane arm [3g] (0.4012 g, 2.277 mmol, 2 equiv) and potassium borohydride, KBH₄ (0.0614 g, 1.14 mmol, 1 equiv) ground into a fine powder using a mortar and pestle. The solid mixture was stirred under vacuum for 10 min then heated under nitrogen with the temperature increasing from about 85° C. to 200° C., over about 30 minutes. The solid mixture melted at about 130-150° C. but eventually re-solidified at about 200° C. Once the temperature had reached 200° C., this temperature was maintained for 2 h to give a voluminous beige glassy solid. On cooling, the flask was opened and the residue dissolved in acetone, filtered and the solvent removed under reduced pressure. Yield: 0.366 g (81%). Mp.>300° C. ¹H NMR (500 MHz, acetone-d₆): δ 7.498 (4H, br s, 5′a-pzH), 6.948 (4H, br s, 5′b-pzH), 6.224 (4H, br s, 4′a-pzH), 6.116 (4H, d, ³J_{HH}=3.0 Hz, 4′b-pzH), 1.492 (12H, s, CMe₂a), 1.438 (12H, s, CMe₂b); ¹³C{¹H} NMR (125 MHz, acetone-d₆): δ 161.88 (3′b-pzC), 152.34 (3′a-pzC), 141.44 (5′a-pzCH), 135.29 (5′b-pzCH), 102.41 (4′b-pzCH), 100.67 (4′a-pzCH), 37.08 (CMe₂b), 35.04 (CMe₂a), 32.68 (CMe₂a), 31.33 (CMe₂b); ¹¹B NMR (160.51 MHz, acetone-d₆): δ −0.78 (s, B); a=N1,N1-bridging dipyrazolylpropane and b=N2,N2-terminal 2,2-dipyrazolylpropane. ESI-MS, neg. mode: [C₃₆H₄₀N₁₆B₂K]⁻, m/z observed (%): 755 (6), 756 (44), 757 (100), 758 (48), 759 (18), 760 (6); calculated (%): 755 (5), 756 (42), 757 (100), 758 (44), 759 (16), 760 (4) amu. An X-ray crystal structure of [8″a], crystallized from acetone, is shown in FIG. 11. It includes four Me₂Cpz₂ units bond to two borons, to form a triple ring structure. The two potassium counterions are bonded η-5 to one pyrazole ring, and then through bridging water and acetone to a potassium in the next unit, so this becomes a linear polymer. The inner 16-membered ring has 4 N atoms available to bind a metal.

### Example 41—Bis(2,6-pyrazolyl-4-chloropyridino)diborate dipotassium salt, [8b]0.5 diglyme solvate

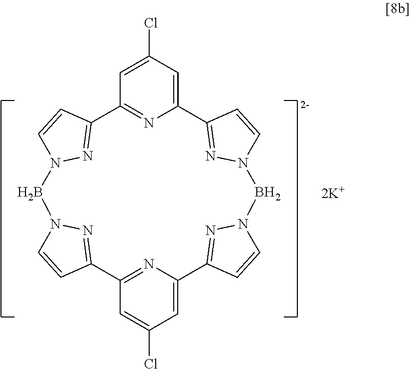

Reaction of the 2,6-dipyrazolyl-4-chloropyridine arm [3b] with KBH₄ using Y(OTf)₃ (2 mol %) catalyst afforded a good yield of the ring product in reasonable purity. The reaction carried out on 0.505 g scale afforded about 0.355 g of ‘chloro ring’ [8b]. Yield: 53%. Mp.>300° C. ¹H NMR (500 MHz, acetone-d₆): δ 7.558 (4H, d, ³J_{HH}=2.5 Hz, 5′-pzH), 7.476 (4H, s, 3,5-pyH), 6.639 (4H, d, ³J_{HH}=2.5 Hz, 4′-pzH), 3.80 (4H, br s, BH₂), 3.555 (2H, t, diglyme, CH₂OCH₂), 3.463 (2H, t, diglyme, CH₂OCH₃), 3.282 (3H, s, diglyme, OCH₃); ¹³C{¹H} NMR (125 MHz, acetone-d₆): δ 155.98 (2,6-pyC), 151.05 (3′-pzC), 144.96 (4-pyC-Cl), 136.56 (5′-pzCH), 117.38 (3,5-pyCH), 102.87 (4′-pzCH), 72.69 (CH₂OCH₃), 71.07 (CH₂OCH₂), 58.64 (OCH₃); ¹¹B NMR (160.51 MHz, acetone-d₆): δ −6.44 (v. br s, BH₂ coupling not resolved); ESI-MS, neg. mode: [C₂₂H₁₆N₁₀B₂Cl₂K]⁻, m/z observed: 551 amu.

### Example 42—Bis(2,6-pyrazolyl-4-bromopyridino)diborate dipotassium salt, [8c]bis(diglyme) solvate

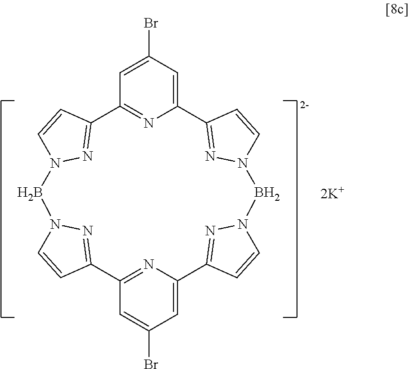

Reaction of the 2,6-dipyrazolyl-4-bromopyridine arm [3c] with KBH₄ using Y(OTf)₃ (2 mol %) catalyst afforded a good yield of the ring product in reasonable purity. The reaction carried out on 0.500 g arm scale afforded about 0.455 g of ‘bromo ring’ [8c]. Yield: 56%. Mp. dec.>294° C. ¹H{¹¹B} NMR (500 MHz, acetone-d₆): δ 7.630 (4H, s, 3,5-pyH), 7.554 (4H, d, ³J_{HH}=2.2 Hz, 5′-pzH), 6.637 (4H, d, ³J_{HH}=2.2 Hz, 4′-pzH), 3.79 (4H, br s, BH₂), 3.554 (8H, t, diglyme, CH₂OCH₂), 3.463 (8H, t, diglyme, CH₂OCH₃), 3.282 (12H, s, diglyme, OCH₃); ¹³C{¹H} NMR (125 MHz, acetone-d₆): δ 155.84 (2,6-pyC), 150.88 (3′-pzC), 136.57 (5′-pzCH), 133.72 (4-pyC-Br), 120.39 (3,5-pyCH), 102.89 (4′-pzCH), 72.69 (CH₂OCH₃), 71.08 (CH₂OCH₂), 58.85 (OCH₃); ¹¹B NMR (160.51 MHz, acetone-d₆): δ −6.46 (v. br s, BH₂ coupling not resolved); ESI-MS, neg. mode: [C₂₂H₁₆N₁₀B₂Br₂K]⁻, m/z observed: 641 amu.

### Example 43—Bis(2,6-pyrazolyl-4-iodopyridino)diborate dipotassium salt, [8d]tris(diglyme) solvate

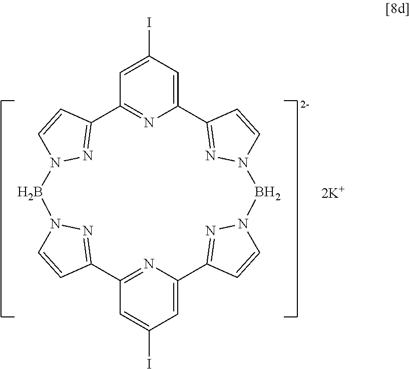

Reaction of the 2,6-dipyrazolyl-4-iodopyridine arm [3d] with KBH₄ using Y(OTf)₃ (2 mol %) catalyst afforded a good yield of the ring product in reasonable purity. The reaction carried out on 0.400 g arm scale afforded about 0.255 g of ‘iodo ring’ [8d]. Yield: 37%. Mp. dec. >252° C. ¹H NMR (500 MHz, acetone-d₆): δ 7.830 (4H, s, 3,5-pyH), 7.537 (4H, d, ³J_{HH}=2.5 Hz, 5′-pzH), 6.607 (4H, d, ³J_{HH}=2.0 Hz, 4′-pzH), 3.80 (4H, br s, BH₂), 3.554 (12H, t, diglyme, CH₂OCH₂), 3.463 (12H, t, diglyme, CH₂OCH₃), 3.282 (18H, s, diglyme, OCH₃); ¹³C{¹H} NMR (125 MHz, acetone-d₆): δ 155.28 (2,6-pyC), 150.65 (3′-pzC), 136.55 (5′-pzCHl), 126.49 (3,5-pyCH), 106.54 (4-pyC-I), 102.80 (4′-pzCH), 72.69 (CH₂OCH₃), 71.08 (CH₂OCH₂), 58.85 (OCH₃); ¹¹B NMR (160.51 MHz, acetone-d₆): δ −6.30 (v. br s, BH₂ coupling not resolved); ESI-MS, neg. mode: [C₂₂H₁₆N₁₀B₂I₂K]⁻, m/z observed: 735 amu.

### Example 44—Bis(2,6-pyrazolyl-4-cyanopyridino)diborate dipotassium salt, [8e] bis(diglyme) solvate

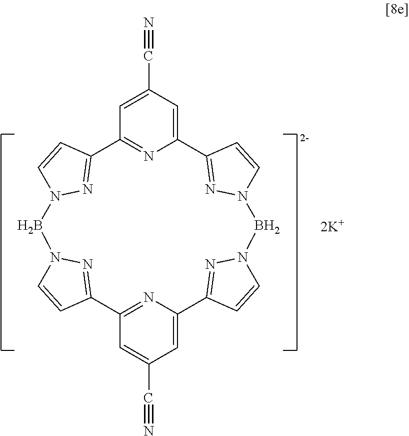

Reaction of the 2,6-dipyrazolyl-4-cyanopyridine arm [3e] with KBH₄ using Y(OTf)₃ (2 mol %) catalyst afforded a good yield of the ring product in reasonable purity. The reaction carried out on 0.354 g arm scale afforded about 0.310 g of ‘cyano ring’ [8e] as a bis(diglyme) solvate. Yield: 49%. Mp.>300° C. ¹H NMR (500 MHz, acetone-d₆): δ 7.776 (4H, s, 3,5-pyH), 7.610 (4H, d, ³J_{HH}=2.0 Hz, 5′-pzH), 6.751 (4H, d, ³J_{HH}=2.0 Hz, 4′-pzH), 3.82 (4H, br s, BH₂); ¹³C{¹H} NMR (125 MHz, acetone-d₆): δ 155.37 (2,6-pyC), 150.54 (3′-pzC), 136.64 (5′-pzCH), 121.85 (CN), 118.82 (3,5-pyCH), 117.99 (4-pyC-CN), 103.23 (4′-pzCH), 72.69 (CH₂OCH₃), 71.08 (CH₂OCH₂), 58.85 (OCH₃); ¹¹B NMR (160.51 MHz, acetone-d₆): δ −6.40 (v. br s, BH₂ coupling not resolved); ESI-MS, neg. mode: [C₂₄H₁₆N₁₂B₂K]⁻, m/z observed: 533 amu.

### Example 45—Preparation of Metal Complexes of Clathrochelate Ligand, Cage [4a]

A general procedure that was used to prepare metal complexes of a clathrochelate ligand, such as cage [4a], is described here. Commercial metal salts were used. Generally nitrate salts (usually as hydrates) were chosen in order to ensure solubility, however, in some cases other suitable metal salts such as triflates (CF₃SO₃⁻) can be used instead of nitrates. A solution of ca. 0.100 g (0.12 mmol) of K₂ cage [4a]·i-PrOH·2H₂O in 3 mL acetone was prepared. The metal salt was weighed separately into a 25 mL vial and dissolved in 5 mL of acetone. In most cases, to prepare mononuclear (1:1) metal complexes the reactions were carried out using <1 up to about 3.5 equivalents of metal salt. To prepare trimetallic (3:1) metal complexes the reactions were carried out using greater than 3.5 equivalents of metal salt. The colorless solution of K₂ cage [4a] in acetone was added dropwise by pipette to the stirred metal salt solution. In most cases, the metal-cage complex precipitated immediately, or within a few minutes, as a fine solid. The reaction mixture was stirred for at least 3 h. In a few cases, the metal nitrate was only sparingly soluble in acetone so about 20% water was added to the stirred mixture resulting in precipitation of the cage complex. The metal complex was separated by centrifugation of the suspension followed by washing of the solid with acetone and vacuum drying for at least 1 h.

In the case of Cd(NO₃)₂·4 H₂O, a mixture of K₂ cage [4a] and Cd cage [4a] was obtained unless a large excess of Cd(NO₃)₂·4 H₂O was used (ca. 20 equiv). For Ce³⁺, K₂ cage [4a] was reacted with 1 equiv. of the orange Ce⁴⁺ salt, (NH₄)₂[Ce(NO₃)₆)] in acetone; reduction to Ce³⁺ was evident from the fade in color to white and the paramagnetic NMR spectrum of the product. It is speculated that oxidation of isopropanol present in the K₂ cage [4a] and/or in trace amounts in the acetone solvent may result in reduction of Ce⁴⁺ to Ce³⁺ as the reaction proceeds. The reaction suspensions were stirred for at least 18 h, transferred to a 15 mL centrifuge tube and centrifuged to isolate the precipitate. The supernatant was decanted off and the solid washed with acetone (10 mL) and centrifuged down. This washing procedure was repeated three times and the final washings contained no color to indicate the presence of metal ions.

The paramagnetic Co²⁺ and Ni²⁺ monometallic complexes displayed lower symmetry at room temperature in dmso-d₆, as expected, but at high temperature, a symmetrical spectrum is obtained consistent with metal migration between binding sites or dissociative exchange; diamagnetic Zn²⁺, Y³⁺ and La³⁺ all showed symmetrical spectra at room temperature.

Co Cage [4a]·3H₂O=Metal Complex [9a]:

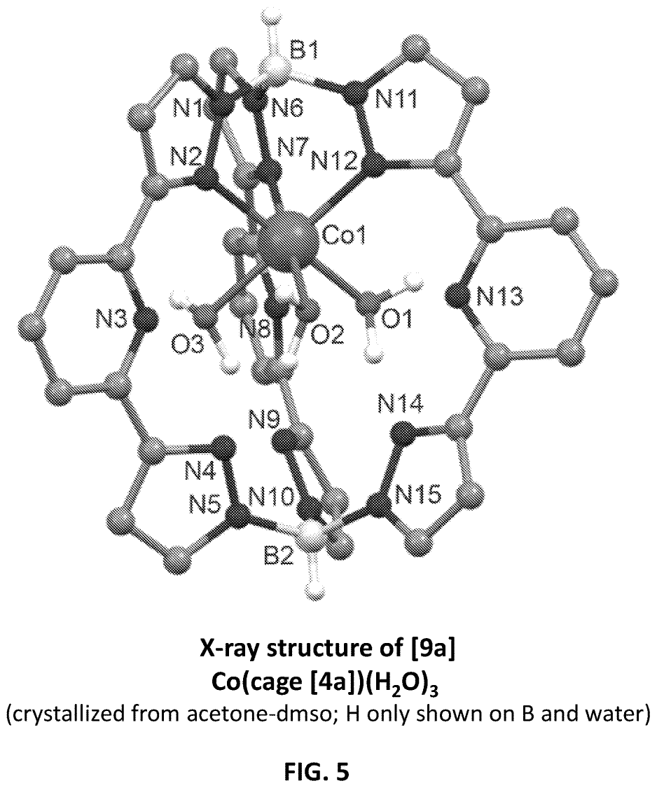

Mauve powder. Yield: 92% (based on formula). Mp. dec. 315° C. ¹H NMR (300 MHz, dmso-d₆, 298 K): δ 80.0 (1H relative int., v_{1/2}=450 Hz), 77.9 (1H, v_{1/2}=50 Hz), 53.1 (2H, v_{1/2}=480 Hz), 46.8 (2H, v_{1/2}=450 Hz), 39.4 (2H and 1H overlap, broad and sharp resonances), 21.8 and 19.5 (2H each, broad and overlapping), 13.0 (1H, v_{1/2}=400 Hz), −2.0 (1H, v_{1/2}=350 Hz), −4.2 (1H, v_{1/2}=350 Hz), −69e.8 (2H, v_{1/2}=450 Hz), −8.6 (1H, v_{1/2}=40 Hz), −28.7 (1H, v_{1/2}=50 Hz), −52.7 (1H, v_{1/2}=450 Hz), −72.8 (v_{1/2}=500 Hz). NMR (360 MHz, dmso-d₆, 373 K): δ 18.49 (6H, br s, 4′- or 5′-pzH, v_{1/2}=85 Hz), 17.00 (6H, br s, 3,5-pyH, v_{1/2}=65 Hz), 7.88 (6H, br s, 4′- or 5′-pzH, v_{1/2}=80 Hz), 4.18 (3H, br s, 4-pyH, v_{1/2}=30 Hz), −4.25 (2H, br s, BH, v_{1/2}=320 Hz). CV (0.1 M NBuⁿ₄⁺PF₆⁻, dry CH₃CN, 250 mV s⁻¹): E_{1/2 (ox)}=+1.1 V (vs. Fc/Fc⁺); Co(NO₃)₂ under the same conditions: E_{1/2 (ox)}=+1.8 V (vs. Fc/Fc⁺). μ_{eff}=4.7 BM (Evans' method). A mononuclear 1:1 complex [9a]of cage [4a] with Co²⁺ has been characterized by X-ray crystallography. An X-ray crystal structure of [9a] is shown in FIG. 5. A Co(OH₂)₃²⁺ unit is bound up in the tris(pyrazolyl) cap.

Ni Cage [4a]=Metal Complex [9b]:

Pale green powder. Yield: 99% (based on formula). Mp. dec. 310° C. (turns yellow at 250° C.). ¹H NMR (300 MHz, dmso-d₆, 298 K): δ 82.4 (1H relative int., v_{1/2}=90 Hz), 68.8 (3H, v_{1/2}=800 Hz), 62.8 (1H, v_{1/2}=500 Hz), 57.0 (1H, v_{1/2}=80 Hz), 37.5, 35.5 (4H total, overlapping, v_{1/2}=1000 Hz), 32.2 (1H, v_{1/2}=80 Hz), 17.1 (1H, v_{1/2}=40 Hz), 10.5 (5H, v_{1/2}=350 Hz), 6.5 (2H, v_{1/2}=120 Hz), −1.6 (1H, v_{1/2}=110 Hz), −10.9 (1H, v_{1/2}=1000 Hz). NMR (360 MHz, dmso-d₆, 373 K): δ 66.98 (6H, br s, 4′- or 5′-pzH, v_{1/2}=100 Hz), 47.34 (6H, br s, 3,5-pyH, v_{1/2}=100 Hz), 27.24 (6H, br s, 4′- or 5′-pzH, v_{1/2}=45 Hz), 15.23 (3H, br s, 4-pyH, v_{1/2}=40 Hz), −0.42 (2H, br s, BH, v_{1/2}=260 Hz). CV (0.1 M NBuⁿ₄⁺PF₆⁻, dry CH₃CN, 100 mV s⁻¹): E_{1/2 (ox)}=+1.4 V (vs. Fc/Fc⁺). μ_{eff}=3.2 BM (Evans' method).

Cu cage [4a]=metal complex [9c]:

A blue-green powder was obtained from reaction of Cu(NO₃)₂(H₂O)_{2.5} with [4a] in a 1:1 ratio. The ¹H NMR spectrum of this product is very similar to that of [11c′], obtained as one product in the reaction of Cu(O₃SCF₃)₂ with [4a] in 3:1 ratio. The magnetic susceptibility data suggests that this instead could be [Cu₂ cage [4a] (μ-OH)]⁺NO₃⁻, (observed μ_{eff}=2.6 BM (Evans' method) matches the expected spin-only value for two Cu²⁺ ions of 2.45 BM much better than that for a single Cu²⁺ ion of 1.73 BM). Until further data is obtained, however, this is listed as the mono Cu²⁺ cage 9c. Yield: 71% (based on formula). Mp. dec. 305° C. (turns yellow-green at 250° C.). ¹H NMR (300 MHz, dmso-d₆, 298 K): δ 64.22 (4H, br s, 4′- or 5′-pzH, v_{1/2}=60 Hz), 49.11 (4H, br s, 4′- or 5′-pzH, v_{1/2}=30 Hz), 26.55 (4H, br s, 3,5-pyH, v_{1/2}=21 Hz), 12.39 (2H, br s, 4-pyH, v_{1/2}=16 Hz), 8.05 (2H, br d, ³J_{HH}=7.0 Hz, non-coord. 3,5-pyH), 7.93 (1H, br t, ³J_{HH}=7.0 Hz, non-coord. 4-pyH), 7.78 (2H, br s, non-coord. 5′-pzH), 6.50 (2H, br s, non-coord. 4′-pzH), -0.42 (2H, br s, BH, v_{1/2}=70 Hz). CV (0.1 M NBuⁿ₄⁺PF₆⁻, dry CH₃CN, 50 mV s⁻¹): E_{1/2 (red)}=−1.2 V (vs. Fc/Fc⁺); Cu(NO₃)₂ under the same conditions: E_{1/2 (red)}=−0.8 V (vs. Fc/Fc⁺).

Zn Cage [4a]=Metal Complex [9d]:

White powder. Yield: 95% (based on formula). Mp.>330° C. ¹H NMR (500 MHz, dmso-d₆): δ 8.08 (6H, 2, ³J_{HH}=2.2 Hz, 5′-pzH), 8.02 (3H, t, ³J_{HH}=7.4 Hz, 4-pyH), 7.93 (6H, d, ³J_{HH}=7.4 Hz, 3,5-pyH), 7.04 (6H, d, ³J_{HH}=2.2 Hz, 4′-pzH), 4.83 (2H, br s, BH); ¹³C{¹H} (125 MHz, dmso-d₆): δ 151.21 (2,6-pyC), 150.62 (3′-pzC), 138.84 (4-pyCH), 138.53 (5′-pzCHl), 121.61 (3,5-pyCH), 105.22 (4′-pzCHl); ¹¹B NMR (160.51 MHz, dmso-d₆): δ −3.0 (v br s). CV (0.1 M NBuⁿ₄⁺PF⁻, dry CH₃CN, 100 mV s⁻¹): no oxidation or reductions within the solvent window (−3 to +2 V).

Cd Cage [4a]=Metal Complex [9e]:

White powder. Yield: 45% (based on isolated solid matching this formula). Mp. dec. 280° C. (turns yellow-brown). ¹H NMR (300 MHz, dmso-d₆): δ 7.90 (6H, d, ³J_{HH}=2.1 Hz, 5′-pzH), 7.88 (3H, t, ³J_{HH}=7.8 Hz, 4-pyH), 7.73 (6H, d, ³J_{HH}=7.8 Hz, 3,5-pyH), 6.87 (6H, d, ³J_{HH}=2.1 Hz, 4′-pzH), 4.80 (2H, br s, BH); ¹³C{¹H} (75 MHz, dmso-d₆): δ 150.77 (2,6-pyC), 150.66 (3′-pzC), 138.77 (4-pyCH), 138.12 (5′-pzCH), 119.44 (3,5-pyCH), 103.34 (4′-pzCH); ¹¹B NMR (115.6 MHz, dmso-d₆): δ 0.3 (v br d, J=44 Hz).

Mg Cage [4a]·3H₂O=Metal Complex [9f]:

White powder. Yield: 76% (based on this formula). Mp.>330° C. ¹H NMR (500 MHz, dmso-d₆:acetone-d₆ 5:1): δ 8.446 (6H, br s, OH₂), 8.019 (6H, br s, 5′-pzH), 7.967 (3H, t, ³J_{HH}=8.0 Hz, 4-pyH), 7.821 (6H, d, ³J_{HH}=8.0 Hz, 3,5-pyH), 6.983 (6H, br s, 4′-pzH), 4.88 (2H, br s, BH); ¹³C{¹H} (125 MHz, dmso-d₆:acetone-d₆ 5:1): δ 151.57 (2,6-pyC), 150.26 (3′-pzC), 138.95 (4-pyCH), 137.67 (5′-pzCHl), 121.10 (3,5-pyCH), 104.63 (4′-pzCHl); ¹¹B NMR (160.51 MHz, dmso-d₆:acetone-d₆ 5:1): δ −1.6 (v br s); +ESI MS (150V cone voltage, i-PrOH): [Mg Cage [4a]⁺H⁺]⁺, observed: 674 (8), 675 (47), 676 (100), 677 (56), 678 (24), 679 (12); calculated: 674 (5), 675 (41), 676 (100), 677 (53), 678 (25), 679 (7).

Ca Cage [4a]=Metal Complex [9g]:

White powder. Yield: 49% Mp.>330° C. ¹H NMR (500 MHz, 1:1 acetone-d₆:dmso-d₆): δ 7.918 (6H, br s, 5′-pzH), 7.877 (3H, br t, ³J_{HH}=7.5 Hz, 4-pyH), 7.692 (6H, br d, ³J_{HH}=7.5 Hz, 3,5-pyH), 6.841 (6H, br s, 4′-pzH), 4.91 (2H, br m, BH); ¹³C{¹H} (125 MHz, 1:1 acetone-d₆:dmso-d₆): δ 151.59 (2,6-pyC), 151.35 (3′-pzC), 138.51 (4-pyCH), 138.16 (5′-pzCHl), 118.22 (3,5-pyCH), 102.58 (4′-pzCHl); ¹¹B NMR (160.51 MHz, 1:1 acetone-d₆:dmso-d₆): δ −1.34 (br s); +ESI MS (175V cone voltage, i-PrOH): [Ca cage [4a]+H⁺]⁺, observed: 689 (15), 690 (38), 691 (54), 692 (100), 693 (36), 694 (10), 695 (3); calculated: 690 (5), 691 (43), 692 (100), 693 (38), 694 (8), 695 (2). Loss of H₂ from [[9g]⁺H]⁺ results in a larger than expected peak at 690; this is often observed in positive ESI for [cage+H]⁺ ions. Note that the match between observed and calculated intensities is excellent for masses greater than the molecular ion.

Sr Cage [4a]=Metal Complex [9h]:

White powder. Yield: 72%. Mp.>330° C. ¹H{¹¹B} NMR (500 MHz, dmso-d₆:acetone-d₆ 1:1): δ 7.890 (6H, d, ³J_{HH}=2.5 Hz, 5′-pzH), 7.840 (3H, t, ³J_{HH}=7.5 Hz, 4-pyH), 7.699 (6H, d, ³J_{HH}=7.5 Hz, 3,5-pyH), 6.857 (6H, d, ³J_{HH}=2.5 Hz, 4′-pzH), 4.84 (2H, br s, BH); ¹³C{¹H} (125 MHz, dmso-d₆:acetone-d₆ 1:1): δ 152.00 (2,6-pyC), 151.36 (3′-pzC), 138.21 (4-pyCH), 137.42 (5′-pzCHl), 118.63 (3,5-pyCH), 102.79 (4′-pzCHl); ¹¹B NMR (160.51 MHz, dmso-d₆:acetone-d₆ 1:1): δ −1.67 (br s); +ESI MS (175V cone voltage, i-PrOH): [Sr cage [4a]⁺H⁺]⁺, observed: 734 (3), 735 (6), 736 (20), 737 (51), 738 (100), 739 (76), 740 (93), 741 (34), 742 (8), 743 (3); calculated: 737 (6), 738 (20), 739 (54), 740 (100), 741 (37), 742 (7). Loss of H₂ from [[9h]⁺H]⁺ results in a larger than expected peak at masses below the molecular ion; this is often observed in positive ESI for [cage+H]⁺ ions. This is essentially caused by a shift of the isotopic pattern 2 units to lower mass. Although the [[9h]⁺K]⁺ is considerably weaker (20% the size of the molecular ion), it does not show loss of H₂ and shows an excellent match between the observed and calculated ion intensities (those with observed and calculated intensities >3% shown). [[9h]⁺K]⁺ observed: 775 (8), 776 (21), 777 (54), 778 (100), 779 (40), 780 (15), 781 (5); calculated: 775 (5), 776 (20), 777 (53), 778 (100), 779 (40), 780 (14), 781 (3).

Ba Cage [4a]=Metal Complex [9i]:

White powder. Yield: 86%. Mp.>330° C. ¹H NMR (500 MHz, dmso-d₆): δ 7.863 (6H, d, ³J_{HH}=2.5 Hz, 5′-pzH), 7.818 (3H, t, ³J_{HH}=8.0 Hz, 4-pyH), 7.714 (6H, d, ³J_{HH}=8.0 Hz, 3,5-pyH), 6.873 (6H, d, ³J_{HH}=2.5 Hz, 4′-pzH), 4.73 (2H, br s, BH); ¹³C{¹H} (125 MHz, dmso-d₆): δ 152.07 (2,6-pyC), 151.56 (3′-pzC), 137.90 (4-pyCH), 136.73 (5′-pzCH), 118.85 (3,5-pyCH), 102.91 (4′-pzCHl); ¹¹B NMR (160.51 MHz, dmso-d₆): δ −1.9 (v br s); +ESI MS (100V cone voltage, i-PrOH): [Ba cage [4a]⁺H⁺]⁺, observed: 785 (4), 786 (9), 787 (17), 788 (24), 789 (66), 790 (100), 791 (38), 792 (8); calculated: 785 (2), 786 (7), 787 (15), 788 (25), 789 (59), 790 (100), 791 (36), 792 (7).

Pb Cage [4a]=Metal Complex [9j]:

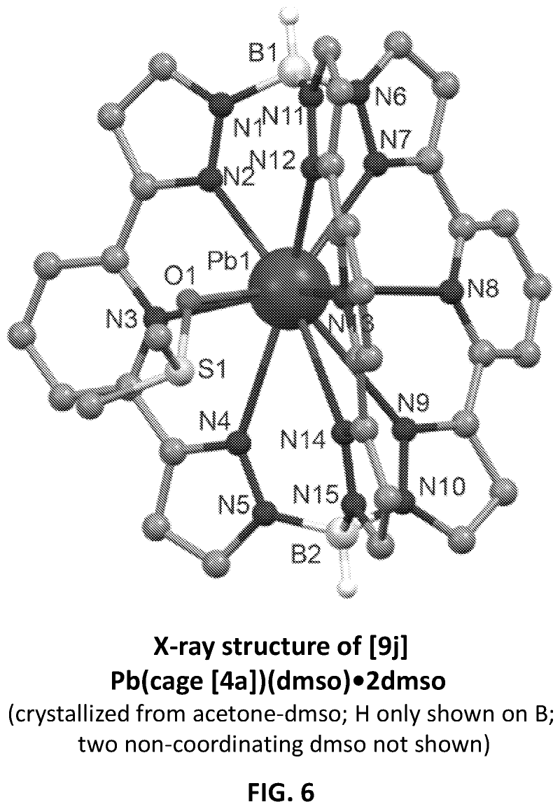

White powder. Yield: 45%. Mp.>330° C. ¹H{¹¹B} NMR (500 MHz, dmso-d₆:acetone-d₆ 1:1): δ 7.905 (6H, d, ³J_{HH}=2.0 Hz, 5′-pzH), 7.877 (3H, t, ³J_{HH}=8.0 Hz, 4-pyH), 7.755 (6H, d, ³J_{HH}=8.0 Hz, 3,5-pyH), 6.904 (6H, d, ³J_{HH}=2.0 Hz, 4′-pzH), 4.86 (2H, br s, BH); ¹³C{¹H} (125 MHz, dmso-d₆:acetone-d₆ 1:1): δ 151.94 (2,6-pyC), 151.36 (3′-pzC), 138.33 (4-pyCH), 137.63 (5′-pzCHl), 118.97 (3,5-pyCH), 103.15 (4′-pzCHl); ¹¹B NMR (160.51 MHz, dmso-d₆:acetone-d₆ 1:1): δ −1.77 (br s); +ESI MS (200V cone voltage, MeOH): [Pb cage [4a]⁺H⁺]⁺, observed: 855 (1), 856 (4), 857 (20), 858 (60), 859 (86), 860 (100), 861 (38), 862 (8); calculated: 855 (1), 856 (4), 857 (19), 858 (58), 859 (86), 860 (100), 861 (35), 862 (7). A mononuclear 1:1 complex [9j] of cage [4a] with Pb²⁺ has been characterized by X-ray crystallography. An X-ray crystal structure of [9j] is shown in FIG. 6. This structure shows 10-coordinate Pb²⁺ with an oxygen-bonded dmso molecule. The latter is unusual for Pb, as it usually binds dmso through the sulfur.

[Al cage [4a]]⁺ NO₃⁻=metal complex [10a]·10H₂O:

White powder. Yield: 58% (based on this formula). Mp. dec.>300° C. ¹H{¹¹B} NMR (500 MHz, dmso-d₆, 298K): δ 8.05 (6H, br s, 5′-pzH), 7.82 (3H, br m, 4-pyH), 7.76 (6H, br m, 3,5-pyH), 6.95 (6H, br s, 4′-pzH), 4.82 (2H, br s, BH), 4.16 (20H, br s, H₂O); ¹¹B NMR (160.51 MHz, dmso-d₆, 298K): δ −1.9 (v br s). ¹H{¹¹B} NMR (500 MHz, dmso-d₆, 373K): δ 7.869 (3H, t, ³J_{HH}=7.5 Hz, 4-pyH), 7.784 (6H, d, ³J_{HH}=7.5 Hz, 3,5-pyH), 7.68 (6H, br s, 5′-pzH), 6.98 (6H, br s, 4′-pzH), 4.86 (2H, br s, BH), 3.53 (20H, br s, H₂O). The sample decomposes rapidly above 75° C. with formation of borate species as evident from the resonance at +19.98 ppm in the ¹¹B NMR. Elem. analysis calcd. for [10a]·3 H₂O (closest fit to hydration state after vacuum at 50° C. overnight) (C₃₃H₂₉N₁₆B₂O₆Al): C, 49.90; H, 3.68; N, 28.22%. Found: C, 49.78; H, 3.81; N, 27.97%.

[Sc Cage [4a]]⁺ NO₃⁻=Metal Complex [10b]·H₂O·Acetone:

Very pale yellow powder. Yield: 76% (based on this formula). Mp. dec.>300° C. ¹H NMR (500 MHz, dmso-d₆): δ 7.806 (6H, d, ³J_{HH}=2.2 Hz, 5′-pzH), 7.765 (3H, t, ³J_{HH}=7.8 Hz, 4-pyH), 7.585 (6H, d, ³J_{HH}=7.8 Hz, 3,5-pyH), 6.743 (6H, d, ³J_{HH}=2.2 Hz, 4′-pzH), 4.80 (2H, br s, BH), 4.16 (2H, br s, H₂O), 2.09 (6H, s, O═C(CH₃)₂); ¹³C{¹H} (125 MHz, dmso-d₆): δ 206.53 (C═O), 151.69 (2,6-pyC), 150.84 (3′-pzC), 137.94 (4-pyCH), 136.52 (5′-pzCH), 118.39 (3,5-pyCH), 102.67 (4′-pzCH), 30.70 (O═C(CH₃)₂); ¹¹B NMR (160.51 MHz, dmso-d₆): δ −1.6 (v br s); ⁴⁵Sc NMR (121.54 MHz, dmso-d₆): δ 48.2; +ESI MS (125V cone voltage, THF): [Sc cage [4a] (THF)]⁺, observed: 766 (8), 767 (44), 768 (100), 769 (44), 770 (26); calculated: 766 (5), 767 (42), 768 (100), 769 (42), 770 (9).

[Y Cage [4a]]⁺NO₃⁻=Metal Complex [10c]:

White powder from water, washed extensively with water and finally D₂O before drying. Yield: 65% (based on formula). Mp. dec. 310° C. ¹H NMR (300 MHz, dmso-d₆): δ 8.05 (9H, d and t overlapping, 5′-pzH and, 4-pyH), 7.98 (6H, d, ³J_{HH}=7.2 Hz, 3,5-pyH), 7.05 (6H, d, ³J_{HH}=2.0 Hz, 4′-pzH), 4.86 (2H, br s, BH); ¹³C{¹H} (75 MHz, dmso-d₆): δ 151.32 (2,6-pyC), 150.79 (3′-pzC), 140.4 (br, 5′-pzH or 4-pyCH), 139.48 (4-pyH or 5′-pzCHl), 118.89 (3,5-pyCH), 103.89 (4′-pzCHl); ¹¹B NMR (115.6 MHz, dmso-d₆): δ +0.9 (v br d, J=77 Hz); +ESI MS (75V cone voltage, dmso-d₆): [Y cage [4a] (dmso-d₆)(H₂O)]⁺, observed: 840 (6), 841 (42), 842 (100), 843 (38), 844 (12); calculated: 840 (5), 841 (42), 842 (100), 843 (42), 844 (13).

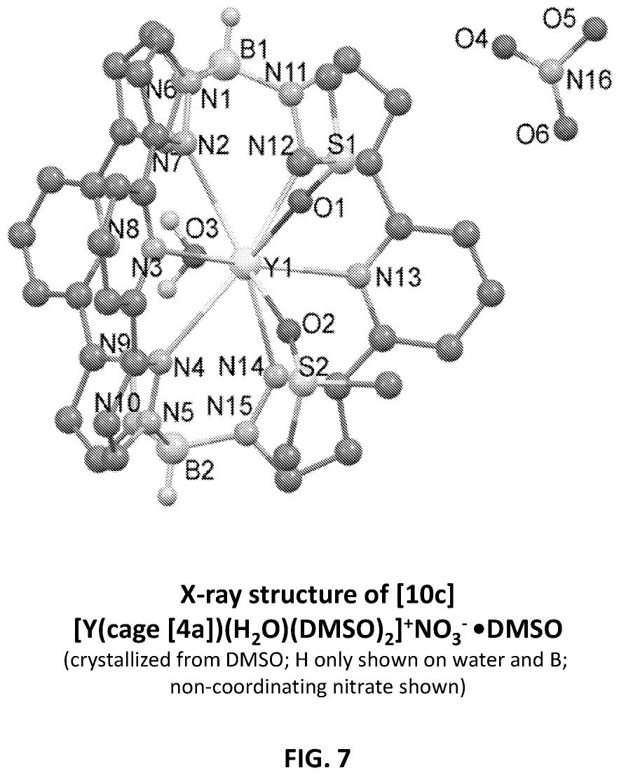

A mononuclear 1:1 complex [10c] of cage [4a] with Y³⁺ has been characterized by X-ray crystallography. An X-ray crystal structure of [[Y cage [4a] (H₂O)(DMSO)₂]]⁺NO₃⁻·DMSO is shown in FIG. 7. The yttrium is coordinated by a water molecule, the oxygen atoms of two DMSO molecules and two pyridine and four pyrazole groups from the cage ligand (as can be seen in FIG. 7), yielding a 9-coordinate environment. Viewed along the water-yttrium bond the coordination environment could be described as a distorted hexagonal bipyramid, with the water occupying one apical site and the two oxygen atoms of DMSO occupying the other, with the coordinating nitrogen atoms in the equatorial plane. The ‘equatorial’ plane is, however, ruffled when viewed through the edge of the plane. It could also be described as a highly distorted, face-capped trigonal prism. The coordinating water forms hydrogen bonds to the non-coordinating pyrazole nitrogen atoms within the cage. One of the coordinating DMSO molecules and the nitrate anion are disordered, and were modeled with 50% occupancy atoms.

[La Cage [4a]]⁺NO₃⁻·Acetone=Metal Complex [10d]:

White powder. Yield: 55% (based on formula). Mp. dec. 310° C. (turns orange-red but does not melt to 340° C.). ¹H NMR (500 MHz, dmso-d₆): δ 7.97 (6H, s, 5′-pzH), 7.93 (3H, t, ³J_{HH}=7.5 Hz, 4-pyH), 7.83 (6H, d, ³J_{HH}=7.5 Hz, 3,5-pyH), 7.01 (6H, s, 4′-pzH), 4.79 (2H, br s, BH), 2.09 (6H, s, O═C(CH₃)₂); ¹³C{¹H} (125 MHz, dmso-d₆): δ 206.67 (C═O), 151.95 (2,6-pyC), 150.18 (3′-pzC), 138.0 (v. br, 4-pyCH), 137.3 (v. br, 5′-pzCH), 119.08 (3,5-pyCH), 103.37 (4′-pzCH), 30.76 (O═C(CH₃)₂); ¹¹B NMR (160.51 MHz, dmso-d₆): δ −2.8 (v br s); +ESI MS (150V cone voltage, MeOH): [La cage [4a] (CH₃₀H)]⁺, observed: 820 (7), 821 (45), 822 (100), 823 (37), 824 (8); calculated: 820 (7), 821 (43), 822 (100), 823 (39), 824 (8). Elem. analysis calcd. for [10d] (after vacuum at 100° C. overnight) (C₃₃H₂₃N₁₆B₂O₃La): C, 46.51; H, 2.72; N, 26.30%. Found: C, 46.44; H, 2.80; N, 26.15%.

[Ce Cage [4a]]⁺NO₃⁻·2(H₂O)=Metal Complex [10e]:

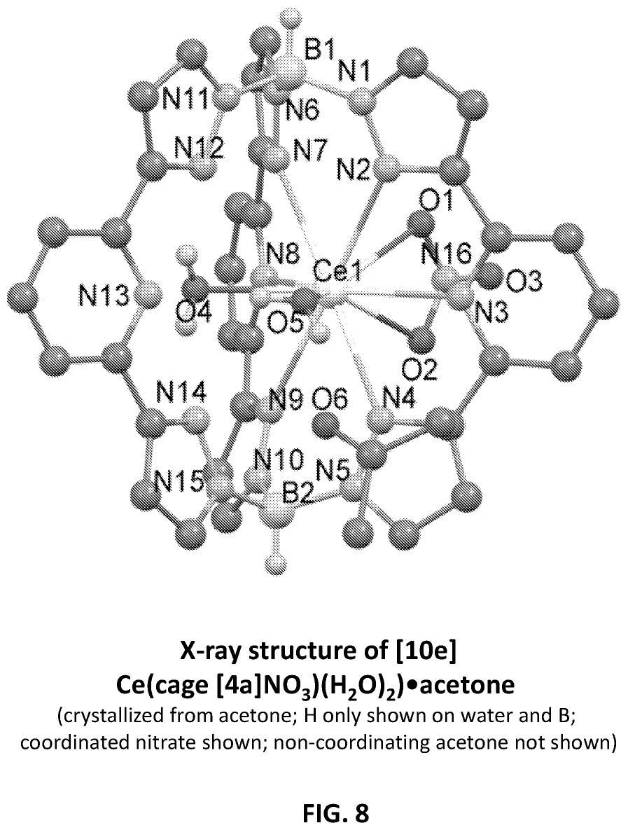

Slightly off-white powder. Yield: 97% (based on this formula). Mp. dec.>300° C. ¹H NMR (300 MHz, dmso-d₆, 298 K): δ 12.28 (2H, br s, v_{1/2}=18 Hz), 11.55 (1H, br s, v_{1/2}=24 Hz), 8.93 (2H, br s, v_{1/2}=15 Hz), 8.05 (2H, br s, v_{1/2}=30 Hz), 7.35 (2H, br s, v_{1/2}=10 Hz), 7.30 (1H, br s, v_{1/2}=16 Hz, overlaps previous signal), 7.18 (2H, br s, v_{1/2}=8 Hz), 7.01 (2H, br s, v_{1/2}=8 Hz), 6.23 (2H, br s, v_{1/2}=18 Hz), 6.01 (1H, br s, v_{1/2}=24 Hz), 5.88 (2H, br s, v_{1/2}=18 Hz), 5.70 (2H, br s, v_{1/2}=18 Hz), 4.85 (1H, br s, v_{1/2}=20 Hz, BH), 4.69 (1H, br s, v_{1/2}=20 Hz, BH), -1.10 (4H, br s, v_{1/2}=100 Hz, likely coordinated water); +ESI MS (75V cone voltage, dmso-d₆): [Ce cage [4a] (dmso-d₆)₂]⁺, observed: 957 (6), 958 (44), 959 (100), 960 (54), 961 (32), 962 (10); calculated: 957 (5), 958 (42), 959 (100), 960 (51), 961 (32), 962 (11). Elem. analysis calcd. for [10e]·2 H₂O (after vacuum at 100° C. overnight) (C₃₃H₂₇N₁₆B₂O₅Ce): C, 44.56; H, 3.06; N, 25.20%. Found: C, 44.43; H, 3.10; N, 24.85%. A mononuclear 1:1 complex [10e] of cage [4a] with Ce³⁺ has been characterized by X-ray crystallography. An X-ray crystal structure of [Ce cage [4a] (κ²-O,O—NO₃)(H₂O)₂]·acetone is shown in FIG. 8. There is one molecule of the cerium complex and an associated molecule of acetone of crystallization in the asymmetric unit of the primitive, centrosymmetric monoclinic space group P2₁/c. The cerium is coordinated in a 9-coordinate fashion by two water molecules, a κ² nitrate (counting as occupying one coordination site for the purposes of this discussion) and the pyrazole nitrogen atoms from four of the six pyrazole rings, and two pyridine nitrogen atoms out of the three present on cage ligand [4a]. The coordinated water molecules form hydrogen bonds to nearby nitrogen atoms of the unbound pyrazole and pyridine moieties of the cage ligand, and to the oxygen atom of acetone.

[Nd Cage [4a]]⁺NO₃⁻⁼ Metal Complex [10f]:

Slightly off-white powder. Yield: 92% (based on this formula). Mp. dec.>300° C. ¹H NMR spectrum too broad to observe. +ESI MS (75V cone voltage, MeOH): [Nd cage [4a]]⁺, observed: 792 (50), 793 (74), 794 (100), 795 (92), 796 (86), 797 (61), 798 (28), 799 (24), 800 (19), 801 (15), 802 (11); calculated: 792 (35), 793 (96), 794 (95), 795 (100), 796 (74), 797 (65), 798 (28), 799 (21), 800 (14), 801 (17), 802 (6). Elem. analysis calcd. for [10f]·H₂O (after vacuum at 100° C. overnight) (C₃₃H₂₅N₁₆B₂O₄Nd): C, 45.27; H, 2.88; N, 25.60%. Found: C, 44.98; H, 3.00; N, 25.45%.

[Sm Cage [4a]]⁺ NO₃⁻=Metal Complex [10g]:

White powder. Yield: 70% (based on this formula). Mp. dec.>300° C.; ¹H NMR (300 MHz, dmso-d₆, 293 K): δ 7.929 (3H, br t, ³J_{HH}=8.0 Hz, 4-pyH), 7.722 (6H, br s, 5′-pzH), 7.637 (6H, br d, ³J_{HH}=8.0 Hz, 3,5-pyH), 6.773 (6H, br s, 4′-pzH), BH resonance obscured by residual water in dmso-d₆ at 4.7 ppm; +ESI MS (125V cone voltage, MeOH): [Sm cage [4a] (CH₃₀H)]⁺, observed: 826 (7), 827 (13), 828 (8), 829 (40), 830 (90), 831 (93), 832 (95), 833 (60), 834 (65), 835 (100), 836 (74), 837 (90), 838 (36), 839 (9); calculated: 826 (5), 827 (11), 828 (7), 829 (26), 830 (75), 831 (85), 832 (82), 833 (55), 834 (55), 835 (100), 836 (72), 837 (90), 838 (33), 839 (7). Elem. analysis calcd. for [10g]·H₂O (after vacuum at 100° C. overnight) (C₃₃H₂₅N₁₆B₂O₄Sm): C, 44.96; H, 2.86; N, 25.42%. Found: C, 44.77; H, 2.98; N, 25.27%.

[Gd Cage [4a]]⁺ NO₃⁻=Metal Complex [10h]:

White powder. Yield: 80% (based on this formula). Mp. dec.>300° C. ¹H NMR spectrum too broad to observe. +ESI MS (175V cone voltage, MeOH): [Gd cage [4a]]⁺, observed: 804 (9), 805 (26), 806 (75), 807 (99), 808 (98), 809 (100), 810 (60), 811 (65), 812 (35), 813 (10); calculated: 804 (5), 805 (27), 806 (72), 807 (97), 808 (100), 809 (95), 810 (57), 811 (68), 812 (24), 813 (5). Elem. analysis calcd. for [10h]·H₂O (after vacuum at 100° C. overnight) (C₃₃H₂₅N₁₆B₂O₄Gd): C, 44.61; H, 2.84; N, 25.22%. Found: C, 44.30; H, 3.02; N, 25.01%.

[Tb Cage [4a]]⁺NO₃⁻·Acetone=Metal Complex [10i]:

White powder. Yield: 78% (based on formula). Mp. dec.>300° C. ¹H NMR spectrum too broad to observe. +ESI MS (125V cone voltage, MeOH): [Tb cage [4a] (CH₃₀H)]⁺, observed: 840 (6), 841 (43), 842 (100), 843 (34), 844 (7); calculated: 840 (5), 841 (43), 842 (100), 843 (39), 844 (8). Elem. analysis calcd. for [10i]·H₂O (after vacuum at 100° C. overnight) (C₃₃H₂₅N₁₆B₂O₄Tb): C, 44.52; H, 2.83; N, 25.18%. Found: C, 44.33; H, 2.96; N, 24.88%.

[Tm Cage [4a]]⁺ NO₃⁻=Metal Complex [10j]:

White powder. Yield: 60% (based on this formula). ¹H NMR (300 MHz, 3:1 acetone-d₆:dmso-d₆): δ 48.3 (6H, v_{1/2}=420 Hz), 42.3 and 41.6 overlapping (12H, v_{1/2}=630 Hz), 36.4 (3H, v_{1/2}=180 Hz), 25.3 (2H, v_{1/2}=360 Hz); +ESI MS (75V cone voltage, MeOH): [Tm cage [4a] (d₆-dmso)(H₂O)]⁺, observed: 920 (8), 921 (46), 922 (100), 923 (38), 924 (16), 925 (6); calculated: 920 (5), 921 (43), 922 (100), 923 (42), 924 (13), 925 (3). Elem. analysis calcd. for [10j]·H₂O (after vacuum at 100° C. overnight) (C₃₃H₂₅N₁₆B₂O₄Tm): C, 44.03; H, 2.80; N, 24.90%. Found: C, 43.87; H, 2.89; N, 24.67%.

[Yb Cage [4a]]⁺ NO₃⁻=Metal Complex [10k]:

D White powder. Yield: 90% (based on this formula). ¹H NMR (300 MHz, 3:1 acetone-d₆:dmso-d₆): δ 32.6 (6H, v_{1/2}=270 Hz), 28.4 (6H, v_{1/2}=240 Hz), 23.7 (2H, v_{1/2}=240 Hz), 19.2 (6H, v_{1/2}=270 Hz), 10.6 (3H, v_{1/2}=210 Hz); +ESI MS (75V cone voltage, MeOH): [Yb cage [4a] (d₆-dmso)(H₂O)]⁺, observed: 922 (5), 923 (32), 924 (63), 925 (83), 926 (97), 927 (100), 928 (53), 929 (40), 930 (15); calculated: 922 (5), 923 (24), 924 (60), 925 (86), 926 (96), 927 (100), 928 (51), 929 (41), 930 (15). Elem. analysis calcd. for [10k]·H₂O (after vacuum at 100° C. overnight) (C₃₃H₂₅N₁₆B₂O₄Yb): C, 43.83; H, 2.79; N, 24.78%. Found: C, 43.60; H, 2.88; N, 24.51%.

[Lu Cage [4a]]⁺NO₃⁻=Metal Complex [10l]:

White powder. Yield: 91% (based on this formula). Mp.>300° C. ¹H NMR (500 MHz, 3:1 dmso-d₆:acetone-d₆): δ 8.035 (6H, br d, ³J_{HH}=2.5 Hz, 5′-pzH), 7.894 (9H, v br s, 3,5- and 4-pyH), 7.032 (6H, br s, 4′-pzH), 4.93 (2H, br m, BH); ¹³C{¹H} (125 MHz, 3:1 dmso-d₆:acetone-d₆): δ 151.47 (2,6-pyC), 151.26 (3′-pzC), 139.34 (4-pyCH), 138.18 (5′-pzCHl), 118.90 (3,5-pyCH), 103.23 (4′-pzCHl); ¹¹B{¹H} (160.51 MHz, 3:1 dmso-d₆:acetone-d₆): δ −2.24; +ESI MS (150V cone voltage, IPA): [Lu cage [4a]]⁺, observed: 824 (8), 825 (43), 826 (100), 827 (40), 828 (12); calculated: 824 (5), 825 (43), 826 (100), 827 (40), 828 (8) and [Lu cage [4a] (i-PrOH)]⁺, observed: 884 (6), 885 (41), 886 (100), 887 (45), 888 (11); calculated: 884 (5), 885 (42), 886 (100), 887 (43), 888 (10) with the relative intensity of the IPA adduct:base-free cage 100:96. Elem. analysis calcd. for [101]·H₂O (after vacuum at 100° C. overnight) (C₃₃H₂₅N₁₆B₂O₄Lu): C, 43.73; H, 2.78; N, 24.73%. Found: C, 43.61; H, 2.91; N, 24.63%.

[Ga Cage [4a]]⁺ NO₃=Metal Complex [10m]:

White powder. Yield: 55% (based on this formula). Mp. dec.>300° C. ¹H{¹¹B} NMR (500 MHz, 3:1 acetone-d₆:dmso-d₆, 293K): δ 8.037 (4H, br s, 5′-pzH), 7.934 (2H, d, ³J_{HH}=2.5 Hz, 5′pzH non-coordinating), 7.885 (4H, d, ³J_{HH}=8.0 Hz, 3,5-pyH), 7.723 (2H, d, ³J_{HH}=8.0 Hz, 3,5-pyH non-coordinating), 7.778 (3H, br s, 4-pyH), 6.947 (4H, br s, 4′-pzH), 6.876 (2H, br s, 4′-pzH non-coordinating), 4.90 (2H, br s, BH); ¹¹B NMR (160.51 MHz, 3:1 acetone-d₆:dmso-d₆, 293K): δ −1.9 (v br s). Positive ESI MS gave predominantly a fragment corresponding to [Ga(C₁₁H₈N5)₂B−H]⁺ at a cone voltage of 125V from IPA solvent. Increasing the cone voltage to 200 V gave a weak intensity ion for [[Ga cage [4a] (H₂O)]⁺. +ESI MS (125V cone voltage, IPA): [Ga(C₁₁H₈N₅)₂B−H]⁺, observed: 500 (28), 501 (100), 502 (46), 503 (70), 504 (19), 505 (5); calculated: 500 (23), 501 (100), 502 (42), 503 (70), 504 (18), 505 (3). +ESI MS (200V cone voltage, IPA): [[Ga cage [4a] (H₂O)]⁺, observed: 736 (20), 737 (63), 738 (100), 739 (57), 740 (47), 741 (29), 742 (18); calculated: 736 (5), 737 (42), 738 (100), 739 (64), 740 (72), 741 (25), 742 (5). The pattern corresponding to [[Ga cage [4a] (H₂O)]⁺ is not a perfect match but this is likely due to the relatively low signal intensity and the loss of H₂ from this ion which is to be expected at the high cone voltage required to observe it. Elem. analysis calcd. for [10m] (after vacuum at 100° C. overnight) (C₃₃H₂₅N₁₁B₂O₄Ga): C, 50.62; H, 2.96; N, 28.62%. Found: C, 50.23; H, 2.87; N, 28.29%.

[Hf Cage [4a]]²⁺ (CF₃SO₃⁻)₂(H₂O)ₓ (x~8)=Metal Complex [10n]:

Prepared using excess Hf(CF₃SO₃)₄ dissolved in acetone with ca. 10% water (0.1 g in 5 mL acetone) and 0.100 g cage [4a]. A white solid precipitated within 15 minutes and the solution was allowed to stir for 2 h. Filtration and washing the solid with diethyl ether followed by vacuum drying afforded 0.036 g of [10n] as a white powder. Yield: 41% (based on this formula). Mp.>300° C. ¹H{¹¹B} NMR (500 MHz, 1:1 acetone-d₆:dmso-d₆): δ 8.283 (6H, br s, 5′-pzH), 8.070 (9H, br s, 3,5- and 4-pyH), 7.221 (6H, br s, 4′-pzH), 4.92 (2H, br s, BH); ¹³C{¹H} (125 MHz, 1:1 acetone-d₆:dmso-d₆): δ 148.95 (2,6-pyC), 147.77 (3′-pzC), 140.63 (4-pyCH), 139.56 (5′-pzCHl), 122.39 (3,5-pyCH), 105.73 (4′-pzCHl); ¹¹B (160.51 MHz, 1:1 acetone-d₆:dmso-d₆): δ −2.11.

[Co₂ Cage [4a]]⁴⁺ [NO₃⁻]₄(H₂O)ₓ=Metal Complex [11a]:

Deep mauve powder. Yield: 80% (based on formula, x=3). Mp. dec.>300° C. ¹H NMR (300 MHz, 3:1 acetone-d₆:dmso-d₆, 293 K): predominant broad series of peaks 6 79.40 (6H, br s, v_{1/2}=600 Hz), 52.03 (6H, br s, v_{1/2}=600 Hz), 24.80 (3H, br s, v_{1/2}=400 Hz), 20.88 (6H, br s, v_{1/2}=500 Hz), −24.75 (2H, br s, BH, vkk_{1/2}=900 Hz), a series of several smaller and mostly sharp resonances is also observed at 6 85.53, 76.23, 65.01, 60.53, 58.38, 55.77, 50.41, 43.29, 41.02, 39.08, 31.96, 8.46, -2.27, -12.46, -36.91, -55.11, -96.1. These resonances could represent minor impurities, partially complexed cage or variations in nitrate counterion coordination; +ESI MS (75V cone voltage, MeOH): [CO₂ cage [4a] (OH)]⁺, observed: 784 (5), 785 (46), 786 (100), 787 (36), 788 (7); calculated: 784 (5), 785 (43), 786 (100), 787 (38), 788 (8).

[Ni₂ Cage [4a] (OH)]⁺ [NO₃⁻]=Metal Complex [11b]:

Pale green powder. Yield: 72% (based on formula). Mp. dec.>300° C. ¹H NMR (300 MHz, 1:1 acetone-d₆:dmso-d₆, 293 K): δ 69.34 (6H, br s, 4′- or 5′-pzH, v_{1/2}=780 Hz), 36.05 (6H, br s, 3,5-pyH, v_{1/2}=550 Hz), 10.37 (6H, br s, 4′ or 5′-pzH, v_{1/2}=300 Hz), 6.62 (3H, br s, 4-pyH, v_{1/2}=120 Hz), −0.42 (2H, br s, BH, v_{1/2}=150 Hz); +ESI MS (75V cone voltage, MeOH): [N₁₂ cage [4a] (OH)(CH₃₀H)]⁺, observed: 814 (8), 815 (40), 816 (100), 817 (72), 818 (88), 819 (38), 820 (34), 821 (16), 822 (12); calculated: 814 (5), 815 (41), 816 (100), 817 (69), 818 (84), 819 (43), 820 (32), 821 (14), 822 (9); μ_{eff}=4.8 BM (Evans' method) is consistent with two non-interacting Ni²⁺ ions (predicted value based on typical Ni2+ moment is 4.1-5.5 BM).

[Cu₃ Cage [4a] (μ³-O)]2+ [NO₃⁻]₄₂=Metal Complex [11c]:

Blue powder. Yield: 85% (based on formula). Mp. dec.>300° C. ¹H NMR (300 MHz, dmso-d₆, 298 K): δ 43.82 (6H, br s, 4′- or 5′-pzH, v_{1/2}=36 Hz), 26.55 (6H, br d, 3,5-pyH, v_{1/2}=18 Hz), 21.70 (6H, br s, 4′ or 5′-pzH, v_{1/2}=12 Hz), 8.66 (3H, br t, 4-pyH), -3.08 (2H, br s, BH, v_{1/2}=80 Hz); +ESI MS (75V cone voltage, MeOH): [Cu₃ cage [4a] (O)]⁺, observed: 855 (35), 856 (70), 857 (66), 858 (100), 859 (60), 860 (50), 861 (20), 862 (10); calculated: 855 (30), 856 (74), 857 (66), 858 (100), 859 (53), 860 (49), 861 (19), 862 (9); μ_{eff}=3.2 BM (Evans' method) compared to an expected value of 3.0-3.6 BM for three non-interacting Cu²⁺ ions.

[Cu₂ Cage [4a] (μ²-OH)]⁺ [CF₃SO₃⁻]=Metal Complex [11c′]:

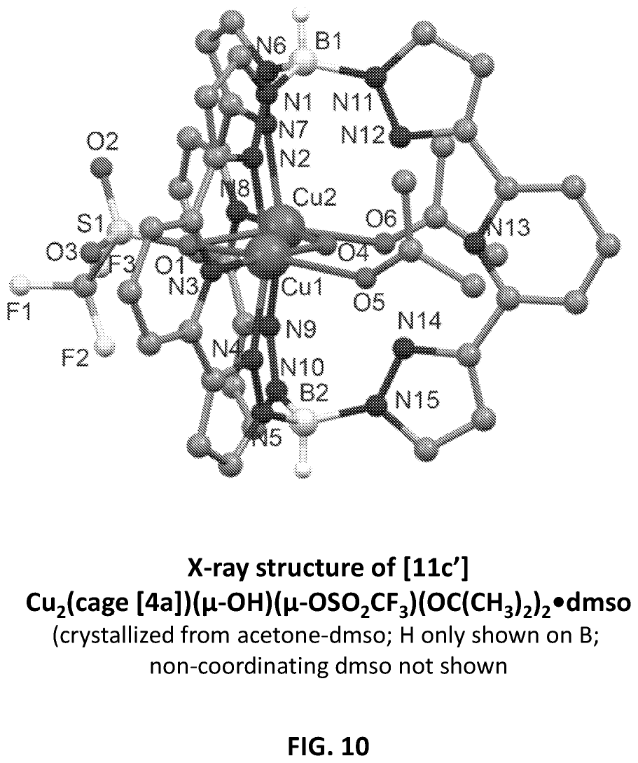

Reaction of Cu(O₃SCF₃)₂ (3 equiv) with [4a] (1 equiv) in acetone produced two products, [11c′] and [11c″]. The less soluble apple green powder [11c′] gave NMR and ESI MS data consistent with the dicopper complex. The more soluble deep blue product [11c″] gave spectroscopic data consistent with the tricopper complex. Yield: 30% (based on formula). Mp. dec.>300° C. ¹H NMR (500 MHz, dmso-d₆: acetone-d₆, 1:1, 298 K): δ 64.87 (4H, br s, 4′- or 5′-pzH, v_{1/2}=55 Hz), 49.19 (4H, br s, 4′- or 5′-pzH, v_{1/2}=30 Hz), 26.96 (4H, br s, 3,5-pyH, v_{1/2}=15 Hz), 12.36 (2H, br s, 4-pyH, v_{1/2}=15 Hz), 8.11 (2H, br d, ³J_{HH}=7.5 Hz, 3,5-pyH), 7.98 (1H, br t, ³J_{HH}=7.5 Hz, 4-pyH), 7.81 (2H, br s, 5′-pzH), 6.53 (2H, br s, 4′-pzH), -0.48 (2H, br s, BH, v_{1/2}=190 Hz); “^{L}B NMR (160.5 MHz, dmso-d₆: acetone-d₆, 1:1, 298 K): δ 12.1 (v_{1/2}=380 Hz). +ESI MS (125V cone voltage, IPA): [Cu₂ cage [4a] (OH)]⁺, observed: 792 (6), 793 (43), 794 (100), 795 (74), 796 (93), 797 (45), 798 (28), 799 (10), 800 (6); calculated: 792 (5), 793 (41), 794 (100), 795 (73), 796 (94), 797 (41), 798 (25), 799 (8), 800 (2). An X-ray crystal structure of [11c′], crystallized from acetone-dmso, is shown in FIG. 10. It contains two copper atoms that are bridged by a hydroxide inside the cage and a triflate outside the cage. Each copper atom also has a bonded acetone, and there is a non-coordinating dmso (not shown). Each Cu²⁺ site is distorted octahedral with mer coordination of one arm (two pz N and one py N).

[Cu₃ Cage [4a] (μ³-O)]²⁺ [CF₃SO₃⁻]₂=Metal Complex [11c″]:

Deep blue powder. Yield: 60% (based on formula). +ESI MS (125V cone voltage, IPA): [Cu₃ cage [4a] (O)(CF₃SO₃)]⁺, observed: 1004 (31), 1005 (76), 1006 (73), 1007 (100), 1008 (60), 1009 (65), 1010 (23), 1011 (16), 1012 (10); calculated: 1004 (28), 1005 (71), 1006 (66), 1007 (100), 1008 (56), 1009 (52), 1010 (22), 1011 (11), 1012 (4).

[Zn₂ Cage [4a] (OH)]⁺ [NO₃⁻]=Metal Complex [11d]:

White powder. Yield: 91% (based on formula). Mp. dec.>300° C. ¹H NMR (300 MHz, 1:1 acetone-d₆:dmso-d₆, 293 K): δ 8.090 (6H, d, ³J_{HH}=2.4 Hz, 5′-pzH), 7.999 (3H, m, 4-pyH), 7.948 (6H, m, 3,5-pyH), 7.027 (6H, d, ³J_{HH}=2.4 Hz, 4′-pzH), 4.940 (2H, br s, BH); +ESI MS (125V cone voltage, MeOH): [Zn₂ cage [4a] (OH)]⁺, observed: 795 (20), 796 (85), 797 (70), 798 (100), 799 (60), 800 (82), 801 (45), 802 (48), 803 (26), 804 (20), 805 (5); calculated: 795 (32), 796 (78), 797 (65), 798 (100), 799 (81), 800 (98), 801 (55), 802 (47), 803 (24), 804 (17), 805 (6).

[Cd₃ Cage [4a]]⁴⁺ [NO₃⁻]₄=Metal Complex [11e]:

White powder. Yield: 65% (based on formula). Mp. dec.>300° C. ¹H NMR (300 MHz, dmso-d₆, 298 K): δ 7.904 (6H, d, ³J_{HH}=2.4 Hz, 5′-pzH), 7.887 (3H, t, ³J_{HH}=7.8 Hz, 4-pyH), 7.236 (6H, d, ³J_{HH}=7.8 Hz, 3,5-pyH), 6.870 (6H, d, ³J_{HH}=2.4 Hz, 4′-pzH), 4.80 (2H, br s, BH); ¹³C{¹H} (75 MHz, dmso-d₆): δ 150.77 (2,6 pyC), 150.16 (3′-pzC), 138.77 (4-pyCH), 138.12 (5′-pzCHl), 119.44 (3,5-pyCH), 103.34 (4′-pzCH); ¹¹B NMR (115.6 MHz, dmso-d₆): δ +0.5 (br s).

[Ag₈ Cage [4a]₂]²⁺ [BF₄⁻]₂·2 H₂O=Metal Complex [11f]:

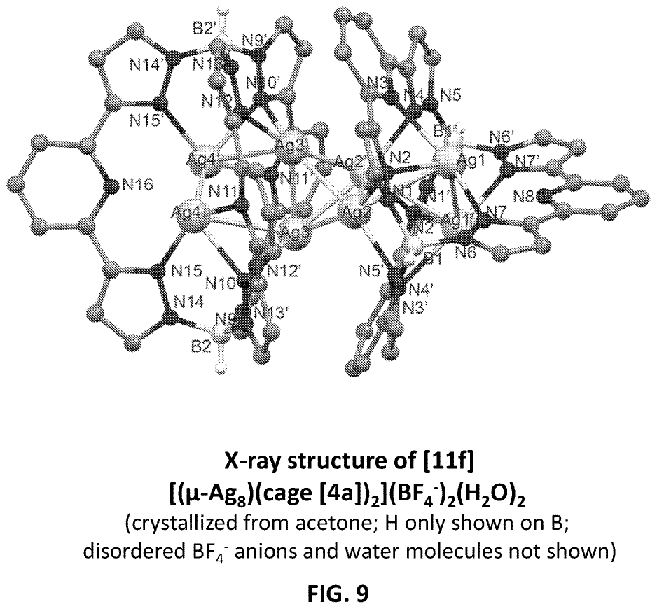

Prepared in a foil-covered flask from a 4:1 ratio of AgBF₄ to K₂(cage [4a]) in acetone. Light sensitive yellow crystals. Yield: 85% (based on formula). ¹H NMR (300 MHz, dmso-d₆, 298 K): δ 8.117 (4H, d, ³J_{HH}=2.1 Hz, 5′-pzH), 7.772 (2H, d, ³J_{HH}=2.1 Hz, 5′-pzH), 7.755 (1H, t, ³J_{HH}=7.8 Hz, 4-pyH), 7.729 (2H, t, ³J_{HH}=7.8 Hz, 4-pyH), 7.543 (2H, d, ³J_{HH}=7.8 Hz, 3,5-pyH), 7.319 (4H, d, ³J_{HH}=7.8 Hz, 3,5-pyH), 6.803 (4H, d, ³J_{HH}=2.1 Hz, 4′-pzH), 6.711 (2H, d, ³J_{HH}=2.1 Hz, 4′-pzH), 4.64 (2H, br s, BH); ¹¹B NMR (115.6 MHz, dmso-d₆): δ −2.10 (br s). An X-ray crystal structure of [11f], crystallized from acetone, is shown in FIG. 9. This interesting structure contains an Ag₈⁶⁺ unit spanning two cages. The Ag₈ unit can be viewed as a central tetrahedron containing Ag-Ag bonds between all members with another two Ag bonded to pairs of Ag in the tetrahedron to form open squares. The BF₄⁻ anions are disordered and there is likely some disordered water, but the overall refinement is acceptable.

[Cu Ring [8a]]=Metal Complex [12a]:

Blue-green powder. Yield: 63% (based on formula). Mp. dec.>300° C. ¹H NMR (500 MHz, 1:1 acetone-d₆:dmso-d₆, 293 K): δ 78.21 (4H, v_{1/2}=115 Hz), 52.96 (4H, v_{1/2}=55 Hz), 39.46 (4H, v_{1/2}=40 Hz), 11.00 (2H, v_{1/2}=18 Hz), −9.32 (2H, BH, v_{1/2}=110 Hz), −16.29 (2H, BH, v_{1/2}=190 Hz); ¹¹B (160.51 MHz, 1:1 acetone-d₆:dmso-d₆, 293 K): δ 59.0); +ESI MS (75V cone voltage, MeOH): [Cu₂ cage [4a] (OCH₃)]⁺, observed: 599 (5), 600 (42), 601 (100), 602 (60), 603 (86), 604 (32), 605 (22), 606 (4); calculated: 599 (5), 600 (43), 601 (100), 602 (64), 603 (90), 604 (32), 605 (22), 606 (5). The observation of two Cu²⁺ ions in the molecular ion and a methoxide group derived from the methanol is likely generated in situ during the ionization process; the observation of two 2H BH₂ resonances in the ¹H NMR is only consistent with a mono Cu ring complex with folded C₂ᵥ symmetry.

[Y Ring [8a]]=Metal Complex [12b]:

White powder. Yield: 65% (based on formula). Mp. dec.>300° C. ¹H NMR (300 MHz, 3:1 acetone-d₆:dmso-d₆, 293 K): δ 8.215 (6H, br s, 5′-pzH), 8.05-8.07 (9H, overlapping 3,5-pyH and 4-pyH), 7.186 (6H, d, ³J_{HH}=2.1 Hz, 4′-pzH), not observed BH (2H, br s, BH).

While particular elements, embodiments and applications of the present technology have been shown and described, it will be understood, that the technology is not limited thereto since modifications can be made by those skilled in the art without departing from the scope of the present disclosure, particularly in light of the foregoing teachings. It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

U.S. Provisional Application 63/322,573, filed Mar. 22, 2022 is incorporated herein by reference, in its entirety.

What is claimed is:

1. A multidentate ligand comprising a first boron atom and a second boron atom, said first and second boron atoms linked by at least two arms, each of said arms comprising a first nitrogen-heterocyclic ring linked by linking moiety to a second nitrogen-heterocyclic ring, wherein said first nitrogen heterocyclic ring of each arm is bonded to said first boron atom via a B—N bond, and said second nitrogen heterocyclic ring of each arm is bonded to said second boron atom via a B—N bond.

2. A multidentate ligand according to claim 1 wherein said linking moiety is aromatic or heteroaromatic link.

3. A multidentate ligand according to claim 2 wherein said multidentate ligand is a clathrochelate ligand in which said first and second boron atoms are linked by three arms, each of said arms comprising said first nitrogen-heterocyclic ring linked by said aromatic or heteroaromatic link to said second nitrogen-heterocyclic ring, wherein said first nitrogen heterocyclic ring of each of said three arms is bonded to said first boron atom via a B—N bond, and said second nitrogen heterocyclic ring of each of said three arms is bonded to said second boron atom via a B—N bond.

4. A multidentate ligand according to claim 2 wherein said multidentate ligand is a macrocyclic ligand in which said first and second boron atoms are linked by two arms, each of said arms comprising said first nitrogen-heterocyclic ring linked by said aromatic or heteroaromatic link to said second nitrogen-heterocyclic ring, wherein said first nitrogen heterocyclic ring of each of said two arms is bonded to said first boron atom via a B—N bond, and said second nitrogen heterocyclic ring of each of said two arms is bonded to said second boron atom via a B—N bond.

5. A multidentate ligand according to claim 2 wherein said aromatic or heteroaromatic link is substantially flat or planar.

6. A multidentate ligand according to claim 2 wherein said aromatic or heteroaromatic link is a pyridine link.

7. A multidentate ligand according to claim 2 wherein said aromatic or heteroaromatic link is a substituted pyridine link.

8. A multidentate ligand according to claim 1 wherein said linking moiety links said first nitrogen-heterocyclic ring to said second nitrogen-heterocyclic ring via a single carbon atom within said linking moiety.

9. A multidentate ligand according to any of claims 1, 2 and 8 wherein each of said arms is the same.

10. A multidentate ligand according to any of claims 1, 2 and 8 wherein one of said at least two arms is different from another of said at least two arms.

11. A multidentate ligand according to any of claims, 2 and 8 wherein said first and second nitrogen-heterocyclic rings are pyrazolyl rings.

12. A clathrochelate ligand having the following formula: 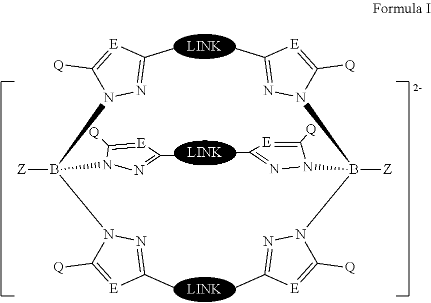
wherein:
E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
Q=H, alkyl, aryl, alkenyl, or alkynyl;
Z═H, alkyl, aryl, alkoxy, N-heterocycle or 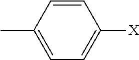
where X═H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK= 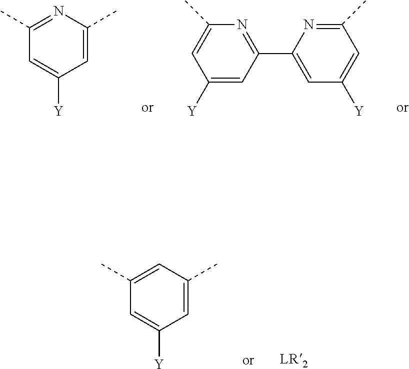
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C, Si or Ge and each R′ is individually selected from H, alkyl or alkoxy.

13. A clathrochelate ligand according to claim 12 wherein:
LINK= 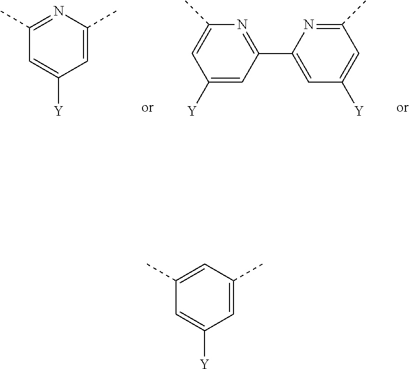
where Y═H, alkyl, aryl, alkenyl, alkynyl, CF₃, F, Cl, Br, I, O(CH₂)ₙR (n=0-4), S(CH₂)ₙR (n=0-4), NH(CH₂)ₙNR₂ (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

14. A clathrochelate ligand according to claim 12 wherein Z═H and L=C.

15. A clathrochelate ligand according to claim 12 wherein Z═H, E=C-J, and L=C.

16. A clathrochelate ligand according to claim 12 wherein Z═H, E=CH, Q=H and and L=C.

17. A clathrochelate ligand according to claim 12 wherein Z═H, E=CH, Q=H, and
LINK= 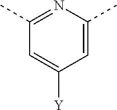
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

18. A clathrochelate ligand according to claim 16 wherein Y═H, alkyl, aryl, alkenyl, CF₃, F, Cl, Br, I, O(CH₂)ₙR (n=0-4), S(CH₂)ₙR (n=0-4), NR(CH₂)ₙNR₂, NH(CH₂)ₙOH (n=0-4), NO₂, SO₃R, CN, C(O)R, C(O)NR₂, C(═NHNH₂)NH₂ (n=0-4), CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, or aryl.

19. A clathrochelate ligand according to claim 18 wherein Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl.

20. A clathrochelate ligand according to claim 16 wherein Y═H.

21. A clathrochelate ligand according to claim 12 wherein Z═H, E=C-J, and Q and/or J≠H, and LINK=2,6 pyridinyl.

22. A clathrochelate ligand according to claim 21 wherein J=H, alkyl, alkenyl, F, Cl, Br, I, SH, NH₂, or C(O)CF₃, and Q=H, alkyl, or alkenyl.

23. A clathrochelate ligand according to claim 22 wherein J=H or Br and Q=Me, or J=Br and Q=H, or J=C(═O)CF₃ and Q=H.

24. A clathrochelate ligand according to claim 12 wherein Z═H, E=CH, Q=H, and LINK= 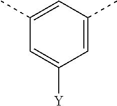
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

25. A clathrochelate ligand according to claim 24 wherein Y═H, alkyl, alkenyl, CF₃, Br, I, NO₂, CN, C(O)Me, C(O)NH₂, (CH₂)OR, OR, (CH₂)SR, SR, NR₂, where each R is individually selected from H, alkyl, alkenyl, or aryl.

26. A clathrochelate ligand according to claim 12 wherein Z═H, E=N, Q=H, and
LINK= 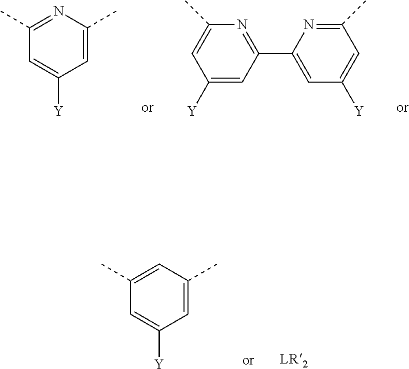
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C and each R′ is individually selected from H, alkyl or alkoxy.

27. A clathrochelate ligand according to claim 26 wherein:
LINK= 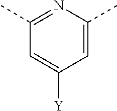
where Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl.

28. A macrocyclic ligand having the following formula: 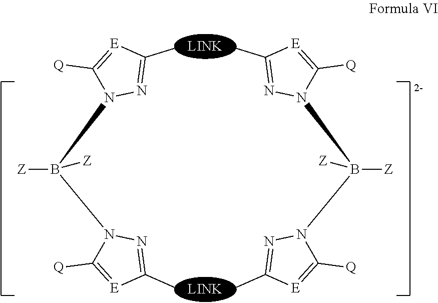
wherein:
E=N or C-J, where none, some or all of J=H, and the remainder are alkyl, aryl, alkenyl, alkynyl, F, Cl, Br, I, SR, NR₂, NO₂, SO₃R, C(O)R, C(O)CF₃, CO₂R, or NRSO₂F, where each R is individually selected from H, alkyl or aryl;
Q=H, alkyl, aryl, alkenyl, or alkynyl;
Z═H, alkyl, aryl, alkoxy, N-heterocycle or 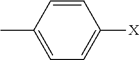
where X═H, alkyl, aryl, alkenyl, or alkynyl, CF₃, F, Cl, Br, I, OR, SR, NR₂, NO₂, SO₃R, CN, C(O)R, C(O)NR₂, CO₂R, CH₂CH₂NR₂, OCH₂CH₂OR, or a heterocyclic ring, where each R is individually selected from H, alkyl or aryl; and
LINK= 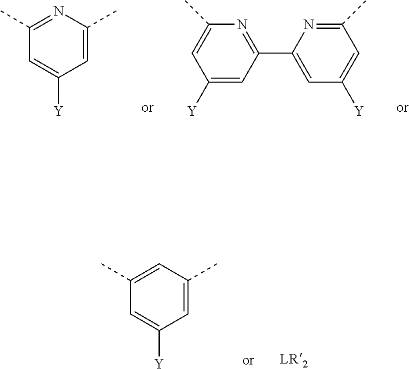
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring,
and where L=C and each R′ is individually selected from H, alkyl or alkoxy.

29. A macrocyclic ligand according to claim 28 wherein at least one of Z is H, and wherein:
LINK= 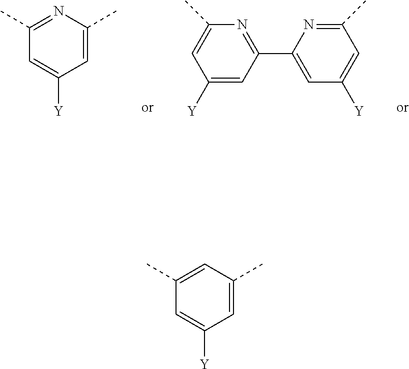
where Y═H, alkyl, aryl, alkenyl, alkynyl, CF₃, F, Cl, Br, I, O(CH₂)ₙR (n=0-4), S(CH₂)ₙR (n=0-4), NH(CH₂)ₙNR₂ (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

30. A macrocyclic ligand according to claim 28 wherein Z═H, and L=C.

31. A macrocyclic ligand according to claim 28 wherein Z═H and E=C-J, and L=C.

32. A macrocyclic ligand according to claim 28 wherein Z═H, E=CH, Q=H, and L=C.

33. A macrocyclic ligand according to claim 28 wherein Z═H, E=CH, Q=H, and
LINK= 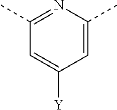
where Y═H, alkyl, aryl, alkenyl, alkynyl, CF₃, F, Cl, Br, I, O(CH₂)ₙR (n=0-4), S(CH₂)ₙR (n=0-4), NR(CH₂)ₙNR₂ (n=0-4), NH(CH₂)ₙOH (n=0-4), NO₂, SO₃R, CN, C(O)R, C(O)NR₂, C(═NHNH₂)NH₂, CO₂Li, CO₂R, (CH₂)ₙOR (n=0-3), (CH₂)ₙSR (n=0-3), (CH₂)ₙNR₂ (n=0-4), O(CH₂)ₙOR (n=1-6), O(CH₂CH₂O)ₙR (n=1-6), S(CH₂)ₙSR (n=1-6), S(CH₂)ₙCHO (n=1-6), S(CH₂)ₙ(1,3-dioxacyclopentane) (n=1-6), or a heterocyclic ring, where each R is individually selected from H, alkyl, alkenyl, aryl, or a heterocyclic ring.

34. A macrocyclic ligand according to claim 33 wherein Y═H, alkyl, alkenyl, CF₃, Br, I, O(CH₂)ₙCH₂ (n=0-4), S(CH₂)R, NH(CH₂)ₙNR₂ (n=0-4), NO₂, CN, C(O)Me, C(O)NH₂, C(═NHNH₂)NH₂, CO₂Li, (CH₂)OR, OR, (CH₂)SR, SR, NR₂, O(CH₂)ₙOH (n=1-6), O(CH₂CH₂O)ₙMe (n=1-6), S(CH₂)ₙSH (n=1-6), S(CH₂)₂CHO, S(CH₂)₂(1,3-dioxacyclopentane), where each R is individually selected from H, alkyl, alkenyl, or aryl.

35. A macrocyclic ligand according to claim 34 wherein Y═H, Cl, Br, I or CN.

36. A metal salt of a ligand of any of claims 1 to 35.

37. A metal complex of a ligand of any of claims 1 to 35.

38. A ligand of any of claims 1 to 35 functionalized to include a connector for binding the ligand to a biomolecule.

39. A metal complex of a ligand of any of claims 1 to 35 functionalized to include a connector for binding the ligand to a biomolecule.

40. A radiopharmaceutical composition comprising a solution of a metal complex of a ligand of any of claims 1 to 35, wherein the metal is a radioactive metal.

41. A radiopharmaceutical composition comprising a solution of a metal complex of a ligand of any of claims 1 to 35, wherein the ligand comprises at least one radioactive atom.

42. A radiopharmaceutical composition comprising a solution of a metal complex of a ligand of any of claims 1 to 35, bound to a biomolecule, wherein the metal is a radioactive metal.

43. A radiopharmaceutical composition comprising a solution of a metal complex of a ligand of any of claims 1 to 35, bound to a biomolecule, wherein the ligand comprises at least one radioactive atom.

44. A radiopharmaceutical composition of claim 42 or 43 where the biomolecule is a monoclonal antibody, and the ligand is bound to the biomolecule via a connector.

45. A kit for preparing an antibody-drug conjugate comprising a radionuclide complex of a ligand of any of claims 1 to 35, the ligand optionally modified to include a connector for binding the ligand to a biomolecule, the kit comprising:
(a) a first solution of a metal salt of the ligand, the ligand optionally modified to include a connector for binding the ligand to a biomolecule, wherein the metal is a displaceable metal; and
(b) a second a solution of a salt of a radionuclide;
wherein upon mixing the first and second, the displaceable metal is displaced by the radionuclide to form the radionuclide complex.

46. A kit according to claim 45, wherein the radionuclide is useful for radiotherapy or diagnostic imaging procedures.

47. A method to treat a patient suffering from a disease condition, the method comprising injecting into the patient an effective amount of a radiopharmaceutical composition of any of claims 40 to 44 in solution.

48. A method to diagnose a condition in a patient, the method comprising injecting into the patient an effective amount of a radiopharmaceutical composition of any of claims 40 to 44 in solution.
